(19) United States
(12) Patent Application Publication
(10) Pub. No.: US 2025/0144358 A1
(43) Pub. Date: May 8, 2025

(54) SYSTEMS AND METHODS FOR VENTILATORY TREATMENT

(71) Applicant: BMC MEDICAL CO., LTD., Beijing (CN)

(72) Inventors: Yunjing CHEN, Tianjin (CN); Jiuzhong LIU, Tianjin (CN); Shusen YUE, Tianjin (CN); Lixin ZHANG, Tianjin (CN); Yuelong LI, Tianjin (CN); Qingsong WANG, Beijing (CN); Ang LI, Tianjin (CN); Zhi ZHUANG, Beijing (CN)

(73) Assignee: BMC MEDICAL CO., LTD., Beijing (CN)

(21) Appl. No.: 18/725,807

(22) PCT Filed: Dec. 23, 2022
(86) PCT No.: PCT/CN2022/141639

§ 371 Date: Jun. 30, 2024

(30) Foreign Application Priority Data
Dec. 31, 2021 (CN) 202123415904.5
Dec. 31, 2021 (CN) 202123422021.7
Dec. 31, 2021 (CN) 202123422519.3
Dec. 31, 2021 (CN) 202123422870.2
Dec. 31, 2021 (CN) 202123439537.2
Dec. 31, 2021 (CN) 202123440277.0
Dec. 31, 2021 (CN) 202123441552.0
Dec. 31, 2021 (CN) 202123442052.9
Dec. 31, 2021 (CN) 202123442258.1
Dec. 31, 2021 (CN) 202123442296.7
Dec. 31, 2021 (CN) 202123447069.3
Dec. 31, 2021 (CN) 202123448504.4
Jun. 24, 2022 (CN) 202221614224.8

(51) Int. Cl. A61M 16/12; A61M 16/00; A61M 16/08; A61M 16/10; A61M 16/20

(52) U.S. Cl. CPC A61M 16/12; A61M 16/0003; A61M 16/0816; A61M 16/109; A61M 16/203; A61M 16/209; A61M 2016/0027; A61M 2016/003; A61M 2202/0007; A61M 2202/0208; A61M 2205/3327; A61M 2205/3344; A61M 2205/42

(57) ABSTRACT

An apparatus for ventilatory treatment is provided. The apparatus may include at least one gas entrance configured to introduce a first gas into the apparatus and introduce a second gas into the apparatus; a gas mixing assembly configured to mix the first gas and the second gas to generate a mixed gas; a gas export configured to discharge a flow of the mixed gas; and a noise reduction assembly configured to reduce noise of the apparatus.

### CROSS-REFERENCE TO RELATED APPLICATIONS

This application claims priority to Chinese Patent Application No. 202123415904.5, filed on Dec. 31, 2021, Chinese Patent Application No. 202123447069.3, filed on Dec. 31, 2021, Chinese Patent Application No. 202123422519.3, filed on Dec. 31, 2021, Chinese Patent Application No. 202123442258.1, filed on Dec. 31, 2021, Chinese Patent Application No. 202123422021.7, filed on Dec. 31, 2021, Chinese Patent Application No. 202123439537.2, filed on Dec. 31, 2021, Chinese Patent Application No. 202123448504.4, filed on Dec. 31, 2021, Chinese Patent Application No. 202123441552.0, filed on Dec. 31, 2021, Chinese Patent Application No. 202123442052.9, filed on Dec. 31, 2021, Chinese Patent Application No. 202123422870.2, filed on Dec. 31, 2021, Chinese Patent Application No. 202123440277.0, filed on Dec. 31, 2021, Chinese Patent Application No. 202123442296.7, filed on Dec. 31, 2021, and Chinese Patent Application No. 202221614224.8, filed on Jun. 24, 2022, the contents of each of which are hereby incorporated by reference in its entirety.

### TECHNICAL FIELD

This disclosure generally relates to treatment, prevention and amelioration of respiratory-related disorders or diseases, and more particularly, relates to systems and methods for ventilatory treatment.

### BACKGROUND

Respiration is significant for the maintenance of the vitality of a subject (e.g., a human body). The respiratory system of the subject can facilitate gas exchange. The nose and/or mouth of the subject form the entrance to the airways of the subject. A range of respiratory disorders or diseases (e.g., apnea, hypopnea, hyperpnea, snore, acute respiratory distress syndrome, pneumonia, pulmonary fibrosis, pulmonary edema, or the like) exist. The respiratory disorders or diseases can threaten the health (and/or life) of the subject. Therefore, it is desirable to develop system(s) and method(s) for ventilatory treatment for the subject.

### SUMMARY

In one aspect of the present disclosure, an apparatus for ventilatory treatment is provided. The apparatus for ventilatory treatment may include at least one gas entrance configured to introduce a first gas into the apparatus and introduce a second gas into the apparatus; a gas mixing assembly configured to mix the first gas and the second gas to generate a mixed gas; a gas export configured to discharge a flow of the mixed gas; and/or a noise reduction assembly configured to reduce noise of the apparatus.

In some embodiments, the apparatus may include a gas importing mechanism configured to import the second gas, from at least one of a first gas source or a second gas source, via a gas outlet port of the gas importing mechanism and a second inlet port of the gas mixing assembly, into the gas mixing assembly.

The gas importing mechanism may include at least one of a first sub-mechanism or a second sub-mechanism, the first sub-mechanism being configured to import the second gas from the first gas source automatically, the second sub-mechanism being configured to import the second gas from the second gas source manually. The gas importing mechanism can be switched between an automatic gas importing manner and a manual gas importing manner, which increases the flexibility of using the first gas source and/or the second gas source, and expands the application scenario of the ventilatory treatment apparatus. The first sub-mechanism and the second sub-mechanism can be integrated into the gas importing mechanism, and can function properly and be switched through controlling the on/off state of a proportional valve, without using additional elements, components, or devices, which saves cost, reduces the weight and volume of the ventilatory treatment apparatus.

In some embodiments, the apparatus may include a gas intake connector assembly configured to introduce the second gas from at least one gas source, via the at least one gas entrance, into the apparatus. The gas intake connector assembly may include a female connector and a male connector operably coupled to the female connector. The gas intake connector assembly may further include a stop mechanism configured to prevent the male connector from being separated with the female connector. In some embodiments, the stop mechanism may include a stop screw and a stop groove. The stop groove may be disposed on the male connector. The stop screw may be capable of entering, via a threaded hole on the female connector, the stop groove. The stop screw may be limited in the stop groove, so that the male connector is not easily separated with the female connector, thereby increasing the stability and safety of the apparatus.

In some embodiments, the ventilatory treatment apparatus may further include or be equipped with one or more gas filter units configured to filter and/or purify the respiratory gas delivered to the subject.

In some embodiments, the ventilatory treatment apparatus may include or be equipped with a radiating tube for dissipating heat. In some embodiments, the radiating tube may be disposed adjacent to a power supply component or pass through the power supply component. Therefore, the gas flowing through the radiating tube may take the heat away from the power supply component, which may decrease the temperature of the power supply component and make the power supply component work in a certain temperature range, thereby improving the work efficiency and stability of the power supply component, improving the service life and stability of the ventilatory treatment apparatus. In addition, the temperature of the gas flowing through the radiating tube may be increased, thereby reducing the thermal power provided by the power supply component.

In some embodiments, the apparatus may include a one-way valve. In a ventilator working mode of the apparatus, when the subject is in the expiratory state, a valve body of the one-way valve may be in the closed state, the gas discharged from the subject may open a reverse valve body of the one-way valve and may pass through the reverse valve body, thereby releasing the pressure in the humidification assembly and the gas passage(s), and alleviating suffocation when the subject exhales. When the apparatus is not in use, the valve body and the reverse valve body may be both in the closed state, thereby preventing the humidified gas in the humidification assembly to flow back into the main body of the apparatus. When the reverse valve body is opened, the left side and the right side of the reverse valve body may have different resonant frequencies, that is, the reverse valve body may have no constant resonant frequency, thereby reducing or eliminating resonance of the reverse valve body, and reducing or avoiding abnormal sound generated by the resonance of the reverse valve body. With the use of a stopping part, the reverse valve body may not cross the valve body to reach the second side, and thus, the reverse valve body may only rotate to the first side to allow the backward respiratory gas to pass through, thereby guaranteeing the airtightness of the one-way valve.

In another aspect of the present disclosure, a noise reduction assembly is provided. The noise reduction assembly may include a noise reduction box configured to accommodate a gas pressurization unit, the noise reduction box including two or more chambers; one or more separation walls configured to define the two or more chambers; and/or one or more connecting pipes configured to direct a gas to flow between the two or more chambers through the one or more separation walls, so that a noise generated by a flow of the gas is reduced or eliminated.

In some embodiments, the two or more chambers may include a third chamber. The noise generated by the gas flowing may be reduced in the two or more chambers with a process of converting sound wave of the noise into heat energy absorbed by the two or more chambers. Further, the gas entering the third chamber may cool a motor of the gas pressurization unit, thus a service life of the gas pressurization unit may be lengthened.

In some embodiments, at least one chamber of the two or more chambers includes one or more guide passages configured to guide the gas to flow in the at least one chamber. The guide passage(s) may be winding, and may be disposed in a first chamber evenly. There may be no bulge or concavity on one or more guide vanes that define the guide passage(s), thus the gas may be divided into a plurality of gases with a substantially uniform speed, and may flow in the one or more guide passages smoothly. Thus, a turbulence may not be generated because of the gases with the substantially uniform speed, and noise generated by the turbulence may be reduced. In addition, corner(s) defined by the guide vane(s) may prevent the noise from spreading out of the noise reduction assembly. Thus, noise generated in the gas inlet port of the first chamber or generated in gas flowing may be reflected a lot of times by inner wall(s) of the corner(s), energy of the noise may thus be consumed, and the noise may be reduced.

In some embodiments, at least one connecting pipe of the one or more connecting pipes may include one or more sub-pipes. At least one sub-pipe of the one or more sub-pipes may be in fluid communication with another sub-pipe of the one or more sub-pipes via a channel. Thus, a resistance for the gas flowing may be reduced, and when the gas passes through the connecting pipe, speeds of different portions of the gas in different portions of the connecting pipe may be substantially uniform. Thus, the gas may flow from a chamber into another chamber stably, and the uniform speed may cause less noise when the gas flowing in the connecting pipe.

In some embodiments, at least one end of at least one connecting pipe may be beveled, thus a range of a wavelength of noise that may be reduced in the connecting pipe may be widened. The effect of noise reduction of a connecting pipe with a beveled end may be better than the effect of noise reduction of a connecting pipe with non-beveled end. In some embodiments, the at least one end of the at least one connecting pipe may be beveled to facilitate the gas to flow.

In some embodiments, the noise reduction assembly may be configured without a sound absorbing cotton. If a sound absorbing cotton is used in a noise reduction assembly, and the sound absorbing cotton is used for a long time, or is sterilized by ozone, particles may be produced from the sound absorbing cotton, and the particles may be spread into the gas. Since no sound absorbing cotton is provided in the noise reduction assembly, particles in a pressurized gas supplied by the gas pressurization unit may be reduced, thus the quality of the pressurized gas may be improved.

In another aspect of the present disclosure, a humidification assembly for humidifying a gas is provided. The humidification assembly may include a liquid chamber configured to accommodate a liquid, the liquid being configured to humidify the gas to generate a humidified gas, wherein the liquid chamber includes a tank including a tank body and a tank bottom, and a gas transmission interface configured to facilitate a gas communication between the tank body with an apparatus for ventilatory treatment and/or a respiration tube.

In some embodiments, the tank body may include a separation board configured to increase a length of a flow path of the gas inside the tank body, so that the gas is fully humidified and/or warmed.

In some embodiments, the gas transmission interface may include a gas intake tube and a gas exit tube.

In some embodiments, the separation board may include a plurality of blocking portions. A plurality of gas flow channels may be formed between any two adjacent blocking portions. In some embodiments, a portion of the gas in the tank may pass through the plurality of gas flow channels and flow to the gas exit tube. Another portion of the gas in the tank may be blocked by the plurality of blocking portions and flow downward, so as to improve a humidification effect of the gas with the liquid in the tank and reduce a resistance of the gas flowing from the gas intake tube to the gas exit tube.

In some embodiments, at least one of the gas intake tube or the gas exit tube may include one or more holes. The one or more holes may be configured to facilitate at least a portion of the gas flowing from the gas intake tube to flow to an upper wall (or the top wall) of the liquid chamber to drive at least a portion of the gas in an upper portion of the liquid chamber to flow to a bottom portion of the liquid chamber, so that the at least a portion of the gas may be completely mixed with the gas in the tank body, thereby improving the a humidification effect of the gas.

In some embodiments, the ventilatory treatment apparatus may further include a liquid flow detection device (e.g., a photoelectric sensor) and/or a liquid filling tube. When the liquid flow detection device detects that no liquid flows in the liquid filling tube, the external liquid source may be replaced in time, thereby preventing the tank from heating up without liquid, so as to improve the safety of the ventilatory treatment apparatus.

In some embodiments, the photoelectric sensor may include a slot. The liquid filling tube may be fixed by the slot, avoiding a relative movement of the liquid filling tube, so as to improve an accuracy of the detection result.

The photoelectric sensor may include an infrared photoelectric sensor. The infrared photoelectric sensor may detect the change of the liquid flow in the liquid filling tube through a non-contact detection manner, thereby avoiding liquid contamination during the detection and improving the security of the apparatus.

In some embodiments, the tank may be rotated relative to the ventilatory treatment apparatus, so as to increase a distance between the tank and the ventilatory treatment apparatus, thereby disposing a gas filter unit between the gas intake tube and the gas outlet port of the main body of the ventilatory treatment apparatus.

In another aspect of the present disclosure, a heater assembly for heating a liquid is provided. The heater assembly may include a heat transfer plate configured to heat the liquid and generate vapor to humidify a gas above the liquid; a heater configured to heat the heat transfer plate; and/or an overheat protection mechanism configured to protect the heater from overheating.

In some embodiments, the heater assembly may further include a fastening piece configured to fasten the heater and the overheat protection mechanism onto the heat transfer plate. The heater and the overheat protection mechanism may be sandwiched between the fastening piece and the heat transfer plate, so that the overheat protection mechanism may be tightly attached to the heat transfer plate through the fastening piece, thereby preventing impurities from entering between the overheat protection mechanism and the heat transfer plate, ensuring an accurate detected temperature determined by the overheat protection mechanism, and improving the overheat protection performance of the overheat protection mechanism.

In some embodiments, the overheat protection mechanism may include a first temperature sensor and a fuse, the first temperature sensor being configured to detect a temperature of the heat transfer plate, the fuse being configured to provide overcurrent protection of the heater through fusing.

In some embodiments, the overheat protection mechanism may further include a second temperature sensor configured to detect a temperature of the heat transfer plate.

In some embodiments, when the first temperature sensor detects the temperature of the heat transfer plate exceeds a first threshold, the overheat protection mechanism protects the heater from overheating through software control (e.g., performing a first level overheat protection by cutting off a connection between the first temperature sensor and a control circuit to stop the heating of the heater, the control circuit being configured to control a working status of the heater).

In some embodiments, when the second temperature sensor detects the temperature of the heat transfer plate exceeds a second threshold, the overheat protection mechanism protects the heater from overheating through hardware control. The hardware control may be performed by using a voltage comparator to cut off a heating control logic of the control circuit.

If the first temperature sensor detects the temperature of the heat transfer plate exceeds the first threshold, the overheat protection mechanism may perform the first level overheat protection by cutting off a connection between the first temperature sensor and the control circuit to stop the heating of the heater. If the first level overheat protection fails and the second temperature sensor detects the temperature of the heat transfer plate exceeds the second threshold, the overheat protection mechanism may perform the second level overheat protection by cutting off a connection between the second temperature sensor and the control circuit to stop the heating of the heater. When the first level overheat protection and the second level overheat protection fail, and the temperature of the heat transfer plate exceeds the third threshold, the overheat protection mechanism may perform the third level overheat protection by cutting off a connection between the fuse and the control circuit to provide the overcurrent protection of the heater through fusing the fuse. With the first level overheat protection, the second level overheat protection, and the third level overheat protection, the protection of the heater may be improved, and the safety of the apparatus can be improved.

In another aspect of the present disclosure, a power supply assembly is provided. The power supply assembly may include an isolated power supply component configured to supply power to a system load of an apparatus for ventilatory treatment; a non-isolated power supply component configured to supply power to a heating load of the apparatus; wherein the isolated power supply component and the non-isolated power supply component are integrated in a single circuit board and share at least a portion of an electromagnetic compatibility (EMC) circuit and/or at least a portion of a heat dissipation mechanism.

With the isolated power supply component and the non-isolated power supply component, requirements of power supply of different components or devices of the ventilatory treatment apparatus may be satisfied, and the apparatus may be suitable for more application scenarios with different input voltages, thus the work efficiency of the apparatus may be improved. The isolated power supply component and the non-isolated power supply component may be integrated in a single circuit board and share at least a portion of the EMC circuit and at least a portion of the heat dissipation mechanism, a space utilization of a circuit board of the power supply assembly may be improved, a size of the circuit board of the power supply assembly may be reduced, and thus the installation of the power supply assembly may be more convenient, and a production cost of the circuit board may be decreased. The EMC circuit may be provided near a high voltage circuit (e.g., a circuit of the non-isolated power supply component) on the circuit board of the power supply assembly, thus a rule scope of a circuit of the non-isolated power supply component may be narrowed, and the space utilization may further be improved.

In some embodiments, the isolated power supply component may include a rectifier circuit configured to rectify AC to DC, an input filter, a transformer configured to facilitate voltage transformation, and an output filter. In some embodiments, the transformer may be a coupling of a plurality of inductance coils, thus the input circuit of the isolated power supply component may not be connected with an output circuit of the isolated power supply component directly, but the input circuit may be in electrical communication with the output circuit with the plurality of inductance coils. In this way, the high AC voltage inputted to the input circuit may be rectified to the low DC voltage in the output circuit, and different circuits in the isolated power supply component may not be affected by each other. An operator (e.g., a user of the ventilatory treatment apparatus) may not need to operate the isolated power supply component in a high voltage (e.g., mains electricity with 220V), and thus, the operation safety may be improved.

The transmission efficiency of electric signals and electric energy may be improved because of a direct connection of the input circuit and the output circuit, and the difficulty of designing the circuit may be reduced, and the cost of producing the non-isolated power supply component may also be decreased.

In some embodiments, the safety capacitor may include an X-capacitor disposed between a zero line and a fire line, and is in parallel connection with a magnet ring of the EMC circuit. The X-capacitor may be configured to avoid electromagnetic interference (EMI) generated by other components in the EMC circuit, thus to improve a performance of anti-interference capability of the EMC circuit. The service life of the components of the EMC circuit may be improved, and the operation safety may also be improved.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary system for ventilatory treatment according to some embodiments of the present disclosure;

FIGS. 2A-2F illustrate an exemplary ventilatory treatment apparatus according to some embodiments of the present disclosure;

FIGS. 2G-2Q illustrate exemplary filter shells of an exemplary gas filter unit according to some embodiments of the present disclosure;

FIGS. 3A-3G illustrate an exemplary gas filter unit according to some embodiments of the present disclosure;

FIGS. 4A-4B illustrate an exemplary displaying screen according to some embodiments of the present disclosure;

FIGS. 5A-5B illustrate an exemplary electrical interface of the ventilatory treatment apparatus according to some embodiments of the present disclosure;

FIGS. 6A-6C illustrate an exemplary ventilatory treatment apparatus with a radiating tube according to some embodiments of the present disclosure;

FIGS. 7A-7E illustrate an exemplary gas importing mechanism according to some embodiments of the present disclosure;

FIGS. 8A-8C illustrate an exemplary gas intake connector assembly according to some embodiments of the present disclosure;

FIGS. 9A-9R illustrate an exemplary one-way valve according to some embodiments of the present disclosure;

FIGS. 10 and 11 illustrate an exemplary ventilatory treatment apparatus according to some embodiments of the present disclosure;

FIGS. 12-14 illustrate overall views of an exemplary ventilatory treatment apparatus according to some embodiments of the present disclosure;

FIG. 15 illustrates an exemplary humidification assembly according to some embodiments of the present disclosure;

FIG. 16 illustrates an exemplary gas filter unit according to some embodiments of the present disclosure;

FIG. 17 illustrates an exemplary filter box of a gas filter unit according to some embodiments of the present disclosure;

FIGS. 18-21 illustrate an exemplary ventilatory treatment apparatus according to some embodiments of the present disclosure;

FIG. 22 illustrates an exemplary humidification assembly according to some embodiments of the present disclosure;

FIG. 23 illustrates an exemplary gas filter unit according to some embodiments of the present disclosure;

FIG. 24 illustrates an exemplary gas mixing system for ventilatory treatment according to some embodiments of the present disclosure;

FIGS. 25A-25B illustrate an exemplary noise reduction assembly according to some embodiments of the present disclosure;

FIGS. 26A-26B illustrate an exemplary noise reduction assembly according to some embodiments of the present disclosure;

FIGS. 27A-27B illustrate another exemplary noise reduction assembly according to some embodiments of the present disclosure;

FIG. 28 illustrates exemplary guide rib(s) according to some embodiments of the present disclosure;

FIG. 29 illustrates exemplary guide passages in a chamber of a noise reduction box according to some embodiments of the present disclosure;

FIG. 30 is an exploded view of an exemplary noise reduction assembly according to some embodiments of the present disclosure;

FIGS. 31A-31B illustrate an exemplary connecting pipe according to some embodiments of the present disclosure;

FIGS. 32A-32C illustrate an exemplary connecting pipe and an exemplary position-limit mechanism according to some embodiments of the present disclosure;

FIGS. 33A-33B illustrate exemplary distribution of two or more chambers and one or more connecting pipes in a noise reduction box according to some embodiments of the present disclosure;

FIGS. 34A-34B illustrate another exemplary connecting pipe viewing from two different sides of a separation wall according to some embodiments of the present disclosure;

FIG. 35 illustrates another exemplary connecting pipe according to some embodiments of the present disclosure;

FIGS. 36A-36B illustrate another exemplary noise reduction assembly according to some embodiments of the present disclosure;

FIG. 36C shows another exemplary flow direction of a gas in a noise reduction box of the noise reduction assembly;

FIG. 37A illustrates an exemplary humidification assembly according to some embodiments of the present disclosure;

FIG. 37B illustrates an exemplary ventilatory treatment apparatus without the humidification assembly shown in FIG. 37A according to some embodiments of the present disclosure;

FIG. 38 illustrates an exemplary humidification assembly with a separation board according to some embodiments of the present disclosure;

FIG. 39 illustrates a gas flow of an exemplary humidification assembly with a separation board from a top view according to some embodiments of the present disclosure;

FIG. 40A illustrates a gas flow of an exemplary humidification assembly with a separation board from a side view when the separation board is lower than a liquid plane according to some embodiments of the present disclosure;

FIG. 40B illustrates a gas flow of an exemplary humidification assembly with a separation board from a side view when the separation board is higher than a liquid plane according to some embodiments of the present disclosure;

FIG. 41 illustrates an exemplary separation board including a plurality of blocking portions according to some embodiments of the present disclosure;

FIGS. 42A and 42B illustrate an exemplary tank without a separation board according to some embodiments of the present disclosure;

FIG. 43A illustrates an exemplary gas transmission interface according to some embodiments of the present disclosure;

FIG. 43B illustrates an exemplary gas intake tube according to some embodiments of the present disclosure;

FIGS. 44A-45D illustrate exemplary opening directions of a notch of a gas intake tube and a notch of a gas exit tube according to some embodiments of the present disclosure;

FIGS. 46A and 46B illustrate an exemplary first connecting tube according to some embodiments of the present disclosure;

FIGS. 47A and 47B illustrate an exemplary electrical interface of the ventilatory treatment apparatus according to some embodiments of the present disclosure;

FIGS. 48A and 48B illustrate an exemplary connecting adapter according to some embodiments of the present disclosure;

FIG. 49 illustrates another exemplary connecting adapter according to some embodiments of the present disclosure;

FIGS. 50A-50B illustrate an exemplary ventilatory treatment apparatus according to some embodiments of the present disclosure;

FIG. 51 illustrates an exemplary liquid flow detection device and an exemplary liquid filling tube of the ventilatory treatment apparatus according to some embodiments of the present disclosure;

FIG. 52 illustrates an exemplary liquid flow detection device with an alarming device according to some embodiments of the present disclosure;

FIG. 53 illustrates an enlarged view of the liquid flow detection device shown in FIG. 52 according to some embodiments of the present disclosure;

FIGS. 54A-54B illustrate an exemplary temperature measurement sensor according to some embodiments of the present disclosure;

FIGS. 55A-55C illustrate an exemplary heater assembly according to some embodiments of the present disclosure;

FIGS. 56A-56B illustrate rear views of the heater assembly 5500 shown in FIGS. 55A-55C according to some embodiments of the present disclosure;

FIG. 57 illustrates a circuit connection of an overheat protection mechanism according to some embodiments of the present disclosure;

FIGS. 58A-58B illustrate an exemplary heater assembly mounted on a base of a ventilatory treatment apparatus according to some embodiments of the present disclosure;

FIG. 59 illustrates an exploded view of the heater assembly mounted on the base shown in FIGS. 58A-58B according to some embodiments of the present disclosure;

FIG. 60 illustrates an exemplary power supply assembly according to some embodiments of the present disclosure;

FIG. 61 illustrates an exemplary power supply assembly 61200 according to some embodiments of the present disclosure; and

FIG. 62 illustrates an exemplary circuit of a power supply assembly according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is to describe particular exemplary embodiments only and is not intended to be limiting. As used herein, the singular forms “a,” “an,” and “the” may be intended to include the plural forms as well, unless the context expressly indicates otherwise. It will be further understood that the terms “comprise,” “comprises,” and/or “comprising,” “include,” “includes,” and/or “including,” when used in the present disclosure, specify the presence of stated features, integers, steps, operation, elements, and/or components, but do not preclude the presence or addition of one or more other features, integers, steps, operation, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of the present disclosure. It is to be expressly understood, however, that the drawings are for illustration and description only, and are not intended to limit the scope of the present disclosure. It is understood that the drawings are not to scale.

It will be understood that the term “system,” “engine,” “unit,” and/or “module” used herein are one method to distinguish different components, elements, parts, sections, or assemblies of different levels in ascending order. However, the terms may be displaced by other expressions if they achieve the same purpose.

It will be understood that when a unit, engine, or module is referred to as being “on,” “connected to,” or “coupled to,” another unit, engine, or module, it may be directly on, connected or coupled to, or communicate with the other unit, engine, or module, or an intervening unit, engine, or module may be present, unless the context clearly indicates otherwise. As used herein, the term “and/or” includes any and all combinations of one or more of the associated listed items.

The flowcharts used in the present disclosure illustrate operation that systems implement according to some embodiments of the present disclosure. It is to be expressly understood, the operation of the flowcharts may be implemented not in order. Conversely, the operation may be implemented in inverted order, or simultaneously. Moreover, one or more other operation may be added to the flowcharts. One or more operations may be omitted from the flowcharts.

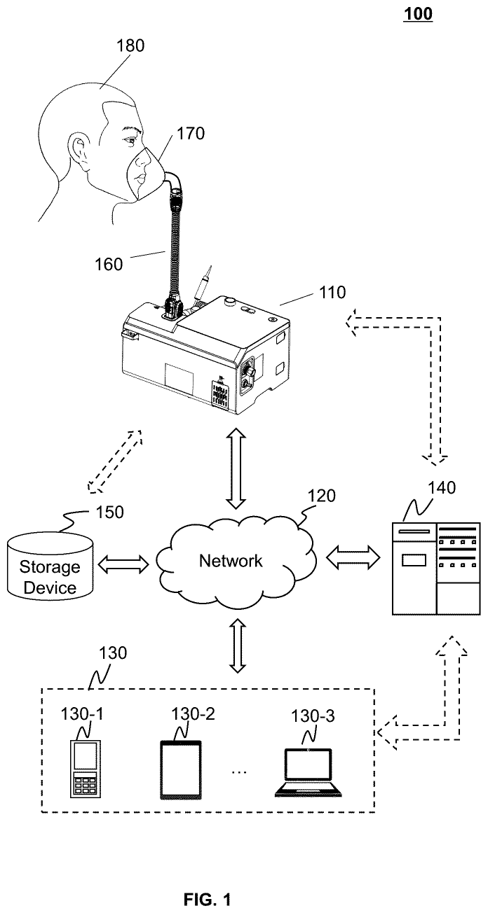

FIG. 1 is a schematic diagram illustrating an exemplary system for ventilatory treatment according to some embodiments of the present disclosure. The system 100 may be configured to provide a respiratory gas to a subject. In some embodiments, the respiratory gas may include natural air (or atmospheric air), purified air, oxygen, atmospheric air enriched with oxygen, a therapeutic drug, pressurized air (enriched with oxygen), humidified air (enriched with oxygen), or the like, or a combination thereof. As illustrated, the system 100 may include a ventilatory treatment apparatus 110, a respiration tube 160, and a subject interface 170. In some embodiments, the ventilatory treatment apparatus 110 may be a non-invasive ventilator, an oxygen therapy device, or any other breathing assistance apparatus. In some embodiments, the system 100 may further include a network 120, a terminal 130, a processing device 140, and a storage device 150. It should be noted that one or more of the network 120, the terminal 130, the processing device 140, and the storage device 150 may be omitted. The components in the system 100 may be connected in one or more of various ways. Merely by way of example, as illustrated in FIG. 1, the ventilatory treatment apparatus 110 may be connected to the processing device 140 through the network 120. As another example, the ventilatory treatment apparatus 110 may be connected to the processing device 140 directly as indicated by the bi-directional arrow in dotted lines linking the ventilatory treatment apparatus 110 and the processing device 140. As a further example, the storage device 150 may be connected to the processing device 140 directly or through the network 120. As still a further example, the terminal 130 may be connected to the processing device 140 directly (as indicated by the bi-directional arrow in dotted lines linking the terminal 130 and the processing device 140) or through the network 120.

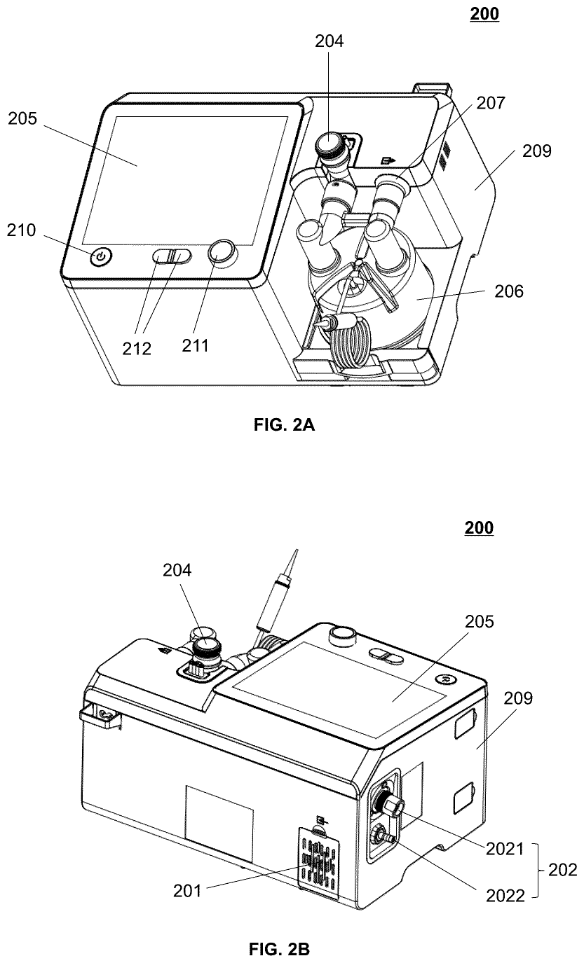

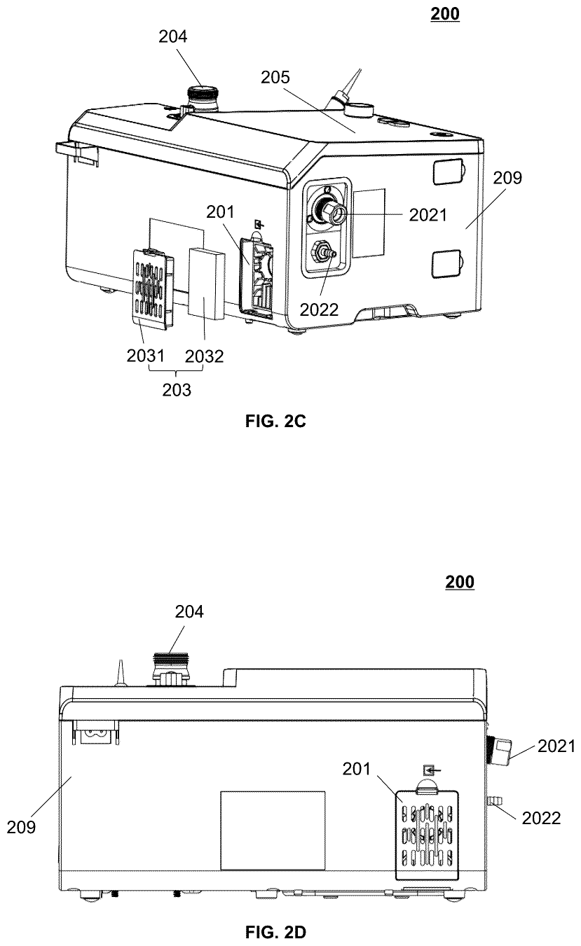

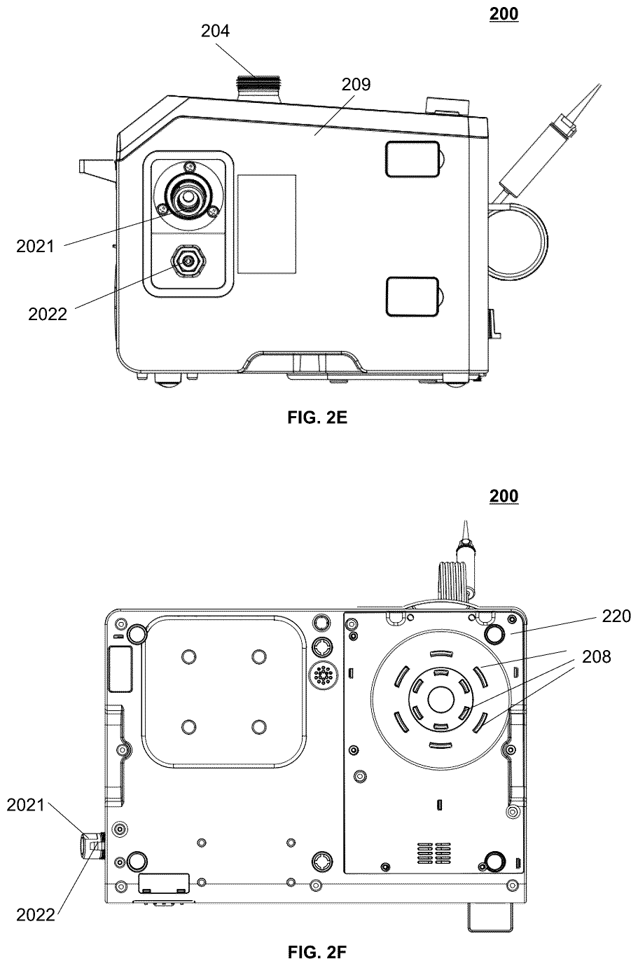

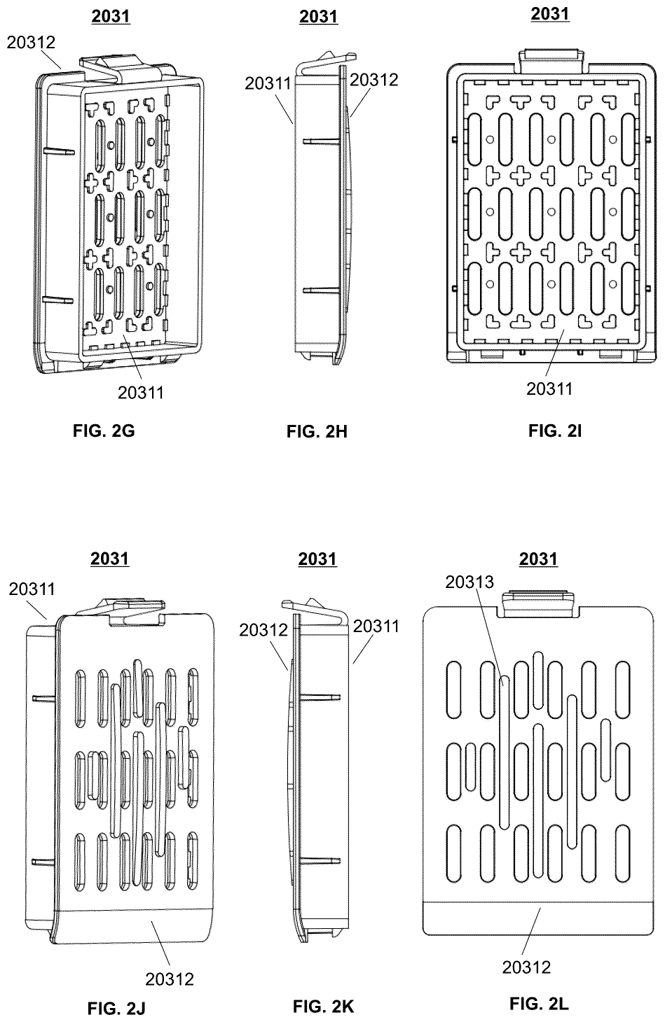

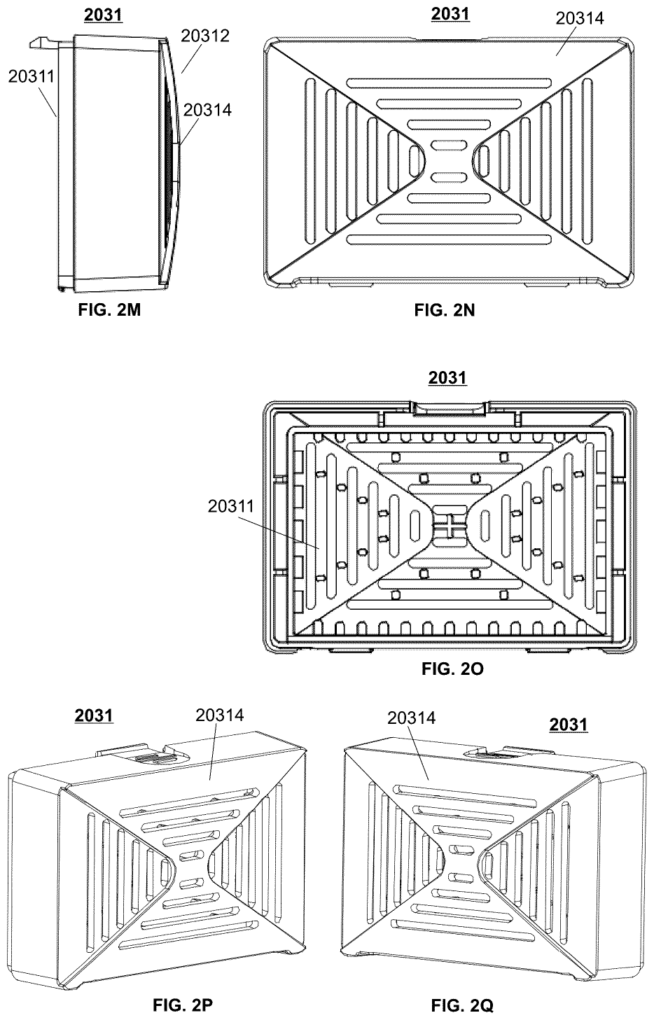

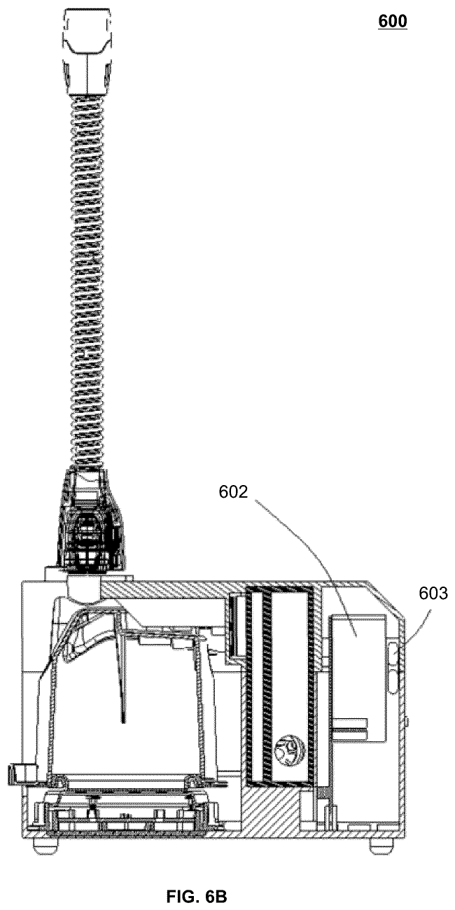

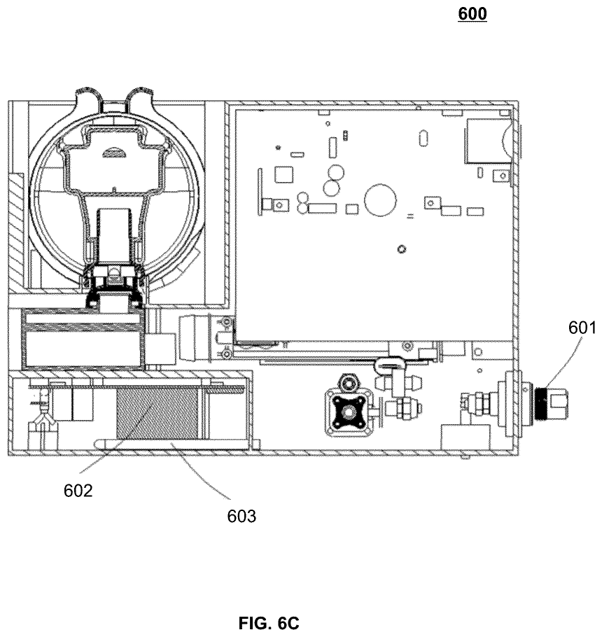

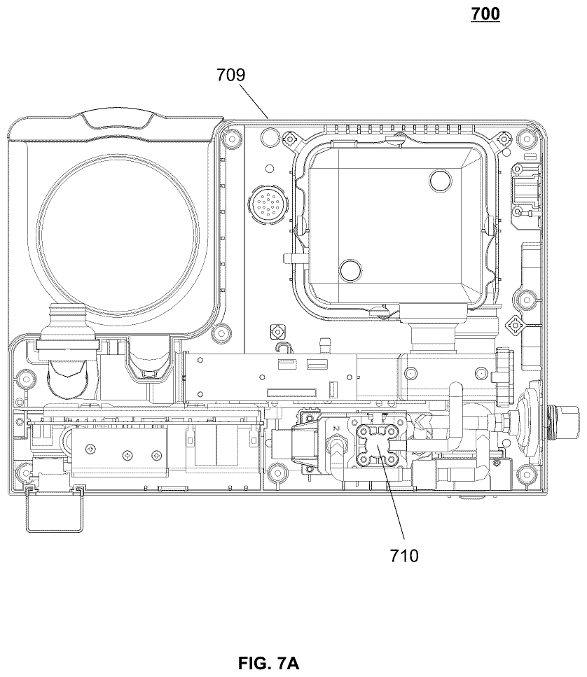

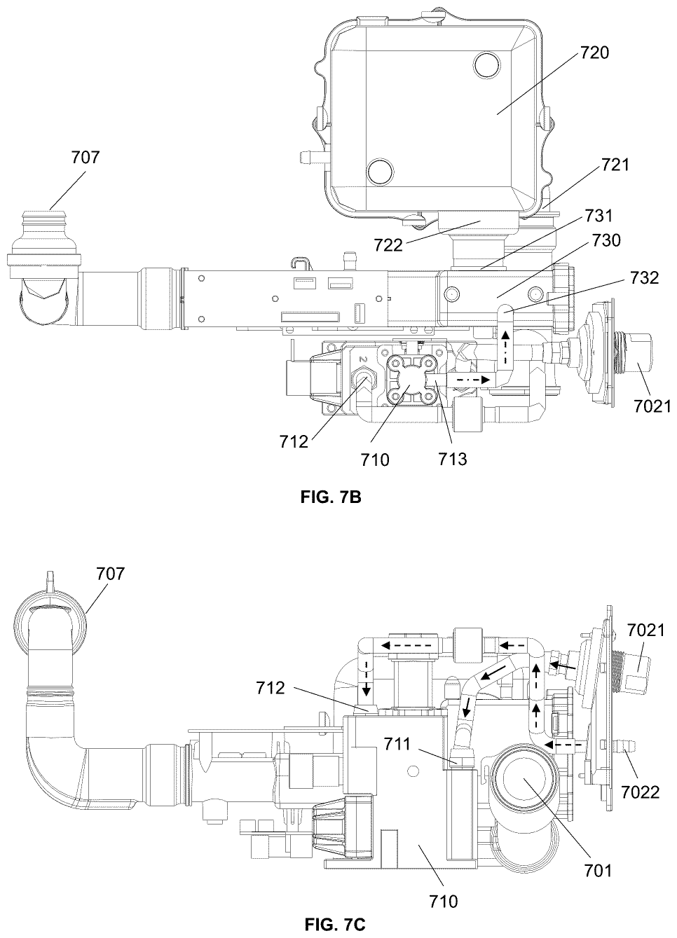

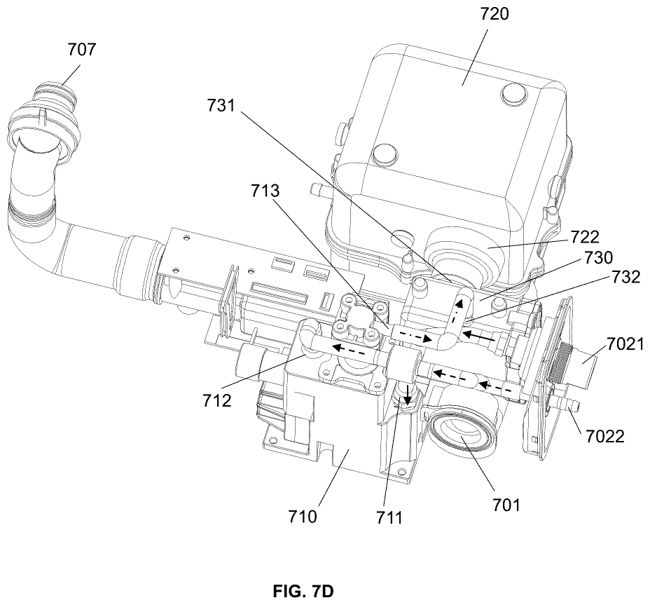

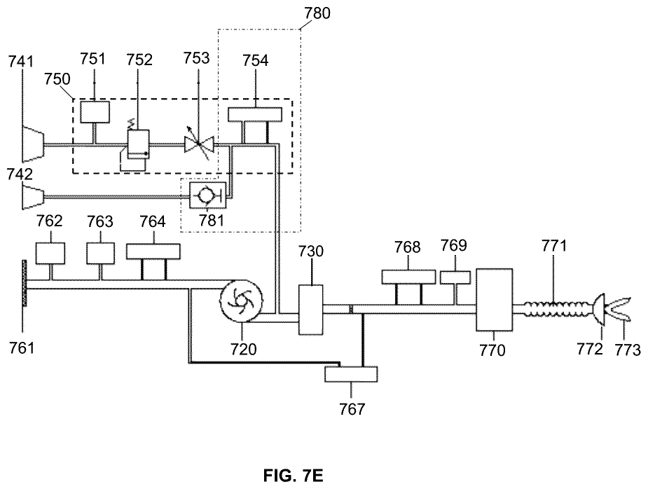

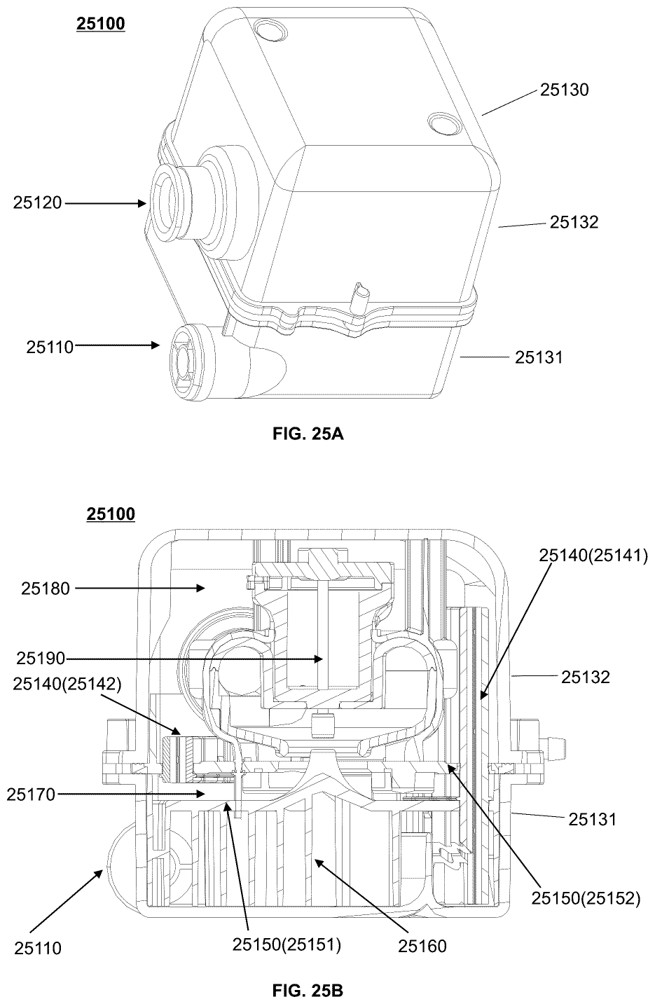

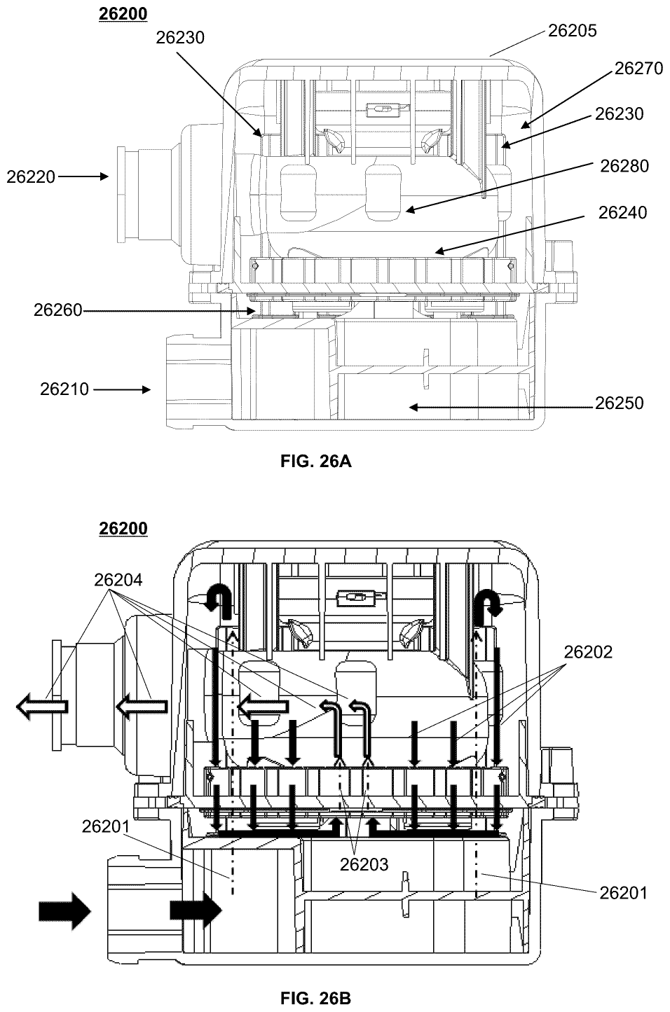

The ventilatory treatment apparatus 110 may be configured to treat, prevent, and/or ameliorate respiratory-related disorders or diseases of a subject 180. In some embodiments, the ventilatory treatment apparatus 110 may deliver a pressurized respiratory gas to a subject 180 (e.g., the nose and/or the mouth of the subject 180). In some embodiments, the ventilatory treatment apparatus 110 may include at least one gas entrance (see FIGS. 2B-2F) and a gas export (see the gas export 204 in FIGS. 2B-2E). The at least one gas entrance may be configured to introduce a first gas into the apparatus and/or introduce a second gas into the apparatus. The gas export may be configured to discharge a flow of a mixed gas (e.g., a mixed gas of the first gas and the second gas, or a mixed gas of the first gas, the second gas, and/or a third gas, etc.) from the apparatus to, for example, the respiration tube 160. An exemplary gas mixing system may be found in FIG. 24. In some embodiments, the ventilatory treatment apparatus 110 may include a gas mixing assembly (see FIGS. 7B-7E) configured to mix the first gas and the second gas to generate the mixed gas. In some embodiments, the gas export may be connected to the respiration tube 160. In some embodiments, the respiration tube 160 may be connected to the subject interface 170. Therefore, the respiratory gas (e.g., the mixed gas, a humidified gas) generated by the ventilatory treatment apparatus 110 may be discharged to the subject 180 via the respiration tube 160 and the subject interface 170. In some embodiments, the ventilatory treatment apparatus 110 may include one or more gas passages (not shown in FIG. 1) configured to guide the respiratory gas to flow in the ventilatory treatment apparatus 110. In some embodiments, the ventilatory treatment apparatus 110 may include a humidification assembly (see FIGS. 15, 22, and 37A-40B) configured to humidify the mixed gas discharged from the gas mixing assembly. In some embodiments, the ventilatory treatment apparatus 110 may include a gas pressurization unit (see FIGS. 25A-27B, 30, and 36A-36C). The gas pressurization unit may be configured to generate a pressurized first gas by pressurizing the first gas before the gas mixing assembly mixes the first gas and the second gas. Alternatively, the gas pressurization unit may be configured to generate a pressurized mixed gas by pressurizing the mixed gas after the gas mixing assembly mixes the first gas and the second gas. In some embodiments, the ventilatory treatment apparatus 110 may include a noise reduction assembly (see FIGS. 25A-27B, 30, and 36A-36C) configured to reduce noise(s) of the apparatus. The noise(s) may be generated by the gas pressurization unit when pressurizing the first gas or the mixed gas. More descriptions of the ventilatory treatment apparatus 110 may be found elsewhere in the present disclosure (e.g., FIGS. 2A-2F and 6A-6C and the descriptions thereof).

In some embodiments, the ventilatory treatment apparatus 110 may further include one or more controllers. The controllers may connect to one or more components of the ventilatory treatment apparatus 110 directly or via a network (e.g., a wired network, a wireless network). The controllers may control the operation(s) of one or more components of the ventilatory treatment apparatus 110. In some embodiments, the controller(s) may be configured to initiate the ventilatory treatment apparatus 110 upon a boot operation. For example, the controller(s) may initiate a random-access memory of the ventilatory treatment apparatus 110, read one or more parameters from one or more storage device 150 (e.g., a non-volatile memory) of the ventilatory treatment apparatus 110, and/or initiate a detection module configured to detect one or more parameters relating to the system 100. In some embodiments, the parameter(s) may include at least one parameter used to control the pressure of the respiratory gas, at least one parameter used to control a humidity of the respiratory gas, at least one parameter used to control a temperature of the respiratory gas, at least one parameter used to control a concentration ratio of a target gas (e.g., oxygen) in the respiratory gas, at least one parameter used to control a flux of the target gas or the respiratory gas, etc. In some embodiments, the controller(s) may be configured to initiate a program that constantly reads information from the detection module, and control parameter(s) of the respiratory gas using at least the information read from the detection module and one or more of the parameters.

In some embodiments, the ventilatory treatment apparatus 110 may further include or be equipped with one or more sensors configured to detect parameters relating to the respiratory gas, the expired gas of the subject 180, and/or the operation status of the ventilatory treatment apparatus 110. The parameters relating to the respiratory gas may include, for example, the flux of the respiratory gas or the target gas, a concentration ratio of the target gas in the respiratory gas, a flow rate of the respiratory gas, a temperature of the respiratory gas, a humidity of the respiratory gas, or the like, or a combination thereof. The parameters relating to the expired gas of the subject 180 may include a respiratory rate of the subject 180, a tidal volume of the subject 180, a pressure of the expired gas of the subject 180, an air leakage of the expired gas of the subject 180, an autonomous respiration ratio of the subject 180, or the like, or a combination thereof. The parameters relating to the operation status of the ventilatory treatment apparatus 110 may include a running time of the ventilatory treatment apparatus 110, a time of delay for pressurizing the respiratory gas, an air leakage of the pressurized respiratory gas, an input voltage of the gas pressurization unit, or the like, or a combination thereof.

In some embodiments, the ventilatory treatment apparatus 110 may further include or be equipped with one or more gas filter units configured to filter and/or purify the respiratory gas delivered to the subject 180. In some embodiments, the gas filter unit(s) (e.g., a coarse filter, a fine filter, or the like) may filter one or more particles in the respiratory gas. In some embodiments, the gas filter unit(s) may filter bacteria in the respiratory gas. In some embodiments, the gas filter unit(s) may filter pungent gas in the respiratory gas.

In some embodiments, the subject 180 may be a patient. In some embodiments, the patient may have one or more respiratory-related disorders or diseases. In some embodiments, the respiratory-related disorders or diseases may be characterized by apneas, hypopneas, or hyperpneas, or the like. Exemplary respiratory-related disorders or diseases may include, for example, obstructive sleep apnea (OSA), Cheyne-stokes respiration (CSR), obesity hyperventilation syndrome (OHS), chronic obstructive pulmonary disease (COPD), neuromuscular disease (NMD), chest wall disorders, acute respiratory distress syndrome (ARDS), or the like. The obstructive sleep apnea (OSA) is a form of sleep disordered breathing, and may cause affected patient to stop breathing for one or more periods (e.g., 30 to 120 seconds duration, or 200 to 300 times per night). The Cheyne-stokes respiration (CSR) is another form of sleep disordered breathing, and may be harmful because of repetitive hypoxia. The obesity hyperventilation syndrome (OHS) is defined as the combination of severe obesity and awake chronic hypercapnia, and may cause dyspnea, morning headache, excessive daytime sleepiness, or the like. The chronic obstructive pulmonary disease (COPD) may include increased resistance to air movement, extended expiratory phase of respiration, or loss of the normal elasticity of the lung, or the like. The chronic obstructive pulmonary disease (COPD) may cause dyspnea on exertion, chronic cough, sputum production, or the like. The neuromuscular disease (NMD) may include diseases and ailments that impair the functioning of the muscles either directly via intrinsic muscle pathology, or indirectly via nerve pathology. The neuromuscular disease (NMD) may cause increasing generalized weakness, dysphagia, dyspnea on exertion and at rest, fatigue, sleepiness, morning headache, difficulties with concentration and mood changes, or the like. The chest wall disorders are a group of thoracic deformities that result in inefficient coupling between respiratory muscles and the thoracic cage. The chest wall disorders may cause dyspnea on exertion, peripheral edema, orthopnea, repeated chest infections, morning headaches, fatigue, poor sleep quality, loss of appetite, or the like. The acute respiratory distress syndrome (ARDS) may be life-threatening condition characterized by poor oxygenation and non-compliant or “stiff” lungs.

In some embodiments, the subject interface 170 may be configured to interface the ventilatory treatment apparatus 110 to the subject 180, for example, by providing a flow of respiratory gas (e.g., air, a humidified air enriched with oxygen). In some embodiments, the subject interface 170 may include a gas passage to guide the respiratory gas. The subject interface 170 may include a mask, a tube, or the like. For example, the subject interface 170 may be a nasal mask, a full-face mask, a tube connected to the mouth of the subject 180, a tracheostomy tube connected to the trachea of the subject 180. In some embodiments, the subject interface 170 may form a sealed connection with a face region of the subject 180 to facilitate the delivery of the respiratory gas at a pressure that has a sufficient variance with ambient pressure to effect therapy (e.g., a positive pressure of about 10 cmH₂O). For example, the subject interface 170 may be fixed to the nose of the subject 180 by various fixing ways (e.g., through a fixing rope or a fixing ring). In some embodiments, the subject interface 170 may not form a sealed connection with a face region of the subject 180 that is sufficient to facilitate delivery of the respiratory gas to the subject 180 at a positive pressure of about 10 cmH₂O. In some embodiments, the subject interface 170 may further include a filter configured to filter the respiratory gas. In some embodiments, the subject interface 170 may further include or be equipped with one or more sensors configured to detect parameters relating to the respiratory gas and/or the expired gas of the subject 180. In some embodiments, the subject interface 170 may further include or be equipped with one or more gas filter units configured to filter and/or purify the respiratory gas delivered to the subject 180. In some embodiments, the gas filter unit(s) (e.g., a coarse filter, a fine filter, or the like) may filter one or more particles in the respiratory gas. In some embodiments, the gas filter unit(s) may filter bacteria in the respiratory gas. In some embodiments, the gas filter unit(s) may filter pungent gas in the respiratory gas.

In some embodiments, the respiration tube 160 may be configured to guide the respiratory gas from the ventilatory treatment apparatus 110 to the subject interface 170. The respiration tube 160 may include a gas passage to guide the respiratory gas. In some embodiments, the respiration tube 160 may form a sealed connection with the gas export of the ventilatory treatment apparatus 110. In some embodiments, the respiration tube 160 may form a sealed connection with the subject interface 170. In some embodiments, the respiration tube 160 may further include a heater configured to heat the respiration tube 160, so that the respiratory gas flowing through the respiration tube 160 can be maintained at a certain temperature, preferably, at a temperature that human beings are comfortable with, such as, a temperature within 16-43° C., a temperature within 28-38° C. In some embodiments, the respiration tube 160 may further include or be equipped with one or more sensors configured to detect parameters relating to the respiratory gas and/or the expired gas of the subject 180. In some embodiments, the respiration tube 160 may further include or be equipped with one or more gas filter units configured to filter and/or purify the respiratory gas delivered to the subject 180. In some embodiments, the gas filter unit(s) (e.g., a coarse filter, a fine filter, or the like) may filter one or more particles in the respiratory gas. In some embodiments, the gas filter unit(s) may filter bacteria in the respiratory gas. In some embodiments, the gas filter unit(s) may filter pungent gas in the respiratory gas.

In some embodiments, the network 120 may include any suitable network that can facilitate the exchange of information and/or data for the system 100. In some embodiments, one or more components of the system 100 (e.g., the ventilatory treatment apparatus 110, the terminal 130, the processing device 140, or the storage device 150) may communicate information and/or data with one or more other components of the system 100 via the network 120. For example, the processing device 140 may obtain signals from the ventilatory treatment apparatus 110 via the network 120. As another example, the processing device 140 may obtain user instructions from the terminal 130 via the network 120. In some embodiments, the network 120 may be any type of wired or wireless network, or a combination thereof. The network 120 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network 120 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 120 may include one or more network access points. For example, the network 120 may include wired and/or wireless network access points such as base stations and/or internet exchange points through which one or more components of the system 100 may be connected to the network 120 to exchange data and/or information.

In some embodiments, the terminal 130 may include a mobile device 130-1, a tablet computer 130-2, a laptop computer 130-3, or the like, or any combination thereof. In some embodiments, the mobile device 130-1 may include a smart home device, a wearable device, a smart mobile device, a virtual reality device, an augmented reality device, or the like, or any combination thereof. In some embodiments, the smart home device may include a smart lighting device, a control device of an intelligent electrical apparatus, a smart monitoring device, a smart video camera, an interphone, or the like, or any combination thereof. In some embodiments, the wearable device may include a smart bracelet, smart footgear, a pair of smart glasses, a smart helmet, a smartwatch, smart clothing, a smart backpack, a smart accessory, or the like, or any combination thereof. In some embodiments, the smart mobile device may include a smartphone, a personal digital assistant (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include Google Glasses, an Oculus Rift, a Hololens, a Gear VR, etc. In some embodiments, the terminal 130 may remotely operate the ventilatory treatment apparatus 110. In some embodiments, the terminal 130 may operate the ventilatory treatment apparatus 110 via a wireless connection. In some embodiments, the terminal 130 may receive information and/or instructions inputted by a user, and send the received information and/or instructions to the ventilatory treatment apparatus 110 or to the processing device 140 via the network 120. In some embodiments, the terminal 130 may receive data and/or information from the processing device 140. In some embodiments, the terminal 130 may display information relating to the system 100. In some embodiments, the terminal 130 may be part of the processing device 140. In some embodiments, the terminal 130 may be omitted. In some embodiments, via the terminal 130, a user may remotely update software of the ventilatory treatment apparatus 110, and/or adjust or set one or more parameters of the ventilatory treatment apparatus 110.

In some embodiments, the processing device 140 may process data and/or information obtained from the ventilatory treatment apparatus 110, the terminal 130, and/or the storage device 150. For example, the processing device 140 may obtain signals detected by one or more sensors in the ventilatory treatment apparatus 110, the respiration tube 160, and/or the subject interface 170, and may process and/or analyze the signals to obtain one or more parameters relating to the respiratory gas, the expired gas of the subject 180, and/or the operation status of the ventilatory treatment apparatus 110. In some embodiments, the processing device 140 may be a single server, or a server group. The server group may be centralized, or distributed. In some embodiments, the processing device 140 may be local or remote. For example, the processing device 140 may access information and/or data stored in the ventilatory treatment apparatus 110, the terminal 130, and/or the storage device 150 via the network 120. As another example, the processing device 140 may be directly connected to the ventilatory treatment apparatus 110, the terminal 130, and/or the storage device 150 to access stored information and/or data. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing device 140 may be implemented on a computing device of the ventilatory treatment apparatus 110.

In some embodiments, the processing device 140 may include an acquisition unit and a processing unit. The acquisition unit may be configured to obtain information relating to the system 100 (e.g., the ventilatory treatment apparatus 110, the processing device 140, the storage device 150, the terminal 130, etc.). The information may include signals detected by the detection module, data read from the storage device 150, instructions or data provided by the terminal 130, etc. In some embodiments, the information may be transmitted to the processing unit for processing. In some embodiments, the acquisition unit may obtain or transmit the information via a tangible transmission media or a Carrier-wave transmission media. The tangible transmission media may include, for example, a coaxial cable, a copper wire, a fiber optics, or the like. The Carrier-wave transmission media may take the form of electric or electromagnetic signals (e.g., signals generated during radio frequency (RF) data communications). The processing unit may be configured to process the information obtained by the acquisition unit. The processing unit may include an advanced RISC machines processor (ARM), a programmable logic device (PLD), a microprogrammed control unit (MCU), a digital signal processor (DSP), a field-programmable gate array (FPGA), a system on chip (SoC) or the like, or any combination thereof.

In some embodiments, the storage device 150 may store data and/or instructions. In some embodiments, the storage device 150 may store data or information obtained from the ventilatory treatment apparatus 110. For example, the processing device 140 may determine one or more parameters relating to the respiratory gas, the expired gas of the subject 180, and/or the operation status of the ventilatory treatment apparatus 110 based on the signals obtained from one or more sensors of the ventilatory treatment apparatus 110, the respiration tube 160, and/or the subject interface 170. The determined parameter(s) may be stored in the storage device 150 for further use or processing. In some embodiments, the storage device 150 may store data obtained from the terminal 130 and/or the processing device 140. In some embodiments, the storage device 150 may store data and/or instructions that the processing device 140 may execute or use to perform exemplary methods described in the present disclosure. In some embodiments, the storage device 150 may include a mass storage device, removable storage device, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage device 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 150 may be connected to the network 120 to communicate with one or more components in the system 100 (e.g., the ventilatory treatment apparatus 110, the processing device 140, the terminal 130, etc.). One or more components in the system 100 may access the data or instructions stored in the storage device 150 via the network 120. In some embodiments, the storage device 150 may be directly connected to or communicate with one or more components in the system 100 (e.g., ventilatory treatment apparatus 110, the processing device 140, the terminal 130, etc.). In some embodiments, the storage device 150 may be part of the processing device 140. In some embodiments, the storage device 150 may be part of the ventilatory treatment apparatus 110.

FIGS. 2A-2F illustrate an exemplary ventilatory treatment apparatus according to some embodiments of the present disclosure. FIG. 2A shows a front side of the ventilatory treatment apparatus 200. FIG. 2B shows a rear side of the ventilatory treatment apparatus 200. FIG. 2C shows another rear side of the ventilatory treatment apparatus 200. FIG. 2D shows a rear view of the ventilatory treatment apparatus 200. FIG. 2E shows a side view of the ventilatory treatment apparatus 200. FIG. 2F shows a bottom view of the ventilatory treatment apparatus 200.

FIGS. 2G-2Q illustrate exemplary filter shells of an exemplary gas filter unit according to some embodiments of the present disclosure. FIGS. 2G-21 show an overall view, a side view, and a rear view of an exemplary filter shell, respectively. FIGS. 2J-2L show an overall view, a side view, and a rear view of another exemplary filter shell, respectively. FIGS. 2M-2Q show a side view, a front view, a rear view, and overall views of another exemplary filter shell, respectively.

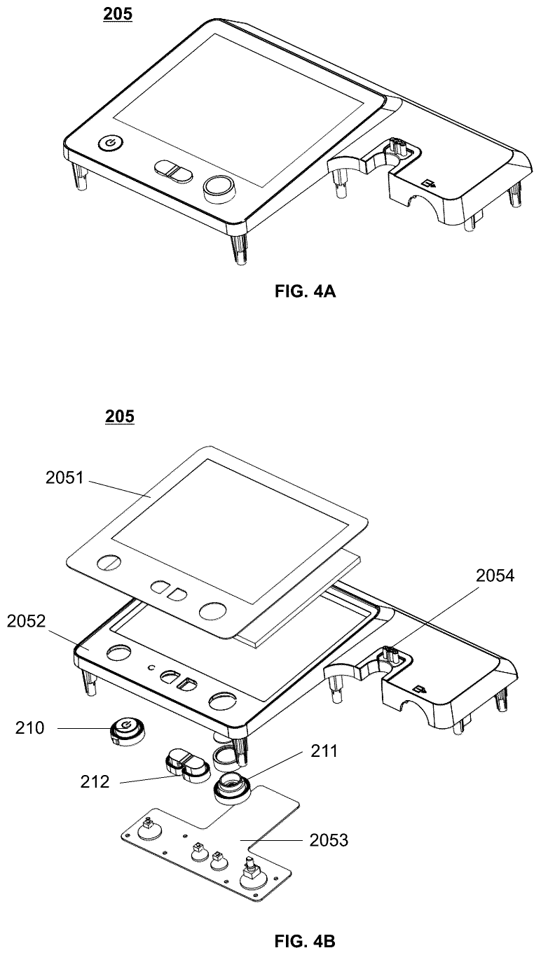

As illustrated in FIG. 2A, the ventilatory treatment apparatus 200 may include a main body 209 and a humidification assembly 206. The main body 209 of the ventilatory treatment apparatus 200 may include an on-off key 210, a displaying screen 205, a knob 211, one or more function buttons 212, or the like, disposed on an upper shell of the main body 209. The on-off key 210 may be configured to cause the ventilatory treatment apparatus 200 to switch between a boot state and a shutdown state. For example, if the ventilatory treatment apparatus 200 is switched off, a user (e.g., the subject 180) may press the on-off key 210 to boot the ventilatory treatment apparatus 200. As another example, if the ventilatory treatment apparatus 200 is switched on, the user (e.g., the subject 180) may press the on-off key 210 to shut down the ventilatory treatment apparatus 200. The displaying screen 205 may be configured to display information relating to the status of the ventilatory treatment apparatus 200, and/or information relating to the respiratory gas (e.g., a first gas, a second gas, and/or a mixed gas, etc.). The information displayed may include, for example, the parameters relating to the respiratory gas, the expired gas of the subject 180, and/or the operation status of the ventilatory treatment apparatus 200. More descriptions of the parameters may be found elsewhere in the present disclosure (e.g., FIG. 1 and the descriptions thereof). In some embodiments, the displaying screen 205 may be configured as a software operation interface of the ventilatory treatment apparatus 200. In some embodiments, the displaying screen 205 may be a touch panel. More descriptions of the displaying screen 205 may be found elsewhere in the present disclosure (e.g., FIGS. 4A-4B and descriptions thereof).

The knob 211 may be configured to facilitate a user (e.g., the subject 180) to adjust and/or set the value(s) of one or more parameters illustrated above and/or a menu item of software implemented in the ventilatory treatment apparatus 200. In some embodiments, the knob 211 may be turned and/or pressed. For example, the subject 180 may turn the knob 211 to adjust the value(s) of the pressure of the respiratory gas, the humidity of the respiratory gas, etc. As another example, the subject 180 may press the knob 211 to confirm an adjusted (or set) parameter, select a menu item, exit from a functional interface, etc. The function button(s) 212 may be pressed to switch to a main interface of the software, enter a selected functional interface, return to a previous interface, etc. In some embodiments, one or more of the function buttons 212 may be pressed to mute the hardware and/or software of the ventilatory treatment apparatus 200. In some embodiments, one or more of the on-off key 210, the displaying screen 205, the knob 211, and the one or more function buttons 212 may be set on the front side, the rear side, the top side, the left side, or the right side of the ventilatory treatment apparatus 200.

The ventilatory treatment apparatus 200 may include at least one gas entrance configured to introduce one or more gases into the apparatus. The one or more gases may include a first gas and/or a second gas. The first gas may include a target gas (e.g., oxygen). The second gas may include the target gas. In some embodiments, a second concentration ratio of the target gas in the second gas may exceed a first concentration ratio of the target gas in the first gas. In some embodiments, the target gas may include oxygen, the first gas may include the air, and the second gas may include high-concentration oxygen with higher concentration of oxygen than air. The second concentration ratio of the target gas in the second gas may be in a range from 21% to 100%.

In some embodiments, as shown in FIGS. 2B-2D, the at least one gas entrance may include a first gas entrance 201 and at least one second gas entrance 202. The first gas entrance 201 may be configured to introduce the first gas into the apparatus. The at least one second gas entrance 202 may be configured to introduce the second gas into the apparatus. In some embodiments, the at least one gas entrance (e.g., the at least one second gas entrance 202) may include a first-second gas entrance 2021 and a second-second gas entrance 2022. The first-second gas entrance 2021 may be configured to introduce the second gas from a first gas source into the apparatus. The second-second gas entrance 2022 may be configured to introduce the second gas from a second gas source into the apparatus. The first gas source may include an oxygenerator, a wall oxygen source, etc. The second gas source may include an oxygenerator, an oxygen cylinder filled with oxygen, etc. In some embodiments, the first gas source and the second gas source may be two different gas sources or two gas sources that are the same. In some embodiments, the first gas source and the second gas source may share a single gas source. An exemplary first or second gas source may be found in FIG. 24. In some embodiments, the first-second gas entrance 2021 and the second-second gas entrance 2022 may be configured as a single gas entrance. In some embodiments, the first gas entrance 201 and the at least one second gas entrance 202 may be configured as a single gas entrance.

In some embodiments, the ventilatory treatment apparatus 200 may include a gas mixing assembly (not shown in FIGS. 2A-2F) configured to mix the first gas and the second gas to generate a mixed gas. An exemplary gas mixing assembly may be shown in FIGS. 7B-7D. In some embodiments, as shown in FIGS. 2A-2E, the ventilatory treatment apparatus 200 may include a gas export 204 configured to discharge a flow of the mixed gas, for example, to the respiration tube 160. In some embodiments, the ventilatory treatment apparatus 200 may include a noise reduction assembly (not shown in FIGS. 2A-2F) configured to reduce noise(s) of the apparatus (e.g., a noise generated by a gas pressurization unit when pressurizing the first gas or the mixed gas). An exemplary noise reduction assembly may be shown in FIGS. 25A-27B, 30, and 36A-36C. In some embodiments, the gas pressurization unit may be configured to generate a pressurized first gas by pressurizing the first gas before the gas mixing assembly mixes the first gas and the second gas. Alternatively, the gas pressurization unit may be configured to generate a pressurized mixed gas by pressurizing the mixed gas after the gas mixing assembly mixes the first gas and the second gas.

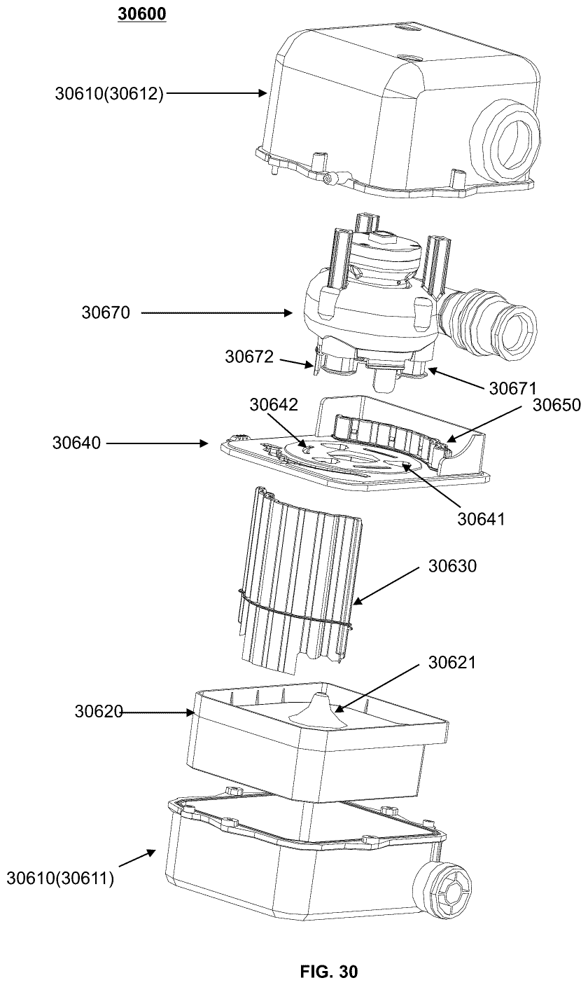

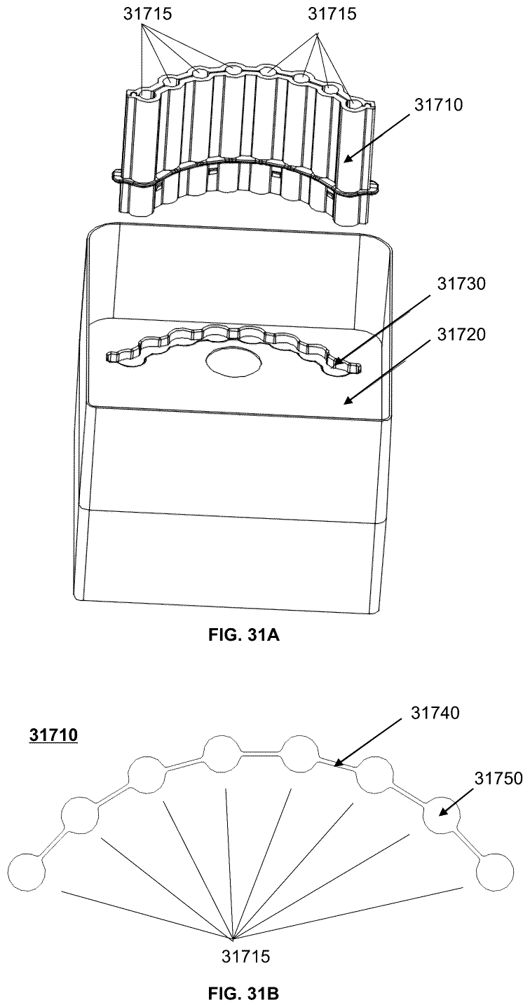

In some embodiments, the gas pressurization unit (e.g., the gas pressurization unit 30670 shown in FIG. 30) may include a gas inlet port and a gas outlet port. In some embodiments, the gas inlet port of the gas pressurization unit may be in fluid communication with the at least one gas entrance (e.g., via a tube) to receive the first gas from the at least one gas entrance (e.g., the first gas entrance 201). In some embodiments, the gas outlet port of the gas pressurization unit may be in fluid communication with an inlet port (e.g., a first inlet port) of the gas mixing assembly to introduce the pressurized first gas into the gas mixing assembly through the first inlet port of the gas mixing assembly.

Alternatively, in some embodiments, the gas mixing assembly may include at least one gas inlet port (e.g., a first gas inlet port 731 shown in FIGS. 7B and 7D, a second gas inlet port 732 shown in FIGS. 7B and 7D) and a gas outlet port (not shown). The at least one gas inlet port of the gas mixing assembly may be in fluid communication with the at least one gas entrance (e.g., via tube(s)) to receive the first gas and the second gas from the at least one gas entrance. The gas outlet port of the gas mixing assembly may be in fluid communication with the gas inlet port of the gas pressurization unit to introduce the mixed gas into the gas pressurization unit, and the gas pressurization unit may pressurize the mixed gas.

In some embodiments, the humidification assembly 206 may be configured to humidify (and/or warm) the mixed gas discharged from the gas mixing assembly or humidify (and/or warm) the pressurized mixed gas discharged from the gas pressurization unit. The humidification assembly 206 may be removably coupled to the main body 209 of the ventilatory treatment apparatus 200. The humidification assembly 206 may be in fluid communication with a gas outlet port 207 of the main body 209 and the gas export 204 of the ventilatory treatment apparatus 200. The gas outlet port 207 of the main body 209 may be in fluid communication with the gas outlet port of the gas mixing assembly or the gas outlet port of the gas pressurization unit. The gas export 204 may be in fluid communication with the respiration tube 160. The humidification assembly 206 may receive the (pressurized) mixed gas from the gas outlet port 207 of the main body 209, humidify (and/or warm) the (pressurized) mixed gas, and discharge the humidified (and/or) warmed respiratory gas into the respiration tube 160 via the gas export 204 of the ventilatory treatment apparatus 200. An exemplary humidification assembly may be shown in FIGS. 15, 22, 37A-40B.

In some embodiments, the ventilatory treatment apparatus 200 may include a gas importing mechanism (not shown in FIGS. 2A-2F) configured to import the second gas, from at least one of a first gas source and a second gas source, via a gas outlet port of the gas importing mechanism and an inlet port (e.g., a second inlet port 732 shown in FIGS. 7B and 7D) of the gas mixing assembly, into the gas mixing assembly. An exemplary gas importing mechanism may be shown in FIGS. 7A-7E.

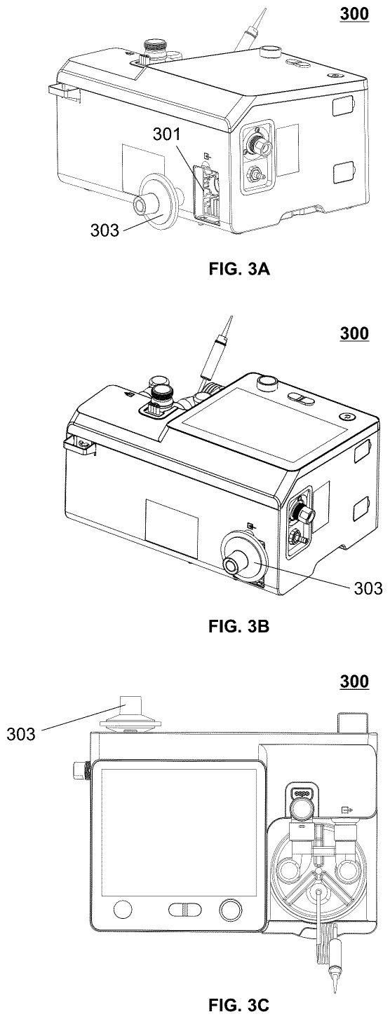

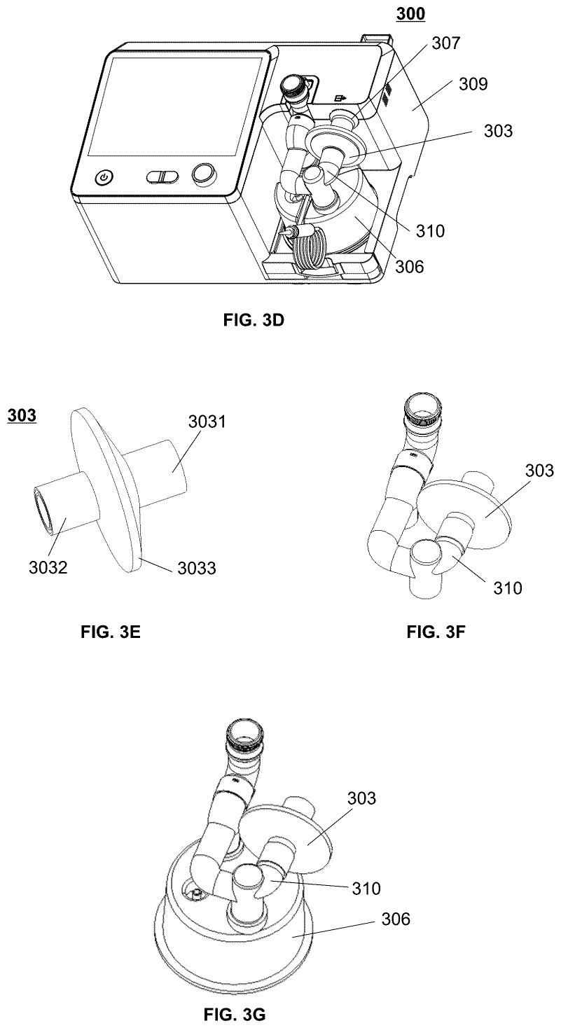

In some embodiments, the ventilatory treatment apparatus 200 may include or be equipped with one or more gas filter units configured to filter and/or purify the respiratory gas delivered to the subject 180. In some embodiments, the gas filter unit(s) (e.g., a coarse filter, a fine filter, or the like) may filter one or more particles in the respiratory gas. In some embodiments, the gas filter unit(s) may filter bacteria in the respiratory gas. In some embodiments, the gas filter unit(s) may filter pungent gas in the respiratory gas. Merely by way of example, the ventilatory treatment apparatus 200 may include or be equipped with a gas filter unit 203. The gas filter unit 203 may be configured to filter particle(s), bacteria, or pungent gas, or the like, in the first gas entering the apparatus (e.g., via the first gas entrance 201). The gas filter unit 203 may be disposed at the first gas entrance 201. In some embodiments, the gas filter unit 203 may include a filter cotton 2032 and a filter shell 2031. The filter cotton 2032 may be configured to filter particle(s), bacteria, or pungent gas, or the like, in the first gas entering the apparatus (e.g., via the first gas entrance 201). The filter shell 2031 may be configured to fix the filter cotton 2032 at the first gas entrance 201, and/or protect the filter cotton 2032 from being damaged. As shown in FIGS. 2G-2L, the filter shell 2031 may include an inner side 20311 facing the filter cotton 2032 and an outer side 20312 facing the outside of the ventilatory treatment apparatus 200. In some embodiments, the outer side 20312 of the filter shell 2031 may have a special-shaped surface (e.g., an arc-shaped surface shown in FIGS. 2H and 2K). In some embodiments, the outer side 20312 of the filter shell 2031 may have one or more protruding parts (e.g., protruding strip(s) 20313 shown in FIG. 2L, or protruding parts 20314 shown in FIGS. 2M-2N and 2P-2Q). In some embodiments, one or more of the protruding part(s) may have gradual changing height(s). In some embodiments, an enveloping surface of the protruding part(s) may be a curved surface. Additionally or alternatively, the outer side of the filter shell 2031 may have an unequal-height design. For example, one or more portions of the outer side may be raised or sunken with respect to the remaining portion(s) of the outer side. As another example, an enveloping surface of one or more raised portions of the outer side may be a curved surface. Therefore, the special-shaped surface of the outer side of the filter shell 2031 may prevent foreign matter(s) from being adsorbed or attached on the outer side and blocking the first gas entering the ventilatory treatment apparatus 200 via the first gas entrance 201. In some embodiments, the filter shell 2031 mounted on the first gas entrance 201 may have various shapes (see FIGS. 2G-2Q), and/or the placement direction of the filter shell 2031 may be various (e.g., the filter shell 2031 in FIGS. 2M-2Q may be placed horizontally, the filter shell 2031 in FIGS. 2G-2L may be placed vertically). Another exemplary gas filter unit may be shown in FIGS. 3A-3G.

In some embodiments, as shown in FIG. 2F, the ventilatory treatment apparatus 200 may include one or more holes 208 set on a baseplate 220 of the main body 209. The one or more holes 208 may be configured to drain a certain amount of liquids leaking from the humidification assembly 206. In some embodiments, during adding liquids into the humidification assembly 206 or in other situations (e.g., if the ventilatory treatment apparatus 200 is placed obliquely (i.e., the humidification assembly 206 is placed obliquely), or the humidification assembly 206 is untightly sealed), a certain amount of liquids may leak from the humidification assembly 206 and onto the baseplate 220 below the humidification assembly 206. The leaked liquids may flow out of the ventilatory treatment apparatus 200 through the holes 208. Therefore, the leaked liquids may not accumulate on the baseplate 220. In some embodiments, the cross section of each of the one or more holes 208 may have a step shape. In some embodiments, the holes 208 may facilitate the draining of the leaked liquids. In some embodiments, the holes 208 may prevent a foreign matter (e.g., a finger of the subject 180) from entering the ventilatory treatment apparatus 200. In some embodiments, the one or more holes 208 may conform to international standards to make the overall appearance of the ventilatory treatment apparatus 200 more elegant and/or to prevent the subject 180 from directly looking into the internal space of the ventilatory treatment apparatus 200 from the outside.

FIGS. 3A-3G illustrate an exemplary gas filter unit according to some embodiments of the present disclosure. FIG. 3A shows a rear side of a ventilatory treatment apparatus 300 and an unmounted gas filter unit 303. FIG. 3B shows a rear side of the ventilatory treatment apparatus 300 equipped with the gas filter unit 303. FIG. 3C shows a top view of the ventilatory treatment apparatus 300 equipped with the gas filter unit 303. FIG. 3D shows the gas filter unit 303 mounted on a humidification assembly 306 of the ventilatory treatment apparatus 300. FIG. 3E shows a side view of the gas filter unit 303. FIGS. 3F and 3G shows the gas filter unit 303 connected with a gas transmission interface 310 of the humidification assembly 306.

The ventilatory treatment apparatus 300 may be similar to or the same as the ventilatory treatment apparatus 200 shown in FIGS. 2A-2F, except that the gas filter unit 303 of the ventilatory treatment apparatus 300 is different from the gas filter unit 203 of the ventilatory treatment apparatus 200. As shown in FIG. 3E, the gas filter unit 303 may include a gas inlet port 3031, a gas outlet port 3032, and a filter cartridge 3033. The gas inlet port 3031 may be configured to introduce the respiratory gas (e.g., the first gas, the mixed gas) into the filter cartridge 3033. The filter cartridge 3033 may be configured to filter bacteria, and/or viruses in the respiratory gas to generate a filtered gas. The gas outlet port 3032 may be configured to export the filtered gas.

In some embodiments, as shown in FIGS. 3A-3C, the gas filter unit 303 may be disposed at a first gas entrance 301 of the ventilatory treatment apparatus 300 to filter the first gas to generate a filtered first gas. The first gas entrance 301 of the ventilatory treatment apparatus 300 may be similar to or the same as the first gas entrance 201 of the ventilatory treatment apparatus 200.

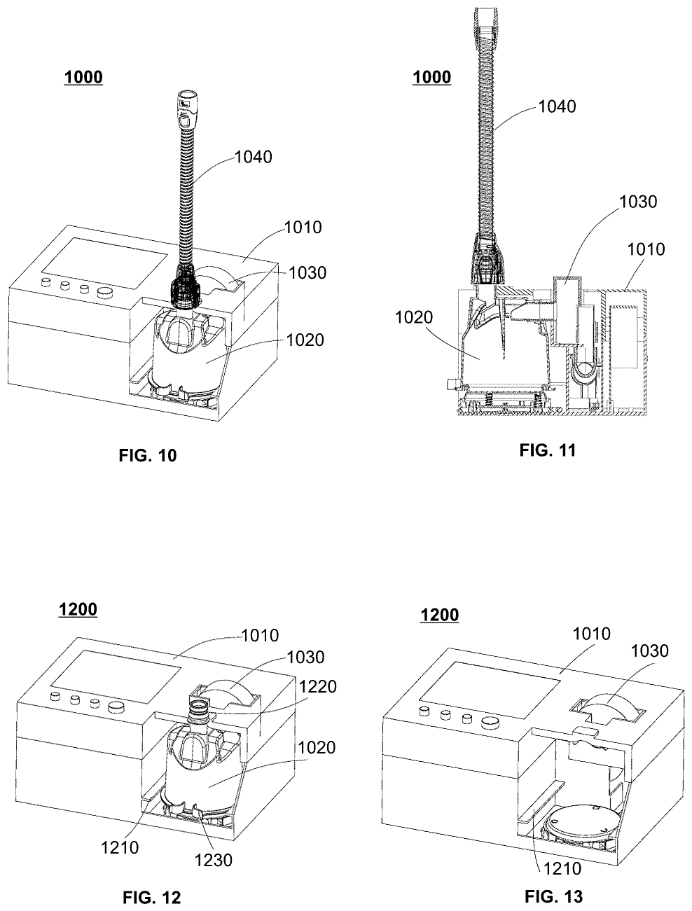

Alternatively, in some embodiments, as shown in FIGS. 3D, 3F and 3G, the gas filter unit 303 may be disposed on the humidification assembly 306. For example, the gas filter unit 303 may be disposed between the main body 309 of the ventilatory treatment apparatus 300 and the humidification assembly 306. Specifically, the gas filter unit 303 may be connected with the gas transmission interface 310 of the humidification assembly 306, the gas inlet port 3031 of the gas filter unit 303 may be connected to the gas outlet port 307 of the main body 309 of the ventilatory treatment apparatus 300, and the gas outlet port 3032 of the gas filter unit 303 may be connected to the gas transmission interface 310. The gas outlet port 307 of the main body 309 of the ventilatory treatment apparatus 300 may be similar to or the same as the gas outlet port 207 of the main body 209 of the ventilatory treatment apparatus 200. In such cases, the gas filter unit 303 may filter the mixed gas (discharged from the main body 309) to generate a filtered mixed gas, and the filtered mixed gas may be humidified (and/or warmed) by the humidification assembly 306. The gas transmission interface 310 may be configured to facilitate a gas communication between a tank body of the humidification assembly 306 with the ventilatory treatment apparatus 300 and/or the respiration tube 160. In some embodiments, the humidification assembly 306 may be replaced by the humidification assembly shown in FIGS. 15, 22, and 37A-40B. In some embodiments, the gas filter unit(s) may be disposed at other positions of the ventilatory treatment apparatus (see FIGS. 10-14 and 18-21). Other exemplary gas filter unit(s) may be found elsewhere in the present disclosure (e.g., FIGS. 16-17 and 23).

FIGS. 4A-4B illustrate an exemplary displaying screen according to some embodiments of the present disclosure. FIG. 4A shows an overall view of the displaying screen 205 of the ventilatory treatment apparatus 200. FIG. 4B shows an exploded view of the displaying screen 205.

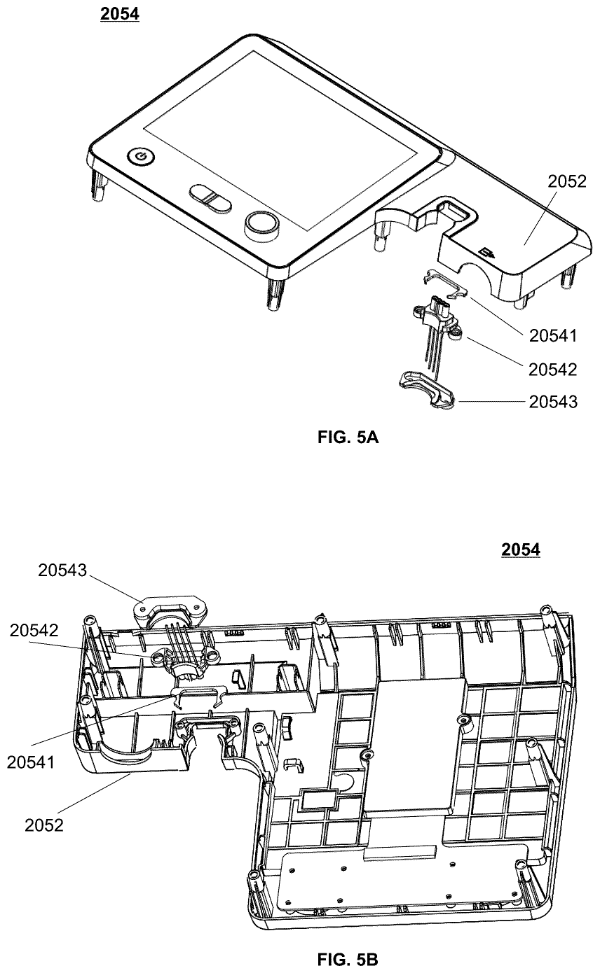

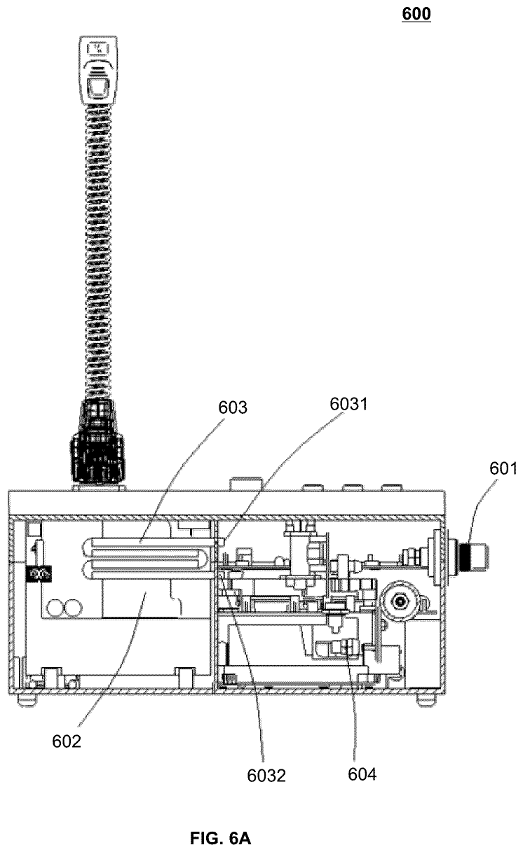

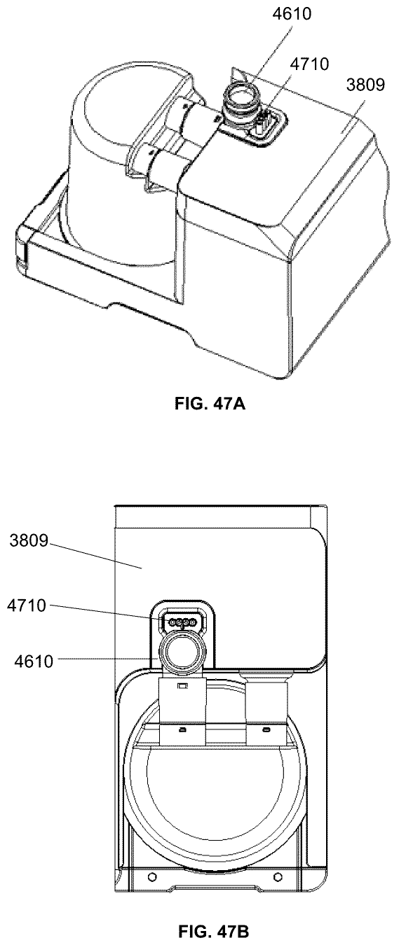

As shown in FIG. 4B, the displaying screen 205 may include a display panel 2051, an upper shell 2052 of the main body 209, and a circuit board 2053. The display panel 2051 may include a liquid crystal display panel, a touch panel, etc. The display panel 2051 may be imbedded into or integrated into the upper shell 2052 of the main body 209. The on-off key 210, the knob 211, and one or more function buttons 212 may be imbedded into or integrated into the upper shell 2052 of the main body 209. The circuit board 2053 may include one or more circuits configured to provide power to the display panel 2051, and control the working status of the display panel 2051. In some embodiments, the upper shell 2052 of the main body 209 may further include or be equipped with an electrical interface 2054 of the ventilatory treatment apparatus 200. The electrical interface 2054 may be configured to provide an electrical connection between the ventilatory treatment apparatus 200 and the respiration tube 160. More descriptions of the electrical interface 2054 may be found elsewhere in the present disclosure (e.g., FIGS. 5A-5B, and 47A-47B and descriptions thereof).

FIGS. 5A-5B illustrate an exemplary electrical interface of the ventilatory treatment apparatus according to some embodiments of the present disclosure. FIG. 5A shows an exploded view of a front side of the electrical interface 2054 of the ventilatory treatment apparatus 200. FIG. 5B shows an exploded view of a rear side of the electrical interface 2054 of the ventilatory treatment apparatus 200.

As shown in FIGS. 5A-5B, the electrical interface 2054 may be imbedded into or integrated into the upper shell 2052 of the main body 209. In some embodiments, the electrical interface 2054 may be removably coupled to the upper shell 2052. The electrical interface 2054 may include a gasket 20541, a socket 20542, and a binder plate 20543. The socket 20542 may be configured to provide an electrical connection between the ventilatory treatment apparatus 200 and the respiration tube 160. The gasket 20541 may be configured to facilitate a secure connection between the socket 20542 and the upper shell 2052. The gasket 20541 may include a silicone pad. The binder plate 20543 may be configured to press the socket 20542 to the upper shell 2052. In some embodiments, the binder plate 20543, the socket 20542 and/or the gasket 20541 may be fixed to the upper shell 2052 with one or more screws.

FIGS. 6A-6C illustrate an exemplary ventilatory treatment apparatus with a radiating tube according to some embodiments of the present disclosure. FIG. 6A shows a section view of the ventilatory treatment apparatus 600. FIG. 6B shows a section view of the ventilatory treatment apparatus 600 from a left perspective. FIG. 6C shows a section view of the ventilatory treatment apparatus 600 from an overlooking perspective. The ventilatory treatment apparatus 600 may be similar to or the same as the ventilatory treatment apparatus 200 shown in FIGS. 2A-2F.

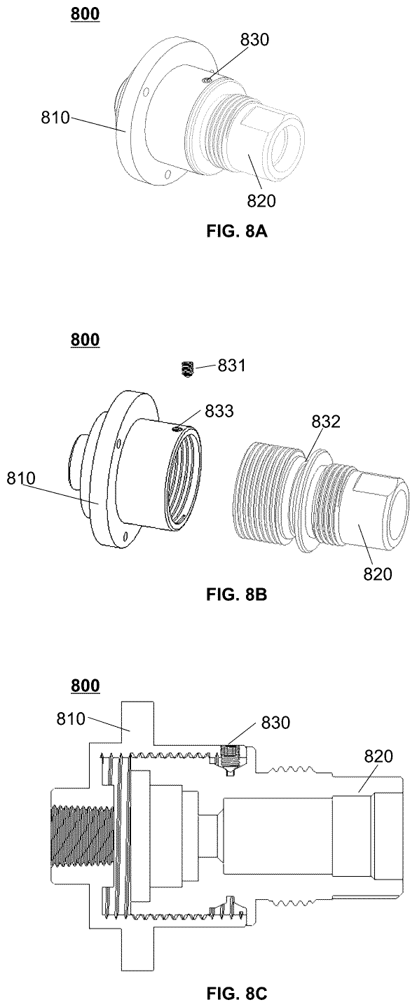

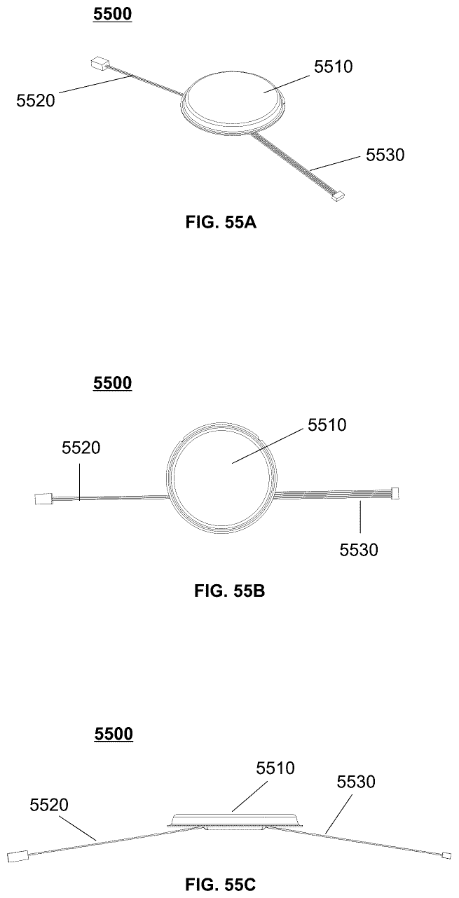

As shown in FIGS. 6A-6C, the ventilatory treatment apparatus 600 may include at least one second gas entrance. The ventilatory treatment apparatus 600 may include one or more gas passages in fluid communication with the at least one second gas entrance. The ventilatory treatment apparatus 600 may include a heating component configured to heat the respiratory gas flowing in the gas passage(s), and a power supply component 602 (e.g., a non-isolated power supply component 60130 shown in FIG. 60) electrically connected to the heating component. In some embodiments, the ventilatory treatment apparatus 600 may include a gas intake connector assembly 601 disposed at the at least one second gas entrance. The gas intake connector assembly 601 may be configured to introduce the respiratory gas (e.g., the second gas) from at least one gas source (e.g., an oxygenerator, a wall oxygen source, an oxygen cylinder filled with oxygen, etc.), via the at least one second gas entrance, into the ventilatory treatment apparatus 600. Therefore, the second gas discharged from the at least one gas source may be introduced into the gas passage(s) through the gas intake connector assembly 601. More descriptions of the gas intake connector assembly may be found elsewhere in the present disclosure (e.g., the gas intake connector assembly 800 in FIGS. 8A-8C and descriptions thereof). The gas passage(s) may go through a humidification assembly (e.g., the humidification assembly 206 shown in FIG. 2A) and/or a proportional valve 604 configured to control a flow of the respiratory gas introduced from the at least one gas source. The heating component may include a heater assembly (e.g., the heater assembly 5500 shown in FIGS. 55A-55C) configured to heat a liquid in the humidification assembly. The thermal power for heating the liquid may be provided by the power supply component 602.

In some embodiments, heat may be generated in the working process of the power supply component 602, and the heat may need to be dissipated in time. Therefore, in some embodiments, the ventilatory treatment apparatus 600 may include or be equipped with a radiating tube 603 for dissipating the heat. In some embodiments, the radiating tube 603 may be disposed at one or more segments of the gas passage(s). In some embodiments, the radiating tube 603 may be configured as a portion of the gas passage(s). In some embodiments, the radiating tube 603 may be disposed adjacent to the power supply component 602 or pass through the power supply component 602. Therefore, the gas flowing through the radiating tube 603 may take the heat away from the power supply component 602, which may decrease the temperature of the power supply component 602 and make the power supply component 602 work in a certain temperature range, thereby improving the work efficiency and stability of the power supply component 602, improving the service life and stability of the ventilatory treatment apparatus 600. In addition, the temperature of the gas flowing through the radiating tube 603 may be increased, thereby reducing the thermal power provided by the power supply component 602. In some embodiments, the power supply component 602 may provide thermal power for the humidification assembly. Additionally or alternatively, the power supply component 602 may provide power for one or more other components of the ventilatory treatment apparatus 600 (e.g., a displaying screen of the ventilatory treatment apparatus 600).

In some embodiments, the power supply component 602 may include a power circuit board and a cooling fin in connection with the power circuit board. The cooling fin may be configured to dissipate the heat of the power circuit board. In such cases, the radiating tube 603 may be disposed adjacent to the cooling fin of the power supply component 602 to take away the heat of the power circuit board in time, and guarantee normal working of the power supply component 602.

In some embodiments, the radiating tube 603 may be made of a material different from that of the remaining parts of the gas passage(s). The radiating tube 603 may have a reasonable size so as to facilitate the gas flowing through the radiating tube 603 take away the heat of the power supply component 602. In some embodiments, the gas intake connector assembly 601 may be connected to a gas inlet port 6031 of the radiating tube 603 through a tube made of polyurethane (PU). The tube made of polyurethane may have a relatively high rigidity that is sufficient to bear a relatively high pressure (e.g., 1.4 MPa) of the gas entering the gas intake connector assembly 601. It should be noted that the tube connecting the gas inlet port 6031 of the radiating tube 603 and the gas intake connector assembly 601 (and a tube connecting a gas outlet port 6032 of the radiating tube 603) is not shown in FIG. 6A, so as to show the relative positions of the radiating tube 603 and the power supply component 602.

In some embodiments, the gas passage(s) subsequent to the gas outlet port 6032 of the radiating tube 603 may include or be equipped with the proportional valve 604. An opening degree of the proportional valve 604 may be controlled to control a concentration ratio of the target gas (e.g., oxygen) in the respiratory gas. The proportional valve 604 may be connected with the gas outlet port 6032 of the radiating tube 603 through a tube (e.g., a silicone hose). In some embodiments, the proportional valve 604 may be removed.

In some embodiments, the radiating tube 603 may be disposed above a dissipation region (e.g., a region of the cooling fin) of the power supply component 602. In some embodiments, an elongated region of the radiating tube 603 may be the same as or larger than the dissipation region. In some embodiments, the radiating tube 603 may elongate back and forth between the gas inlet port 6031 and the gas outlet port 6032 to improve cooling efficiency. In some embodiments, the smaller the diameter of the radiating tube 603 is, and the radiating tube 603 is arranged more densely, the higher the cooling efficiency is. In some embodiments, an inner diameter of the radiating tube 603 may be in a range from 1 mm to 10 mm (e.g., 4 mm).

In some embodiments, the radiating tube 603 may be made of a metal (e.g., aluminum, copper, etc.). Therefore, the radiating tube 603 may have a relatively high sealing performance. A thickness of the radiating tube 603 may be relatively small (e.g., in a range from 0.1 mm to 2 mm).

FIGS. 7A-7E illustrate an exemplary gas importing mechanism according to some embodiments of the present disclosure. FIG. 7A shows a section view of a ventilatory treatment apparatus 700 including the gas importing mechanism 710 from a top perspective. FIG. 7B shows a top view of the gas importing mechanism 710. FIG. 7C shows a rear side view of the gas importing mechanism 710. FIG. 7D shows an axonometric drawing of the gas importing mechanism 710. FIG. 7E shows a schematic diagram of the gas importing mechanism 710.

In some embodiments, as shown in FIGS. 7B-7D, the first gas may enter, via the first gas entrance 701, and the gas inlet port 721 of the gas pressurization unit 720, into the gas pressurization unit 720. The first gas may be pressurized by the gas pressurization unit 720 to generate a pressurized first gas. The (pressurized) first gas may be discharged from the gas outlet port 722 of the gas pressurization unit 720 and flow into the gas mixing assembly 730 via the first gas inlet port 731 of the gas mixing assembly 730. The second gas may enter, via the first-second gas entrance 7021, and the first gas inlet port 711 of the gas importing mechanism 710, into the gas importing mechanism 710, which is illustrated by the solid arrows in FIGS. 7C and 7D. Additionally or alternatively, in some embodiments, the second gas may enter, via the second-second gas entrance 7022, and the second gas inlet port 712 of the gas importing mechanism 710, into the gas importing mechanism 710, which is illustrated by the dotted arrows in FIGS. 7C and 7D. The second gas may be discharged from the gas outlet port 713 of the gas importing mechanism 710 and flow into the gas mixing assembly 730 via the second gas inlet port 732 of the gas mixing assembly 730, which is illustrated by the dash dot arrows in FIGS. 7B and 7D. The (pressurized) first gas and the second gas may be mixed in and/or by the gas mixing assembly 730 to generated a mixed gas. The mixed gas may be discharged from the gas mixing assembly 730 and then discharged via the gas outlet port 707 of the main body 709 of the ventilatory treatment apparatus 700.

In some embodiments, the gas mixing assembly 730 may receive (via the first gas inlet port 731 of the gas mixing assembly 730) the pressurized first gas from a gas outlet port 722 of the gas pressurization unit 720, and receive (via the second gas inlet port 732 of the gas mixing assembly 730) the second gas from the gas outlet port 713 of the gas importing mechanism 710 (see the dash dot arrows illustrated in FIGS. 7B and 7D).

Alternatively, in some embodiments, the gas mixing assembly 730 may receive the first gas from at least one gas entrance (e.g., the first gas entrance 701), and receive the second gas from the gas outlet port 713 of the gas importing mechanism 710, before the gas pressurization unit 720 pressurizes the mixed gas (of the first gas and the second gas), which is not shown in FIGS. 7A-7D.

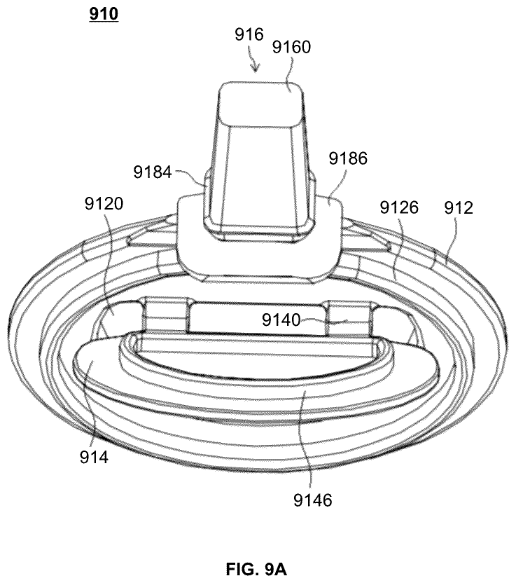

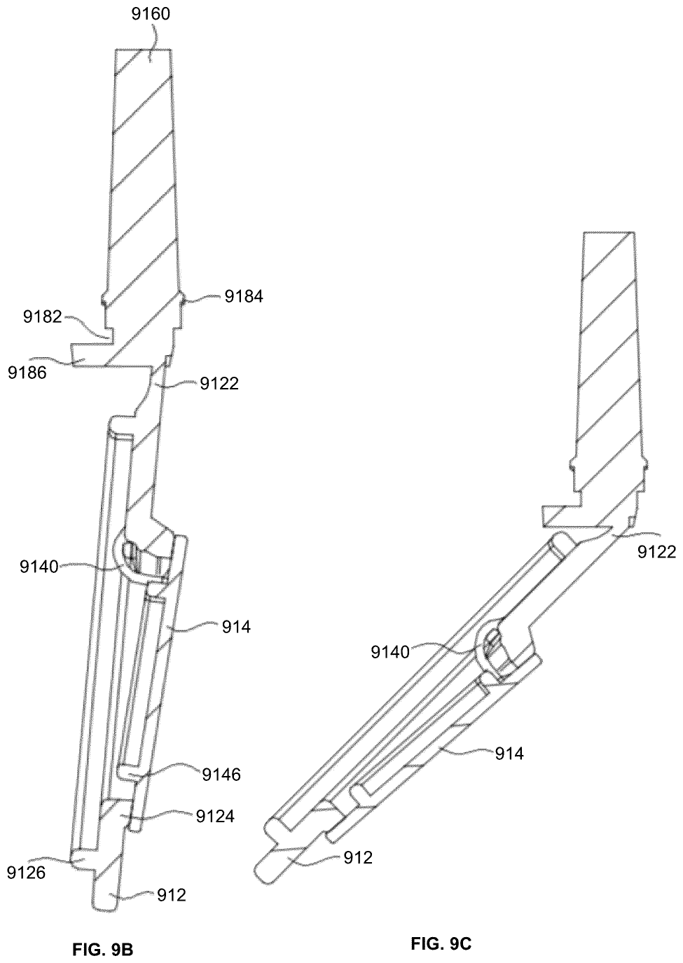

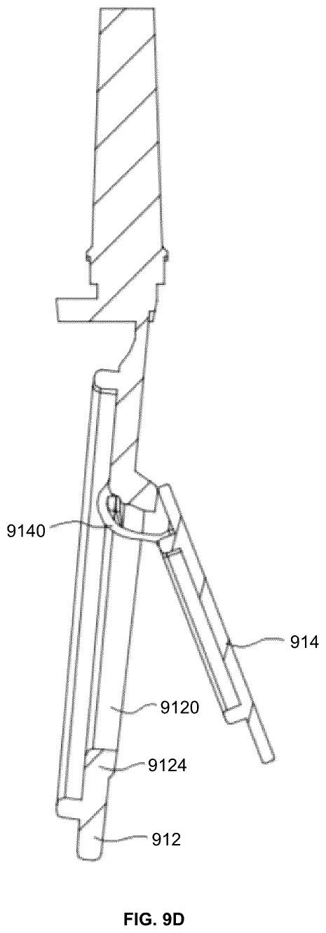

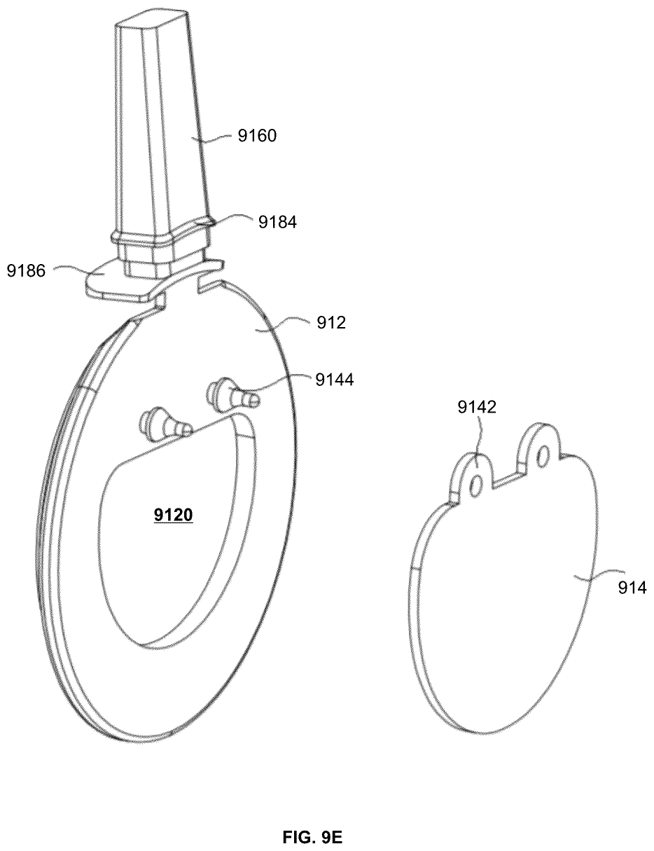

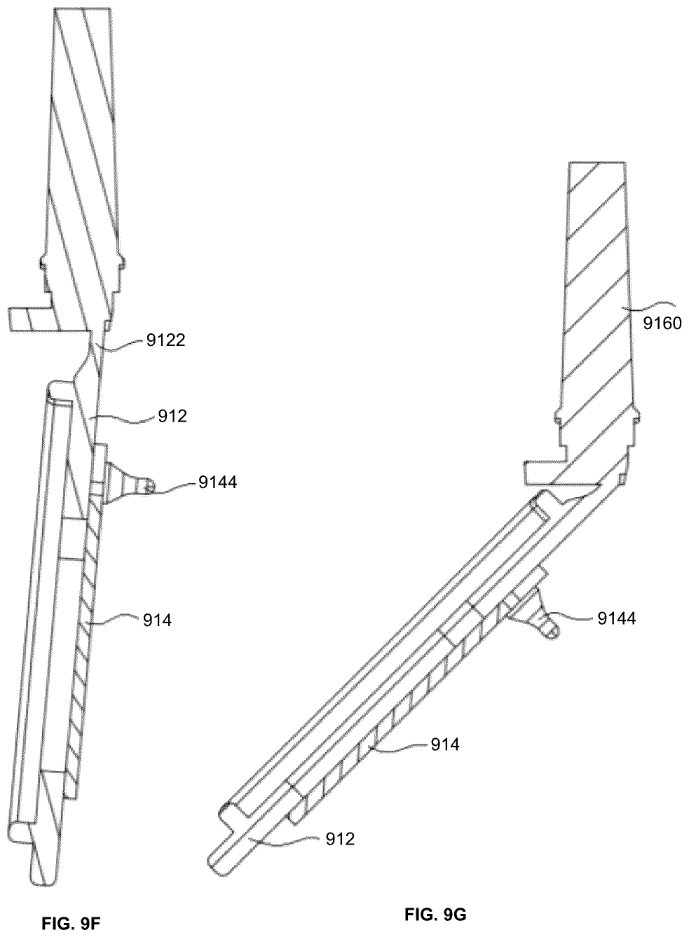

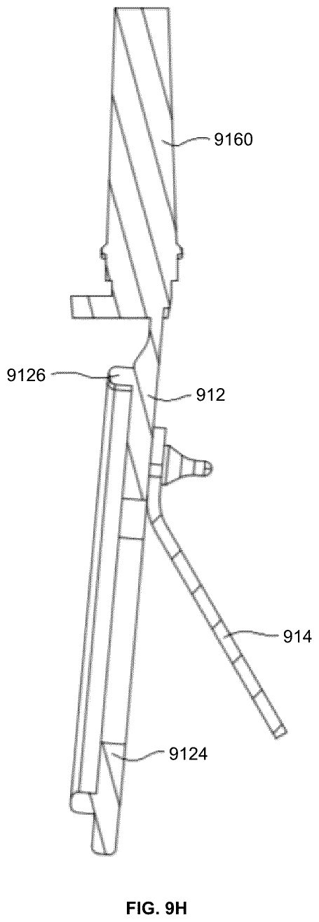

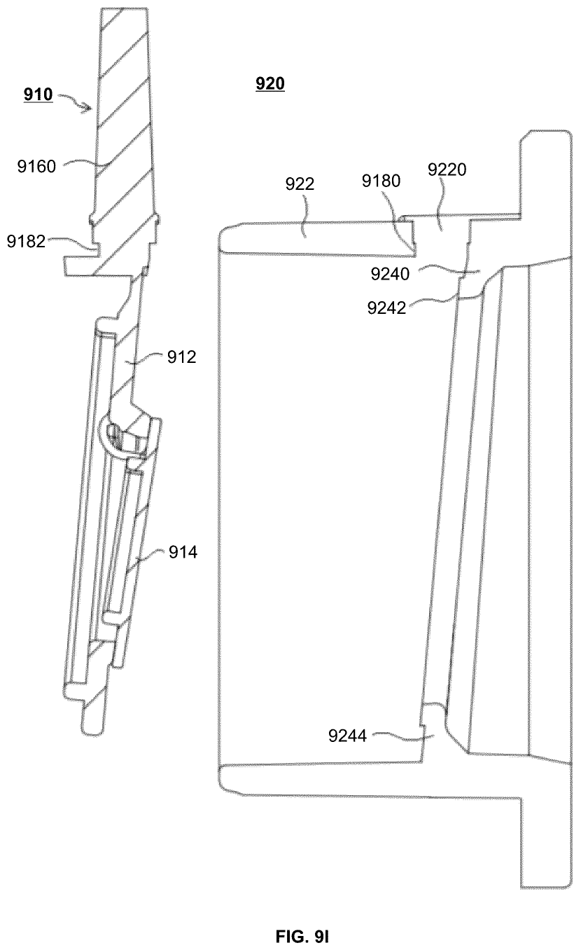

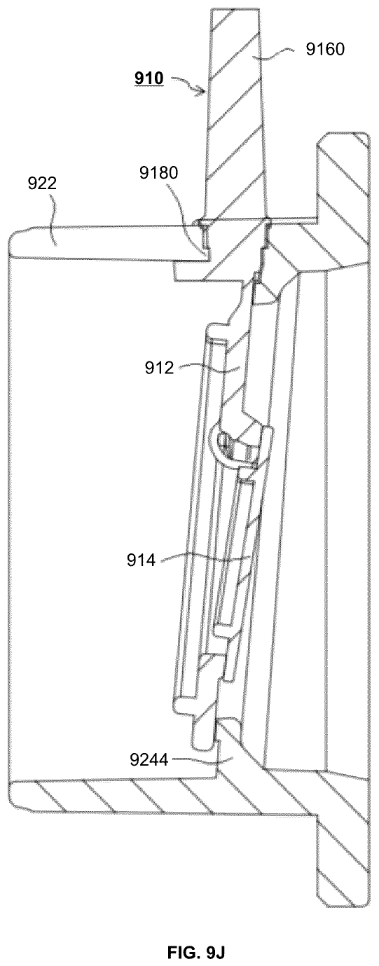

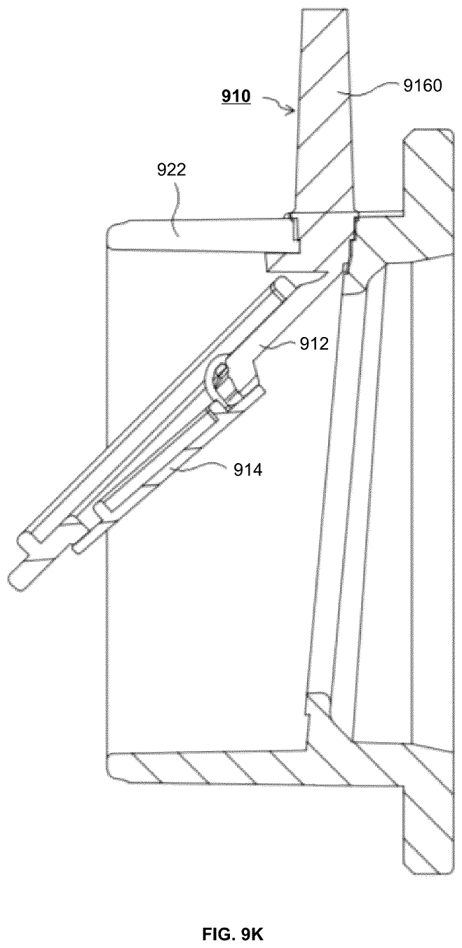

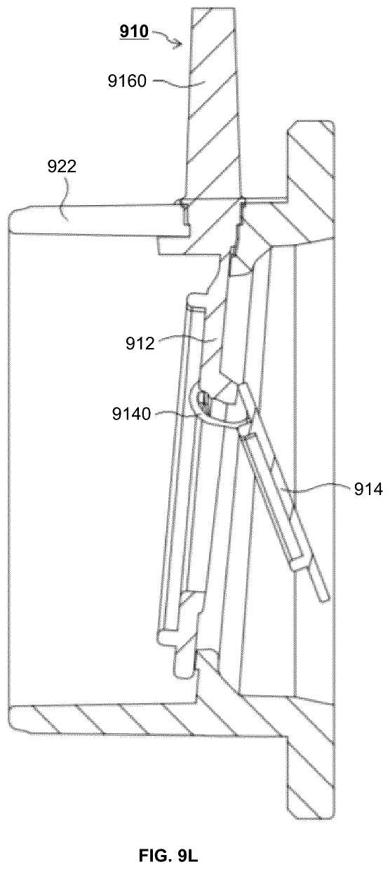

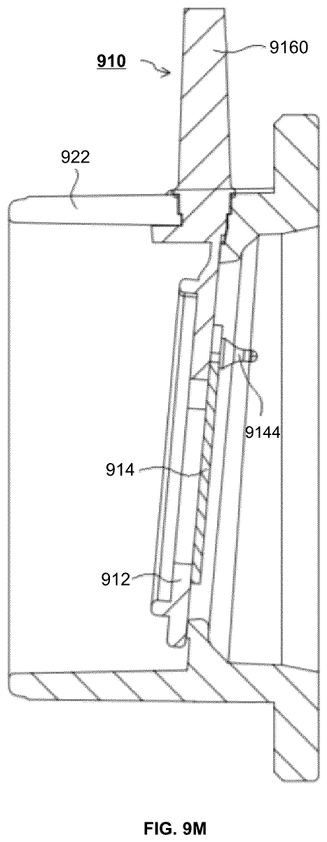

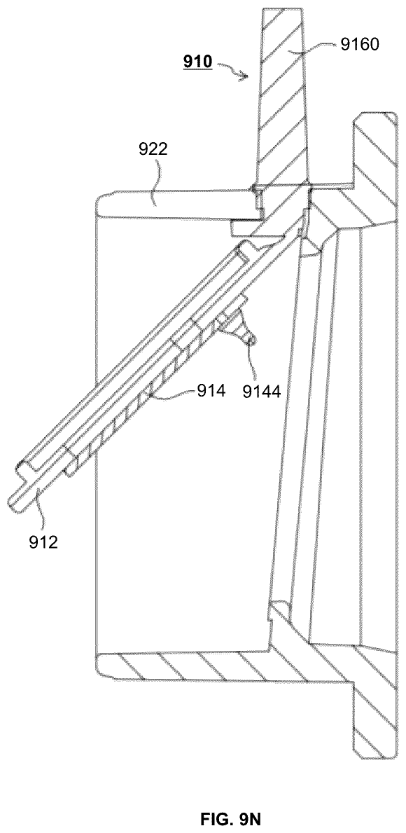

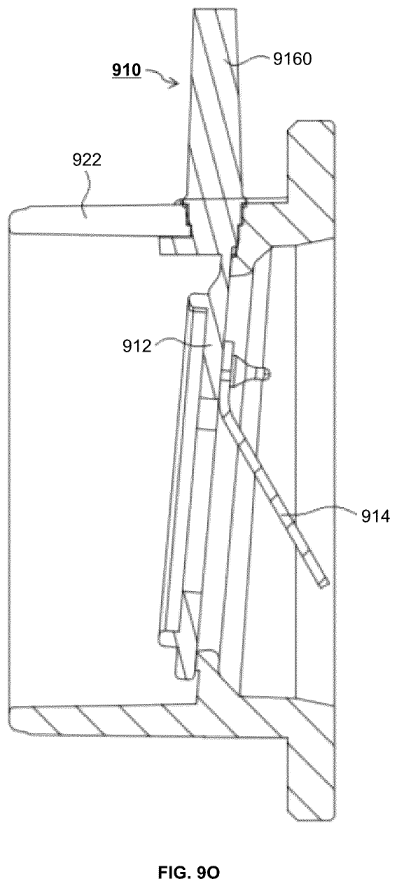

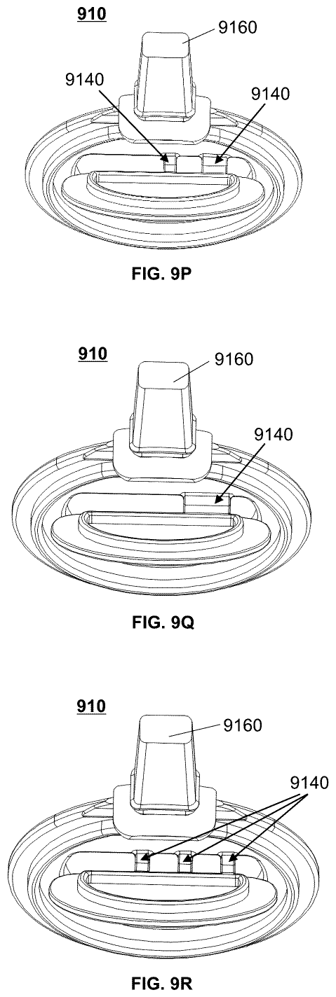

In some embodiments, as shown in FIGS. 7C-7E, the gas importing mechanism 710 may include a first sub-mechanism 750 and/or a second sub-mechanism 780. The first sub-mechanism 750 may be configured to import the second gas from the first gas source 741 (via the first-second gas entrance 7021) automatically. The second sub-mechanism 780 may be configured to import the second gas from the second gas source 742 (via the second-second gas entrance 7022) manually. In some embodiments, as shown in FIG. 7E, the first sub-mechanism 750 may include a pressure relief valve 752 configured to lower a pressure of the second gas introduced from the first gas source 741 (via the first-second gas entrance 7021). The first sub-mechanism 750 may further include a proportional valve 753 configured to control a flow of the second gas introduced from the first gas source 741 (via the first-second gas entrance 7021). In some embodiments, the first sub-mechanism 750 may further include a pressure sensor 751 configured to detect a pressure of the second gas introduced from the first gas source 741 before the second gas flows into the pressure relief valve. The first sub-mechanism 750 may further include a flow sensor 754 configured to detect the flow of the second gas flowing into the gas mixing assembly 730. In some embodiments, the second sub-mechanism 780 may include a one-way valve 781 configured to control the second gas introduced from the second gas source 742 (via the second-second gas entrance 7022) to flow into the gas mixing assembly 730. In some embodiments, the second sub-mechanism 780 may share the flow sensor 754 with the first sub-mechanism 750. That is, the flow sensor 754 may be used to detect the flow of the second gas (received from the first gas source 741 via the first-second gas entrance 7021, and/or received from the second gas source 742 via the second-second gas entrance 7022) flowing into the gas mixing assembly 730. In some embodiments, as shown in FIGS. 7C and 7D, the first-second gas entrance 7021 and the second-second gas entrance 7022 may be in fluid communication with the gas importing mechanism 710. Alternatively, in some embodiments, the first-second gas entrance 7021 and/or the second-second gas entrance 7022 may be disposed on the gas importing mechanism 710, which is not shown in FIGS. 7A-7D. An exemplary one-way valve may be shown in FIGS. 9A-9O.

As shown in FIG. 7E, the first gas entering the first gas entrance 701 through the gas filter unit 761 may flow into the gas pressurization unit 720 and be pressurized by the gas pressurization unit 720. The pressurized first gas and the second gas may be mixed by the gas mixing assembly 730. The gas passage(s) between the first gas entrance 701, the gas pressurization unit 720, and the gas mixing assembly 730 may be equipped with or coupled to a temperature and humidity sensor 762, a pressure sensor 763, and a flow sensor 764. The temperature and humidity sensor 762 may be configured to detect the temperature and humidity of the first gas. The pressure sensor 763 may be configured to detect (and/or control) the pressure of the first gas. The flow sensor 764 may be configured to detect (and/or control) a flow of the first gas. The gas passage(s) between the gas mixing assembly 730 and the humidification assembly 770 may be equipped with or coupled to an oxygen concentration sensor 767, a flow and temperature sensor 768, and a pressure sensor 769. The oxygen concentration sensor 767 may be configured to detect (and/or control) an oxygen concentration of the mixed gas. The flow and temperature sensor 768 may be configured to detect (and/or control) the flow and temperature of the mixed gas. The pressure sensor 769 may be configured to detect (and/or control) the pressure of the mixed gas. The mixed gas may be humidified (and/or warmed) by the humidification assembly 770. The humidified (and/or warmed) mixed gas may be discharged to the subject 773 via the respiration tube 771 and the subject interface 772.

In some embodiments, the gas importing mechanism 710 may be further configured to switch between an automatic gas importing manner and a manual gas importing manner. The automatic gas importing manner may be functioned by the first sub-mechanism 750. The automatic gas importing manner may be configured to import the second gas from the first gas source 741 (via the first-second gas entrance 7021) automatically. The manual gas importing manner may be functioned by the second sub-mechanism 780. The manual gas importing manner may be configured to import the second gas from the second gas source 742 (via the second-second gas entrance 7022) manually. In some embodiments, the switching between the automatic gas importing manner and the manual gas importing manner may be performed by controlling an on/off state of the proportional valve 753. For example, when the proportional valve 753 is on, the gas importing mechanism 710 may be switched to the automatic gas importing manner; when the proportional valve 753 is off, the gas importing mechanism 710 may be switched to the manual gas importing manner.

The ventilatory treatment apparatus 700 may be similar to or the same as the ventilatory treatment apparatus 200. The first-second gas entrance 7021 may be similar to or the same as the first-second gas entrance 2021. The second-second gas entrance 7022 may be similar to or the same as the second-second gas entrance 2022. The gas outlet port 707 of the main body 709 of the ventilatory treatment apparatus 700 may be similar to or the same as the gas outlet port 207 of the main body 209 of the ventilatory treatment apparatus 200. The first gas entrance 701 may be similar to or the same as the first gas entrance 201. The gas filter unit 761 may be similar to or the same as the gas filter unit 203. The humidification assembly 770 may be similar to or the same as the humidification assembly 206.

According to the gas importing mechanism shown in FIGS. 7A-7E, the gas importing mechanism can be switched between the automatic gas importing manner and the manual gas importing manner, which increases the flexibility of using the first gas source and/or the second gas source, and expands the application scenario of the ventilatory treatment apparatus 700. The first sub-mechanism 750 and the second sub-mechanism 780 can be integrated into the gas importing mechanism, and can function properly and be switched through controlling the on/off state of the proportional valve 753, without using additional elements, components, or devices, which saves cost, reduces the weight and volume of the ventilatory treatment apparatus 700.

FIGS. 8A-8C illustrate an exemplary gas intake connector assembly according to some embodiments of the present disclosure. FIG. 8A shows an axonometric drawing of the gas intake connector assembly 800. FIG. 8B shows an exploded view of the gas intake connector assembly 800. FIG. 8C shows a section view of the gas intake connector assembly 800.

The gas intake connector assembly 800 may be configured to introduce the second gas from at least one gas source (e.g., the first gas source 741 or the second gas source 742 shown in FIG. 7E), via the at least one gas entrance (e.g., the first-second gas entrance 2021 or the first-second gas entrance 7021, the second-second gas entrance 2022 or the second-second gas entrance 7022), into the ventilatory treatment apparatus 200 (or 700). The gas intake connector assembly 800 may be disposed at the at least one gas entrance. The gas intake connector assembly 800 may be connected to the at least one gas source directly or via a tube. The gas intake connector assembly 800 may include a female connector 810 and a male connector 820 operably coupled to the female connector 810. The female connector 810 may include a first pipe structure. The male connector 820 may include a second pipe structure. In some embodiments, at least a portion of the first pipe structure may include an internal thread, and at least a portion of the second pipe structure may include an external thread. Alternatively, in some embodiments, at least a portion of the first pipe structure may include an external thread, and at least a portion of the second pipe structure may include an internal thread.

In some embodiments, the gas intake connector assembly 800 may further include a stop mechanism 830 configured to prevent the male connector 820 from being separated with the female connector 810. In some embodiments, the stop mechanism 830 may include a stop screw 831 and a stop groove 832. The stop groove 832 may be disposed on the male connector 820. The stop screw 831 may be capable of entering, via a threaded hole 833 on the female connector 810, the stop groove 832. The stop screw 831 may be limited in the stop groove 832, so that the male connector 820 is not easily separated with the female connector 810.

In some embodiments, a gas filter unit may be mounted in the gas intake connector assembly 800. The gas filter unit may be configured to filter the second gas entering the ventilatory treatment apparatus 200 (or 700). In some embodiments, the gas filter unit may be disposed between the female connector 810 and the male connector 820.

FIGS. 9A-9R illustrate an exemplary one-way valve according to some embodiments of the present disclosure. FIG. 9A shows an overall view of the one-way valve 910. FIG. 9B shows a section view of the one-way valve 910 when the one-way valve 910 is in a first use state. FIG. 9C shows a section view of the one-way valve 910 when the one-way valve 910 is in a second use state. FIG. 9D shows a section view of the one-way valve 910 when the one-way valve 910 is in a third use state. FIG. 9E shows a decomposition structure of another structure of the one-way valve 910. FIG. 9F shows a section view of the one-way valve 910 of FIG. 9E when the one-way valve 910 is in a first use state. FIG. 9G shows a section view of the one-way valve 910 of FIG. 9E when the one-way valve 910 is in a second use state. FIG. 9H shows a section view of the one-way valve 910 of FIG. 9E when the one-way valve 910 is in a third use state. FIG. 9I shows a decomposition structure of a pipe assembly of the one-way valve 910. FIG. 9J shows a section view of the pipe assembly of FIG. 9I in a first use state. FIG. 9K shows a section view of the pipe assembly of FIG. 9I in a second use state. FIG. 9L shows a section view of the pipe assembly of FIG. 9I in a third use state. FIG. 9M shows a section view of the one-way valve 910 of FIG. 9F in a first use state. FIG. 9N shows a section view of the one-way valve 910 of FIG. 9F in a second use state. FIG. 9O shows a section view of the one-way valve 910 of FIG. 9F in a third use state. FIGS. 9P-9R show exemplary asymmetric hinge segments.

In some embodiments, in the use of a ventilatory treatment apparatus (e.g., the ventilatory treatment apparatus 200), a pressure of the exhaled gas of the subject 180 may be higher than the pressure of the respiratory gas provided by the apparatus, and thus, a backflow gas may be generated in the gas passages(s) inside the apparatus and/or between the apparatus and the subject 180. In some embodiments, in order to prevent the humidified gas generated in the humidification assembly 206 from flowing back into the main body 209 of the ventilatory treatment apparatus 200, a one-way valve may be disposed between the humidification assembly 206 and the main body 209 of the ventilatory treatment apparatus 200 (e.g., in the gas passage(s) inside the main body 209 and/or in the gas passage(s) between the humidification assembly 206 and the main body 209).

As shown in FIG. 9A, the one-way valve 910 may include a valve body 912 and a reverse valve body 914. When the one-way valve 910 is not in use, the reverse valve body 914 may be disposed at a first or second side of the valve body 912. When the one-way valve 910 is in use, the reverse valve body 914 may be pushed to the opening 9120, and then the one-way valve 910 may be mounted in a corresponding gas passage. The valve body 912 may include a main hinge part 9122 disposed at an edge part of the valve body 912. The valve body 912 may be configured to rotate around the main hinge part 9122 to switch the valve body 912 between an open state and a closed state. The valve body 912 may rotate from a position shown in FIG. 9B to the second side (opposite to the first side) of the valve body 912 (i.e., to the left side shown in FIG. 9B), to reach an open state (as shown in FIG. 9C). When the valve body 912 is in the open state, the forward respiratory gas in the first side of the valve body 912 (i.e., the right side shown in FIG. 9C) may pass through the valve body 912. When the forward respiratory gas in the first side of the valve body 912 flows towards the valve body 912, the valve body 912 may rotate towards the second side of the valve body 912, and the forward respiratory gas may pass through the valve body 912. When the forward respiratory gas stops flowing, the valve body 912 may rotate back to reach the closed state as shown in FIG. 9B. When the valve body 912 is in the closed state, the backward respiratory gas may be blocked by the valve body 912 and may not pass through the valve body 912 to flow back to the first side. The valve body 912 may include an opening 9120. The reverse valve body 914 may be disposed at the opening 9120. When the valve body 912 is in the closed state, the reverse valve body 914 may be opened under the propulsive force of the backward respiratory gas, to allow the backward respiratory gas to pass through the valve body 912 to reach the first side. At this time, the reverse valve body 914 is in a reverse open state. The reverse valve body 914 may be configured to rotate from the closed state (the opening 9120 is closed) shown in FIG. 9B, to the first side of the valve body 912 (i.e., the right side shown in FIG. 9B), to reach the reverse open state shown in FIG. 9D. When the forward respiratory gas flows from the first side to the second side of the valve body 912, the valve body 912 may be opened, and the reverse valve body 914 may be closed to close the opening 9120. When the backward respiratory gas flows from the second side to the first side of the valve body 912, the reverse valve body 914 may be opened to reach the reverse open state, and the valve body 912 may be closed. Therefore, the first one-way open of the valve body 912 may be guaranteed to allow the forward respiratory gas to pass through, and the second one-way open (opposite to the first one-way open) of the reverse valve body 914 may be allowed, and thus, the forward respiratory gas and the backward respiratory gas may have their respective paths.

According to FIGS. 9I, 9J and 9M, the one-way valve 910 may be used in a high flow ventilatory treatment apparatus. A main body of the high flow ventilatory treatment apparatus may be in the first side of the valve body 912, the subject 180 may be in the second side of the valve body 912. In the high flow working mode of the apparatus, the main body may provide respiratory gas to the subject 180. As shown in FIGS. 9K and 9N, the forward respiratory gas may push the valve body 912 of the one-way valve 910, and pass through the one-way valve 910 to reach the subject 180, while the reverse valve body 914 may be in a closed state to close the opening 9120. In the ventilator working mode of the apparatus, when the subject is in the inspiratory state, the forward respiratory gas may push the valve body 912 of the one-way valve 910, and pass through the one-way valve 910 to reach the subject 180, while the reverse valve body 914 may be in a closed state to close the opening 9120. In the ventilator working mode of the apparatus, when the subject is in the expiratory state, as shown in FIGS. 9L and 9O, the valve body 912 may be in the closed state, the gas discharged from the subject 180 (i.e., the backward respiratory gas) may open the reverse valve body 914 and may pass through the reverse valve body 914, thereby releasing the pressure in the humidification assembly and the gas passage(s), and alleviating suffocation when the subject 180 exhales. When the apparatus is not in use, as shown in FIGS. 9J and 9M, the valve body 912 and the reverse valve body 914 may be both in the closed state, thereby preventing the humidified gas in the humidification assembly to flow back into the main body of the apparatus.

In some embodiments, the one-way valve 910 may be configured as an integral piece. In some embodiments, in order to improve the airtightness of the one-way valve 910 in the gas passage(s), the one-way valve 910 may be configured as an clastic piece.

In some embodiments, the reverse valve body 914 may be mounted on the valve body 912 through a reverse hinge part. The reverse valve body 914 may rotate around the reverse hinge part, to realize the opening and the closing of the reverse valve body 914. The reverse hinge part may be configured as an clastic piece. For example, the reverse hinge part may be made of an elastic material (e.g., silica gel). When the reverse valve body 914 is pushed by the backward respiratory gas, the clastic piece (of the reverse hinge part) may be stretched; when the propulsive force is released, the clastic piece may be retracted to drive the reverse valve body 914 to close the opening 9120. According to FIGS. 9J, 9K, 9M and 9N, in an initial state, or when the apparatus is not in use, or when the forward respiratory gas passes through the one-way valve 910, the reverse hinge part may apply tension on the reverse valve body 914 to make the reverse valve body 914 close the opening 9120. According to FIGS. 9L and 9O, when the backward respiratory gas pushes the reverse valve body 914, the reverse hinge part may be stretched, and the reverse valve body 914 may be opened to reach the reverse open state. When the backward respiratory gas stops flowing (i.e., the propulsive force of the backward respiratory gas is released), the reverse hinge part may be retracted and drive the reverse valve body 914 to move back to close the opening 9120.

According to FIGS. 9B-9D, the reverse hinge part may include a hinge segment 9140. Two ends of the hinge segment 9140 may be connected to the valve body 912 and the reverse valve body 914, respectively. The hinge segment 9140 may be configured as an elastic piece. With the use of the hinge segment 9140, the reverse valve body 914 may be opened or closed. In some embodiments, hinge segment 9140, the valve body 912, and the reverse valve body 914 may be configured as an integral piece. According to FIGS. 9P-9R, the reverse hinge part may include one or more hinge segments 9140 (e.g., one hinge segment 9140 shown in FIG. 9Q, two hinge segments 9140 shown in FIG. 9P, three hinge segments 9140 shown in FIG. 9R). The hinge segment(s) 9140 may be asymmetrically distributed along a central axis plane (along a radial direction of the valve body 912) of the insertion part 9160. Therefore, when the reverse valve body 914 is opened, the left side and the right side of the reverse valve body 914 may have different resonant frequencies, that is, the reverse valve body 914 may have no constant resonant frequency, thereby reducing or eliminating resonance of the reverse valve body 914, and reducing or avoiding abnormal sound generated by the resonance of the reverse valve body 914.

According to FIGS. 9E-9F, the reverse hinge part may include a convex lug 9142 mounted on the valve body 912. The convex lug 9142 may be mounted on the valve body 912 through a fastening piece 9144. The convex lug 9142 may be configured as an elastic piece. With the use of the convex lug 9142, the reverse valve body 914 may be opened or closed. According to FIGS. 9F-9G, when the forward respiratory gas flows from the first side to the second side of the valve body 912, the forward respiratory gas may push the valve body 912 to make the valve body 912 open. As shown in FIG. 9G, the valve body 912 is in the open state. The valve body 912 may rotate from the position shown in FIG. 9F to the second side (i.e., the left side shown in FIG. 9F), to allow the forward respiratory gas to pass through. As shown in FIGS. 9F and 9H, the valve body 912 is in the closed state. When the backward respiratory gas flows from the second side to the first side, the backward respiratory gas may push the reverse valve body 914 to make the reverse valve body 914 open. The reverse valve body 914 may rotate from the position shown in FIG. 9F to the first side (i.e., the right side shown in FIG. 9F). As shown in FIG. 9I, the reverse valve body 914 is in the reverse open state to allow the backward respiratory gas to flow back.

According to FIGS. 9B, 9D and 9I, the valve body 912 may be equipped with a stopping part 9124. The stopping part 9124 may protrude towards a center of the opening 9120. When the reverse valve body 914 rotates towards the second side, the stopping part 9124 may abut the reverse valve body 914 to prevent the reverse valve body 914 from crossing the valve body 912 and reaching the second side. With the use of the stopping part 9124, the reverse valve body 914 may not cross the valve body 912 to reach the second side, and thus, the reverse valve body 914 may only rotate to the first side to allow the backward respiratory gas to pass through, thereby guaranteeing the airtightness of the one-way valve 910. In some embodiments, an inner diameter of the opening 9120 may be no larger than (e.g., smaller than) an outer diameter of the reverse valve body 914, and thus, the contact part of the valve body 912 and the reverse valve body 914 may form the stopping part 9124 (i.e., the edge portion of the opening 9120 may form the stopping part 9124).

According to FIGS. 9A-9B, the valve body 912 may be equipped with a first protruding reinforced rib 9126. The first protruding reinforced rib 9126 may extend along a thickness direction of the valve body 912. The first protruding reinforced rib 9126 may be configured to increase the strength of the valve body 912. The first protruding reinforced rib 9126 may be disposed at the second side of the valve body 912. In some embodiments, the first protruding reinforced rib 9126 may be of a ring shape.

In some embodiments, the reverse valve body 914 may be equipped with a second protruding reinforced rib 9146. The second protruding reinforced rib 9146 may extend along a thickness direction of the reverse valve body 914. The second protruding reinforced rib 9146 may be configured to increase the strength of the reverse valve body 914. The second protruding reinforced rib 9146 may be disposed at the second side of the reverse valve body 914 (i.e., the second side of the valve body 912). In some embodiments, the second protruding reinforced rib 9146 may be of a ring shape.

In some embodiments, the one-way valve 910 may be equipped with an assembly part 916. The assembly part 916 may include an insertion part 9160. The insertion part 9160 may facilitate the valve body 912 to be inserted into a tube 922 that forms a segment of the gas passage(s). The main hinge part 9122 may be connected to the insertion part 9160. The insertion part 9160 may be elastic, so as to facilitate the valve body 912 to be inserted into the tube 922 conveniently and be self-sealing. The main hinge part 9122 and the insertion part 9160 may be configured as an integral piece.

In some embodiments, the insertion part 9160 may be equipped with a first stopping lug boss 9184 configured to stop the insertion part 9160 from moving in the tube 922. The first stopping lug boss 9184 may be positioned outside the tube 922 when the valve body 912 is inserted into the tube 922. The first stopping lug boss 9184 may be of a ring shape. The first stopping lug boss 9184 may seal the connection between the insertion part 9160 and the tube 922, thereby guaranteeing the airtightness of the tube 922.

In some embodiments, the insertion part 9160 may be equipped with a second stopping lug boss 9186 configured to stop the valve body 912 from moving out the tube 922. The second stopping lug boss 9186 and the first stopping lug boss 9184 may be positioned at a proper interval. The second stopping lug boss 9186 may seal the connection between the insertion part 9160 and the tube 922, thereby guaranteeing the airtightness of the tube 922.

In some embodiments, the tube 922 and the one-way valve 910 disposed in the tube 922 may form a tube assembly 920. The one-way valve 910 may be used in the ventilatory treatment apparatus 200. The forward respiratory gas and backward respiratory gas in the tube 922 may pass through the one-way valve 910, and the forward respiratory gas and the backward respiratory gas may have their respective paths.

In some embodiments, the tube assembly 920 may be equipped with an assembly port 9220. The one-way valve 910 may be equipped with an assembly part 916. The assembly part 916 may include an insertion part 9160. The insertion part 9160 may facilitate the valve body 912 to be inserted into the assembly port 9220. The main hinge part 9122 may be connected to the insertion part 9160. In some embodiments, a limiting structure may be disposed between the insertion part 9160 and a wall around the assembly port 9220. The limiting structure may be configured to make the insertion part 9160 be firmly mounted on the assembly port 9220. The limiting structure may include a limiting lug boss 9180 and a limiting groove 9182. The limiting groove 9182 may be configured to accommodate the limiting lug boss 9180 when the limiting lug boss 9180 inserts the limiting groove 9182. The limiting lug boss 9180 may be disposed on the insertion part 9160, and the limiting groove 9182 may be disposed in the wall of the assembly port 9220. Alternatively, the limiting lug boss 9180 may be disposed in the wall of the assembly port 9220, and the limiting groove 9182 may be disposed on the insertion part 9160. The insertion part 9160 and the assembly port 9220 may be firmly connected through the cooperation of the limiting lug boss 9180 and the limiting groove 9182, which guarantees the airtightness of the tube 922.

In some embodiments, a sealing lug boss 9240 may be disposed in the inner wall of the tube assembly 920. The sealing lug boss 9240 may be equipped with an inclined sealing face 9242. The inclined sealing face 9242 may abut the valve body 912. The inclined sealing face 9242 may be gradually close to the valve body 912 from the outside to the inside of the assembly port 9220. Therefore, after the one-way valve 910 is mounted in the tube assembly 920, the inclined sealing face 9242 may abut the valve body 912 closely, thereby guaranteeing the airtightness of the tube assembly 920. In some embodiments, the assembly port 9220 may be disposed at the top of the tube assembly 920, and the inclined sealing face 9242 may be gradually close to the valve body 912 under the gravity direction. Therefore, after the one-way valve 910 is mounted in the tube assembly 920, the inclined sealing face 9242 may abut the valve body 912 closely under the force of gravity.

In some embodiments, a stopping lug boss 9244 may be disposed on the inner wall of the tube assembly 920 to guarantee the one-way open of the valve body 912. The stopping lug boss 9244 may be configured to abut the valve body 912 to stop the valve body 912. Therefore, when the backward respiratory gas passes through the valve body 912, the valve body 912 may be closed, thereby reducing the humidified gas flowing back to the main body when the apparatus is not in use.

With the use of the tube assembly 920, the forward respiratory gas and the backward respiratory gas may have their respective paths, thereby guaranteeing the normal performance of the apparatus in the high flow working mode, and the ventilator working mode. In addition, the humidified gas may be prevented from flowing back to the main body, thereby protecting the apparatus. The gas discharged from the subject 180 (i.e., the backward respiratory gas) may open the reverse valve body 914 and may pass through the reverse valve body 914, thereby alleviating suffocation when the subject 180 exhales.

FIGS. 10 and 11 illustrate an exemplary ventilatory treatment apparatus according to some embodiments of the present disclosure. FIG. 10 shows an overall view of the ventilatory treatment apparatus 1000. FIG. 11 shows a section view of the ventilatory treatment apparatus 1000.

In some embodiments, as shown in FIG. 10 and FIG. 11, a ventilatory treatment apparatus 1000 may include a main body 1010 and a humidification assembly 1020 that is mounted on the main body 1010. The main body 1010 may include a mounting base used for an installation of the humidification assembly 1020. The humidification assembly 1020 may be separated from the mounting base, so as to add liquid in the humidification assembly 1020, clean the humidification assembly 1020, etc.

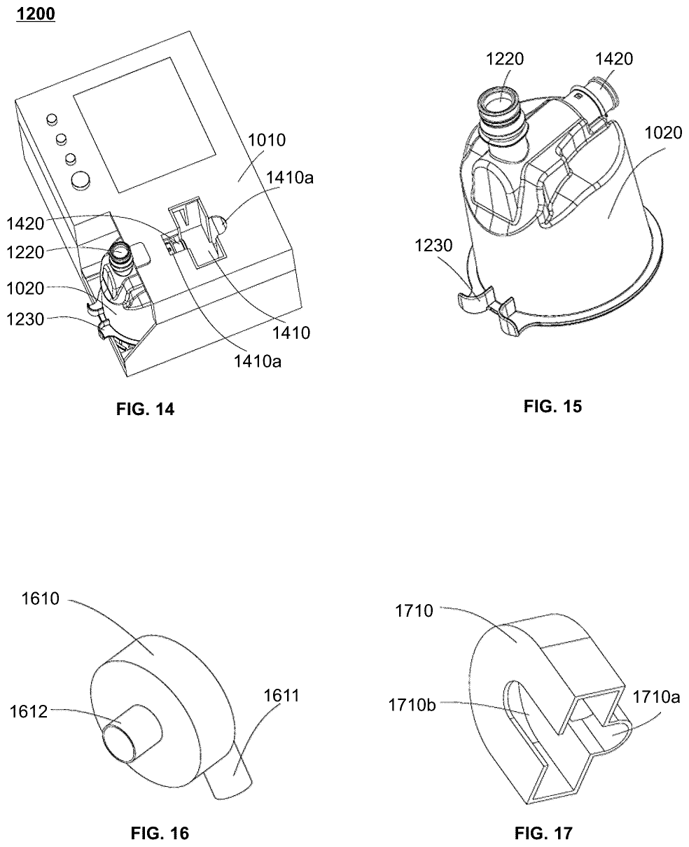

FIGS. 12-14 illustrate overall views of an exemplary ventilatory treatment apparatus according to some embodiments of the present disclosure. FIG. 12 shows an overall view of the ventilatory treatment apparatus 1200. FIG. 13 shows an overall view of the ventilatory treatment apparatus 1200 without the humidification assembly. FIG. 14 shows an overall view of the ventilatory treatment apparatus 1200 without the gas filter unit.

In some embodiments, as shown in FIGS. 12-14, the main body 1010 may include a mounting slot 1410 for a gas filter unit, so as to provide a filtered and humidified gas to the subject. The main body 1010 may further include a gas outlet port facing the mounting slot 1410 for the gas filter unit. The mounting slot 1410 for the gas filter unit may be used for an installation of a gas filter unit 1030, a ventilatory tube (not shown in the figures), etc. A gas inlet port of the gas filter unit 1030 or the ventilatory tube may be connected to the gas outlet port of the main body 1010. A gas outlet port of the gas filter unit 1030 or the ventilatory tube may be in communication with a liquid chamber of the humidification assembly 1020. The gas outlet port of the main body 1010 may be in communication with the liquid chamber of the humidification assembly 1020 through the gas filter unit 1030 or the ventilatory tube. A respiration gas that is filtered or not filtered may be provided to the subject through an assembly that is mounted on the mounting slot 1410.

In some embodiments, the gas filter unit 1030 may be disposed in or removed from the gas path of the ventilatory treatment apparatus 1000. That is, a complete gas path may also be formed without the gas filter unit 1030, thereby satisfying actual requirements of the subject. For example, the gas filter unit 1030 may be mounted in the mounting slot 1410 to filter the gas flowing from the gas outlet port of the main body 1010, and the filtered gas may be humidified in the humidification assembly 1020 to be provided to the subject. As another example, the gas filter unit 1030 mounted in the mounting slot 1410 may be replaced by the ventilatory tube that is disposed between the gas outlet port of the main body 1010 and the humidification assembly 1020, so as to ensure a completeness of the gas path. That is, the gas flowing from the gas outlet port of the main body 1010 may flow into the humidification assembly 1020 and may be humidified in the humidification assembly 1020, thereby satisfying users with different requirements and ensuring a normal usage of the ventilatory treatment apparatus 1000.

FIG. 15 illustrates an exemplary humidification assembly according to some embodiments of the present disclosure.

In some embodiments, the humidification assembly 1020 may be replaced by another existing humidification assembly with a same standard of the humidification assembly 1020. A gas inlet port of the humidification assembly 1020 may be connected to the main body 1010 as shown in FIGS. 10-14.

In some embodiments, as shown in FIG. 15, the humidification assembly 1020 may include a liquid chamber accommodated by a tank body of the humidification assembly 1020, and a gas intake tube 1420 and a gas exit tube 1220 that are in communication with the liquid chamber. The gas intake tube 1420 and the gas exit tube 1220 may be disposed on a connecting tube, and the gas exit tube 1220 may be connected with a respiration tube 1040 (shown in FIG. 10 and FIG. 11) that is configured to provide the gas to the subject. The gas outlet port of the gas filter unit 1030 or the ventilatory tube may be in communication with the liquid chamber of the humidification assembly 1020. That is, the gas outlet port of the gas filter unit 1030 or the ventilatory tube may be in communication with the liquid chamber of the humidification assembly 1020 through the gas intake tube 1420.

In some embodiments, the mounting slot 1410 may be disposed on a top of the main body 1010. That is, an opening of the mounting slot 1410 may face a top sidewall of the main body 1010. The gas filter unit 1030 may be mounted in the mounting slot 1410 from top to bottom, thereby facilitating a detachment of the gas filter unit 1030, avoiding inadequate sealing of the gas path caused by accidental impact on the gas filter unit 1030, etc.

FIG. 16 illustrates an exemplary gas filter unit according to some embodiments of the present disclosure. FIG. 17 illustrates an exemplary filter box of a gas filter unit according to some embodiments of the present disclosure.

In some embodiments, as shown in FIG. 16 and FIG. 17, the gas filter unit 1030 may include a filter body 1610 and a filter box 1710 covering the filter body 1610. The filter body 1610 may include a filter element to filter bacteria or viruses in the gas flowing from the gas outlet port of the main body 1010. The filter box 1710 may cover the filter body 1610 to define a mounting position of the gas filter unit 1030 in the mounting slot 1410. An outer contour of the filter body 1610 may be matched with an inner contour of the filter box 1710, so as to restrict a position of the filter body 1610, thereby ensuring a stable connection between the gas outlet port of the main body 1010 and the humidification assembly 1020. When the gas filter unit 1030 is mounted in the mounting slot 1410, at least part of the outer surface of the filter box 1070 may be attached to an inner sidewall of the mounting slot 1410, thereby ensuring a stable installation of the filter box 1710 in the mounting slot 1410. As shown in FIGS. 10-13, the gas filter unit 1030 may be inserted and mounted in the mounting slot 1410 from top to bottom, and a front side and a rear side (e.g., a left side and a right side of the gas filter unit 1030 shown in FIG. 11) of the filter box 1710 may be attached to sidewalls of the mounting slot 1410, respectively, thereby locating the gas filter unit 1030 in a forward direction and a backward direction, respectively. When the gas filter unit 1030 reaches to a bottom of the mounting slot 1410, a bottom surface of the filter box 1710 may be supported by a bottom surface of the mounting slot 1410, thereby locating the gas filter unit 1030 in a vertical direction. The filter body 1610 (i.e., a gas intake tube 1611 of the gas filter unit 1030 shown in FIG. 16) may be connected to the gas outlet port of the main body 1010, and the filter body 1610 (i.e., a gas exit tube 1612 of the gas filter unit 1030 shown in FIG. 16) may correspond to the gas intake tube 1420 of the humidification assembly 1020.

In some embodiments, the filter body 1610 may include the gas intake tube 1611 configured to be connected to the gas outlet port of the main body 1010, and the gas exit tube 1612 configured to be connected to the humidification assembly 1020. In some embodiments, the filter box 1710 may include a concave chamber 1710a configured to accommodate at least part of the gas intake tube 1611 and/or a groove 1710b. The gas exit tube 1612 may penetrate the groove 1710b. A part of the filter body 1610 configured to accommodate the gas filter unit 1030 may be in a regular shape, such as in a shape of circular plate, etc. The gas intake tube 1611 and the gas exit tube 1612 that are convex relative to the part of the filter body 1610 configured to accommodate the gas filter unit 1030 may be connected to the gas outlet port of the main body 1010 and the humidification assembly 1020, respectively, thereby facilitating the manufacturing of the filter body 1610 and ensuring a stable location and/or connection.

In some embodiments, as shown in FIG. 11 and FIG. 16, when the gas filter unit 1030 is mounted in the mounting slot 1410, the gas intake tube 1611 may extend downward along a vertical direction on a side of the filter body 1610, and the gas exit tube 1612 may extend along a horizontal direction on the other side of the filter body 1610, thereby extending a length of a gas flow path when the gas flows through the gas filter unit 1030 and ensuring a filter performance. Since the gas intake tube 1611 and the gas exit tube 1612 are convex relative to the filter body 1610, as shown in FIG. 14, the mounting slot 1410 may further include a corresponding concave part 1410a, so that when the gas filter unit 1030 is inserted and mounted in the mounting slot 1410 from top to bottom, the gas intake tube 1611 and the gas exit tube 1612 may reach to a mounting position through the concave part 1410a. The gas filter unit 1030 may be separated from the main body 1010 and replaced by the ventilatory tube through the concave part 1410a.

In some embodiments, the humidification assembly 1020 may be mounted on the main body 1010 along a horizontal direction, so that the gas intake tube 1420 of the humidification assembly 1020 may be connected to the gas exit tube 1612 of the filter body 1610 extending along the horizontal direction. The main body 1010 may further include a guiding rail 1210 extending along the horizontal direction, so that the humidification assembly 1020 may be mounted on the main body 1010 along the guiding rail 1210, and the gas intake tube 1420 of the humidification assembly 1020 may be connected to the gas exit tube 1612 of the filter body 1610. For instance, the gas filter unit 1030 may be mounted in the mounting slot 1410 from top to bottom, the gas intake tube 1611 of the filter body 1610 may be connected to the gas outlet port of the main body 1010, the humidification assembly 1020 may be pushed along the guiding rail 1210 to guide the humidification assembly 1020 to reach to a mounting position, and the gas intake tube 1420 of the humidification assembly 1020 may be connected to the gas exit tube 1612 of the gas filter unit 1030 in a sealed manner, thereby facilitating an assembly operation.

In some embodiments, the humidification assembly 1020 may include a handle 1230. The handle 1230 may be disposed on an outer sidewall of the humidification assembly 1020, so as to facilitate a detachment and/or an installation of the humidification assembly 1020 through a push-and-pull manner. When the gas intake tube 1420 of the humidification assembly 1020 and the gas exit tube 1612 of the gas filter unit 1030 are in a strong connection, the handle 1230 may reduce a difficulty of pulling the humidification assembly 1020 from the main body 1010.

A sealed connection between the gas filter unit 1030 and the gas outlet port of the main body 1010, or a sealed connection between the gas filter unit 1030 and the gas intake tube 1420 of the humidification assembly 1020 may be ensured to satisfy actual requirements of different subjects by selecting the gas filter unit 1030 and the ventilatory tube. In some embodiments, an end of the gas intake tube 1611 may include an inner cone surface, and the gas outlet port of the main body 1010 may include an outer cone surface that is matched with the inner cone surface. In some embodiments, an end of the gas intake tube 1611 may include an outer cone surface, and the gas outlet port of the main body 1010 may include an inner cone surface that is matched with the outer cone surface. In some embodiments, an end of the gas exit tube 1612 may include an inner cone surface, and an end of the gas intake tube 1420 of the humidification assembly 1020 may include an outer cone surface that is matched with the inner cone surface. In some embodiments, an end of the gas exit tube 1612 may include an outer cone surface, and the gas intake tube 1420 of the humidification assembly 1020 may include an inner cone surface that is matched with the outer cone surface. A sealing performance of the gas path may be ensured based on the arrangement of the inner cone surface and the outer cone surface in an assembly state. In some embodiments, the inner cone surface and the outer cone surface may be standard cone surfaces with a diameter of 22 mm. In some embodiments, the inner cone surface and the outer cone surface may be in a sealed connection based on a sealing ring along a radial direction, thereby further ensuring the sealing performance of the connection position.

In some embodiments, when the gas filter unit 1030 is not required based on the actual requirements of the subject, the gas filter unit 1030 may be replaced by the ventilatory tube. In some embodiments, the ventilatory tube may be in a shape that is the same as a shape of the gas filter unit 1030, and an inside of the ventilatory tube may not be provided with a filter element, such as a filter cotton, etc. The actual requirements of different subjects may be satisfied without replacing the ventilatory treatment apparatus 1000, thereby reducing costs of purchasing devices and saving resources.

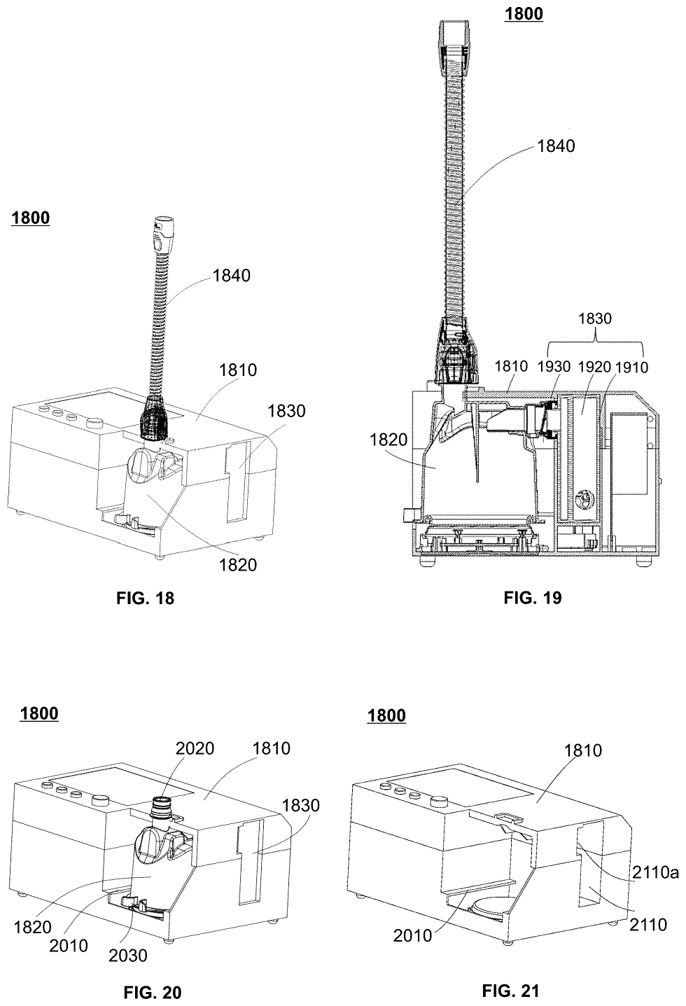

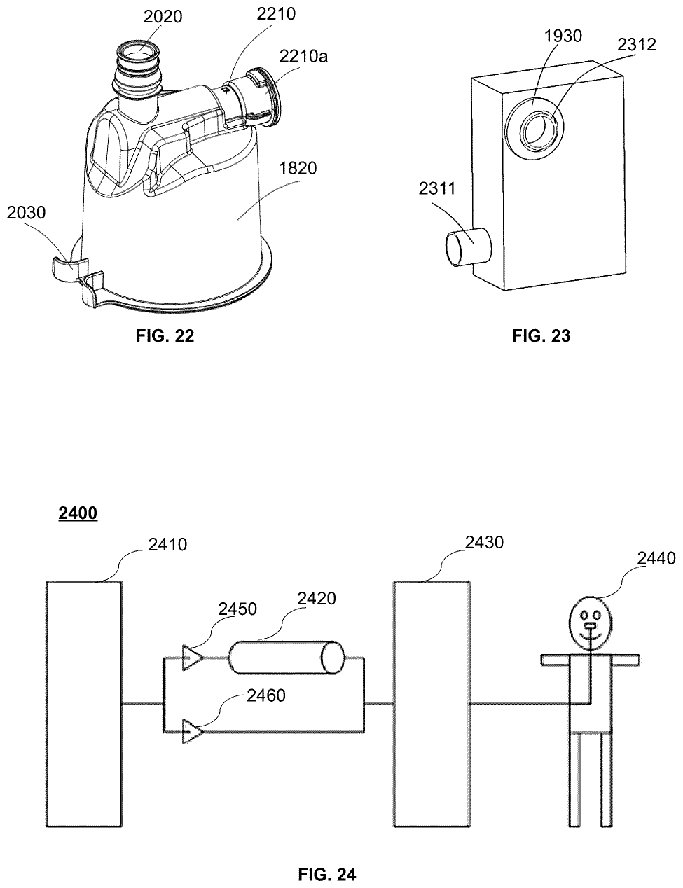

FIGS. 18-21 illustrate an exemplary ventilatory treatment apparatus according to some embodiments of the present disclosure. FIG. 22 illustrates an exemplary humidification assembly according to some embodiments of the present disclosure. FIG. 23 illustrates an exemplary gas filter unit according to some embodiments of the present disclosure.

In some embodiments, as shown in FIG. 18 and FIG. 19, the ventilatory treatment apparatus 1800 may include a main body 1810 and a humidification assembly 1820 that is mounted on the main body 1810. The main body 1810 may include a mounting base used for an installation of the humidification assembly 1820. The humidification assembly 1820 may be separated from the mounting base, so as to add liquid in the humidification assembly 1820, clean the humidification assembly 1820, etc.

In some embodiments, as shown in FIG. 20 and FIG. 21, the main body 1810 may include a mounting slot 2110 for a gas filter unit, so as to provide a filtered and humidified gas to the subject. The main body 1810 may further include a gas outlet port facing the mounting slot 2110. A gas filter unit 1830 may be inserted into and/or separated from the mounting slot 2110. A gas inlet port of the gas filter unit 1830 may be connected to a gas outlet port of the main body 1810, and a gas outlet port of the gas filter unit 1830 may be in communication with a liquid chamber of the humidification assembly 1820, so that the gas outlet port of the main body 1810 may be in communication with the liquid chamber of the humidification assembly 1820 through the gas filter unit 1830.

In some embodiments, a respiration gas provided to a subject may be filtered by the gas filter unit 1830 that is inserted and/or mounted in the mounting slot 2110, so as to satisfy actual requirements of the subject that is sensitive to bacteria or viruses in an ambient atmosphere. In some embodiments, the gas filter unit 1830 may be separated from the mounting slot 2110 and replaced by a ventilatory tube to ensure a completeness of the gas path. The ventilatory tube may be in a structure that is similar to a structure of the gas filter unit 1830, and the ventilatory tube may not be provided with a filter element. The gas flowing from the gas outlet port of the main body 1810 may flow into the humidification assembly 1820 directly and may be humidified in the humidification assembly 1820. The ventilatory treatment apparatus 1800 may satisfy actual requirements of different subjects and may be in a normal working state under different conditions. In some embodiments, a structure of the mounting slot 2110 may be matched with a structure of the gas filter unit 1830, so that the respiration gas may be filtered through a non-standard gas filter unit. In some embodiments, the mounting slot 2110 may be disposed on a sidewall of the main body 1810, thereby avoiding an increase in a height of the ventilatory treatment apparatus 1800 caused by an installation of the gas filter unit 1830 and ensuring the stability of the ventilatory treatment apparatus 1800.

In some embodiments, as shown in FIGS. 19-23, the gas filter unit 1830 may be in a non-standard structure, such as in a shape that is substantially the same as a rectangle. In some embodiments, the gas filter unit 1830 may include a filter box 1910 and a filter element 1920 disposed in the filter box 1910. The filter element 1920 may be of any material and a structure that are capable of filtering the bacteria or viruses in the air, such as a non-woven filter cotton, a glass fiber filter cotton, an activated carbon filter cotton, etc. When the gas filter unit 1830 is mounted in the mounting slot 2110, at least part of outer surface of the filter box 1910 may be attached to an inner sidewall of the mounting slot 2110.

In some embodiments, four sidewalls (e.g., a top sidewall, a bottom sidewall, a left sidewall, and a right sidewall shown in FIG. 19) of the filter box 1910 may be attached to the inner sidewall of the mounting slot 2110 along an inserting and mounting direction of the gas filter unit 1830, respectively. An inner side of the filter box 1910 along the inserting and mounting direction may abut against a bottom sidewall of the mounting slot 2110 that is opposite to an opening end of the mounting slot 2110, so that the filter box 1910 may be located in the mounting slot 2110 in a stable state, thereby avoiding a poor sealing performance of the gas path caused by a relative movement between the filter box 1910 and the ventilatory treatment apparatus 1800.

In some embodiments, the filter box 1910 may include a gas intake tube 2311 configured to be connected to the gas outlet port of the main body 1810 and a gas exit tube 2312 configured to be connected to a gas intake tube 2210 of the humidification assembly 1820 shown in FIG. 22. In some embodiments, the gas intake tube 2311 may include a gas inlet port configured to receive the gas flowing from the gas outlet port of the main body 1810. The gas exit tube 2312 may include a gas outlet port. The gas flowing from the gas outlet port of the main body 1810 may flow into the humidification assembly 1820 to be humidified by passing through the filter element 1920 and the gas outlet port of the gas exit tube 2312. In some embodiments, the gas intake tube 2311 may be disposed on the inner side of the filter box 1910 along the inserting and mounting direction. The gas intake tube 2311 may be connected to the gas outlet port of the main body 1810 in the mounting slot 2110 during an inserting and mounting operation, so that an independent connection operation may not be required. In some embodiments, the gas intake tube 2311 may be disposed close to a bottom of the filter box 1910, and the gas exit tube 2312 may be disposed close to a top of the filter box 1910, thereby extending a length of the gas path when the gas flows through the filter element 1920.

In some embodiments, the gas exit tube 2312 may be convex from a horizontal surface of the filter box 1910 along a direction that is perpendicular to the inserting and mounting direction of the gas filter unit 1830, so that the gas exit tube 2312 may be connected to the gas intake tube 2210 of the humidification assembly 1820. In some embodiments, the mounting slot 2110 may include a concave part 2110a. The concave part 2110a may allow the gas exit tube 2312 passing through the concave part 2110a when the gas filter unit 1830 is inserted and mounted in the mounting slot 2110.

In some embodiments, when the gas filter unit 1830 is mounted in the mounting slot 2110, the gas exit tube 2312 may extend toward the humidification assembly 1820 along a horizontal direction, and the gas intake tube 2210 of the humidification assembly 1820 may be connected to the gas exit tube 2312 when the humidification assembly 1820 is mounted on the main body 1810 along a horizontal direction. In some embodiments, the main body 1810 may further include a guiding rail 2010 extending along a horizontal direction, so that the humidification assembly 1820 may be mounted on the main body 1810 along the guiding rail 2010, and the gas intake tube 2210 of the humidification assembly 1820 may be connected to the gas exit tube 2312, so that a sealed gas path may be formed based on the gas filter unit 1830, thereby facilitating an assembly operation.

In some embodiments, as shown in FIG. 22, the humidification assembly 1820 may include a handle 2030. The handle 2030 may be disposed on an outer sidewall of the humidification assembly 1820, so as to facilitate a detachment and/or an installation of the humidification assembly 1820 through a push-and-pull manner. When the gas intake tube 2210 of the humidification assembly 1820 and the gas exit tube 2312 of the gas filter unit 1830 are in a strong connection, the handle 2030 may reduce a difficulty of pulling the humidification assembly 1820 from the main body 1810. For instance, the humidification assembly 1820 may include a tank body including the liquid chamber, and the gas intake tube 2210 and a gas exit tube 2020 that are in communication with the liquid chamber. The gas intake tube 2210 and the gas exit tube 2020 may be disposed on a connecting tube, and the gas exit tube 2020 of the humidification assembly 1820 may be connected to a respiration tube 1840 (shown in FIG. 18 and FIG. 19) configured to provide the gas to the subject. In some embodiments, the gas outlet port of the gas filter unit 1830 may be in communication with the liquid chamber of the humidification assembly 1820. That is, the gas outlet port of the gas filter unit 1830 may be in communication with the liquid chamber of the humidification assembly 1820 through the gas intake tube 2210 of the humidification assembly 1820.

In some embodiments, an end of the gas intake tube 2311 and an end of the gas exit tube 2312 may include a non-cone surface, respectively. In some embodiments, a connection between the gas intake tube 2311 and the gas outlet port of the main body 1810, and a connection between the gas exit tube 2312 and the gas intake tube 2210 of the humidification assembly 1820 may be sealed through a sealing ring. For instance, as shown in FIG. 19, an end of the gas exit tube 2312 may be provided with a sealing ring 1930, so that the gas exit tube 2312 may be connected to the gas intake tube 2210 of the humidification assembly 1820 in a sealed manner when the gas filter unit 1830 is inserted and mounted in the mounting slot 2110 of the gas filter unit.

In some embodiments, a free end of the gas intake tube 2210 of the humidification assembly 1820 configured to be connected to the gas exit tube 2312 may include an expanded opening part 2210a with a greater radial size relative to other parts. The gas exit tube 2312 may be inserted into and connected to the expanded opening part 2210a in a sealed manner through the sealing ring 1930.

In some embodiments, when the gas filter unit 1830 is not needed based on the actual requirements of the subject, the gas filter unit 1830 may be replaced by a ventilatory tube. In some embodiments, the ventilatory tube may be in a shape that is the same as the shape of the gas filter unit 1830, and an inside of the ventilatory tube may not be provided with a filter element, such as a filter cotton, etc. The actual requirements of different subjects may be satisfied without replacing the ventilatory treatment apparatus 1800, thereby reducing costs of purchasing devices and saving resources.

FIG. 24 illustrates an exemplary gas mixing system for ventilatory treatment according to some embodiments of the present disclosure.

As illustrated in FIG. 24, the gas mixing system 2400 for ventilatory treatment may include an oxygenerator 2410, an oxygen storage assembly 2420, a ventilatory treatment apparatus 2430. In some embodiments, the components (e.g., the oxygenerator 2410, the oxygen storage assembly 2420, or the apparatus 2430) of the gas mixing system 2400 may be in a fluid communication with each other with gas pipes.

In some embodiments, the oxygen storage assembly 2420 may suck air (e.g., a first gas) from the external space (e.g., a gas source). In some embodiments, a gas outlet port of the oxygenerator 2410 may be connected with the oxygen storage assembly 2420 (e.g., a gas inlet port of the oxygen storage assembly 2420) and the apparatus 2430 (e.g., a gas pressurization unit or a gas importing mechanism of the apparatus 2430), respectively. Thus, the oxygen generated in the oxygenerator 2410 (e.g., a second gas) may be supplied to the oxygen storage assembly 2420 and the apparatus 2430, respectively. In some embodiments, the oxygenerator 2410 or the oxygen storage assembly 2420 may be used as a gas source of the second gas (e.g., oxygen) for the apparatus 2430.

In some embodiments, the oxygen storage assembly 2420 may be integrated in the oxygenerator 2410. In some embodiments, the oxygen storage assembly 2420 may be disposed independently from the oxygenerator 2410.

In some embodiments, the apparatus 2430 may include a ventilator or a high flow humidification oxygen therapy instrument including a humidification assembly for humidifying the gas. Thus, the oxygen supplied by the oxygenerator 2410 may be humidified in the apparatus 2430.

In some embodiments, the apparatus 2430 may further include a nasal oxygen tube, and a respiratory mask connected with the apparatus 2430 with the nasal oxygen tube. Thus, the humidified oxygen may be supplied from the apparatus 2430 to an object 2440 wearing the respiratory mask. In some embodiments, the air sucked from external space may be mixed with the oxygen supplied by the oxygenerator 2410 in the apparatus 2430, and then the mixed gas may be humidified by the humidification assembly of the apparatus 2430. Finally, the mixed and humidified gas may be supplied to the object 2440 with the respiratory mask.

In some embodiments, in order to avoid wasting the oxygen generated by the oxygenerator 2410, when the object 2440 is inhaling gas, the gas outlet port of the oxygenerator 2410 may be connected with the apparatus 2430. When the object 2440 is exhaling gas, the gas outlet port of the oxygenerator 2410 may be disconnected with the apparatus 2430. As illustrated in FIG. 24, a multiple circuit may be provided between the oxygenerator 2410 and the apparatus 2430, and the oxygen storage assembly 2420 may be disposed in a branch circuit of the multiple circuit. Thus, the oxygen generated when the object 2440 is exhaling gas may be stored in the oxygen storage assembly 2420 and the gas outlet port of the oxygenerator 2410 is disconnected with the apparatus 2430. A gas outlet port of the oxygen storage assembly 2420 may be connected with the apparatus 2430. Thus, when the object 2440 is inhaling gas, the oxygen generated by the oxygenerator 2410 and/or the oxygen stored in the oxygen storage assembly 2420 may be supplied to the apparatus 2430, and supplied to the object 2440 ultimately. Thus, the object 2440 may be supplied with a great amount of oxygen that is greater than an amount of oxygen merely generated by the oxygenerator 2410, without wasting the oxygen, and the effect of the treatment may thus be improved. In some embodiments, the oxygen stored in the oxygen storage assembly 2420 may be used in other ways to avoid a waste of the oxygen, which may not be limited in the present disclosure.

In some embodiments, in order to reach a better treatment effect, the gas mixing system 2400 may further include a control assembly. As illustrated in FIG. 24, in a gas circuit between the oxygenerator 2410 and the oxygen storage assembly 2420, a check valve 2450 (i.e., the first check valve, also referred to as a one-way valve) may be provided, and a positive gas outlet port of the check valve 2450 may face the oxygen storage assembly 2420. The positive gas outlet port of the check valve 2450 may open when a gas pressure of an end of a gas inlet port of the check valve 2450 is greater than an end of the gas outlet port of the check valve 2450, and the positive gas outlet port of the check valve 2450 may close when a gas pressure of the end of the gas inlet port of the check valve 2450 is smaller than the end of the gas outlet port of the check valve 2450. The check valve 2450 may be configured to stop the oxygen (with a relatively high pressure) stored in the oxygen storage assembly 2420 flowing back to the oxygenerator 2410. Thus, the check valve 2450 improve a reliability of the gas mixing system 2400.

As described above, the gas inlet port of the oxygen storage assembly 2420 may be connected with the gas outlet port of the oxygenerator 2410, and the gas outlet port of the oxygen storage assembly 2420 may be connected with the apparatus 2430. In some embodiments, the oxygen storage assembly 2420 may be connected with the oxygenerator 2410 and the apparatus 2430 respectively with a public port, and a valve may be provided to control a connection or disconnection between the oxygen storage assembly 2420 with the oxygenerator 2410, or the apparatus 2430. For example, by using the valve, the oxygen storage assembly 2420 may be connected with the oxygenerator 2410, but disconnected with the apparatus 2430 at the same time. Thus, the storage and supplying control of the oxygen may be implemented.

As described above, the oxygen storage assembly 2420 may be disposed on the branch circuit of the multiple circuit, and thus the gas outlet port of the oxygenerator 2410 and the gas outlet port of the oxygen storage assembly 2420 may both be connected with the apparatus 2430. In order to avoid the gas flowing out from the gas outlet port of the oxygen storage assembly 2420 flowing back to the gas outlet port of the oxygenerator 2410 before flowing into the apparatus 2430, a check valve 2460 (e.g., the second valve) may be provided on a gas circuit between the gas outlet port of the oxygenerator 2410 and the apparatus 2430. The check valve 2460 may be configured to allow the oxygen to flow with a direction from the oxygenerator 2410 to the apparatus 2430, and stop the oxygen to flow back from the apparatus 2430 to the oxygenerator 2410.

As described above, when the object 2440 is exhaling gas, the gas outlet port of the oxygenerator 2410 may be disconnected with the apparatus 2430 to avoid the waste of the oxygen. Thus, a switch (not shown) may be provided on the gas circuit between the gas outlet port of the oxygenerator 2410 and the apparatus 2430 to control a connection or disconnection between the oxygenerator 2410 and the apparatus 2430. In some embodiments, the switch may be a proportional valve. In some embodiments, the switch may be an ordinary switch assembly.

As described above, different respiratory states of the object 2440 may correspond to different connection states of the oxygenerator 2410 and the apparatus 2430. For example, when the object 2440 is inhaling gas, the gas outlet port of the oxygenerator 2410 may be connected with the apparatus 2430. As another example, when the object 2440 is exhaling gas, the gas outlet port of the oxygenerator 2410 may be disconnected with the apparatus 2430. In some embodiments, a respiratory state sensor (not shown) may be disposed in the apparatus 2430, and the respiratory state sensor may be configured to detect a respiratory state of the object 2440. In some embodiments, the respiratory state sensor may be connected with the switch. Thus, when the respiratory state sensor detects that the object 2440 is inhaling gas, the switch may keep an on state, and allow the gas outlet port of the oxygenerator 2410 to be connected with the apparatus 2430. When the respiratory state sensor detects that the object 2440 is exhaling gas, the switch may turn an off state, and prevent the gas outlet port of the oxygenerator 2410 from being connected with the apparatus 2430, and then the oxygen generated by the oxygenerator 2410 may be stored in the oxygen storage assembly 2420. Next time, when the object 2440 is inhaling gas, the oxygen generated by the oxygenerator 2410 and the oxygen stored in the oxygen storage assembly 2420 may both be supplied to the object 2440 ultimately after being processed (e.g., mixed, humidified, warmed, etc.) in the apparatus 2430.

In some embodiments, the respiratory state sensor may be provided in a circuit connected with a component of the apparatus 2430 that can be worn by the object 2440. For example, the respiratory state sensor may be provided on a gas circuit connected with the respiratory mask of the apparatus 2430, or may be provided in the humidification assembly. In some embodiments, the respiratory state sensor may include a pressure sensor, a flow sensor, or a combination thereof, or the like. A wave form of an electrical signal generated by the respiratory state sensor may change with a change of the respiratory state of the object 2440, thus the on/off state of the switch may be changed based on a change of the wave form of the electrical signal.

FIGS. 25A-25B illustrate an exemplary noise reduction assembly according to some embodiments of the present disclosure. FIG. 25A shows an overall view of the noise reduction assembly 25100. FIG. 25B shows a cross-sectional view of the noise reduction assembly 25100. The noise reduction assembly 25100 may include a noise reduction box 25130, one or more separation walls 25150, and one or more connecting pipes 25140. The noise reduction box 25130 may be configured to accommodate a gas pressurization unit 25190. In some embodiments, the noise reduction box 25130 may include two or more chambers.

In some embodiments, as illustrated in FIG. 25B, the two or more chambers may include a first chamber 25160, a second chamber 25170, and a third chamber 25180. Each two adjacent chambers of the two or more chambers may be separated by one of the one or more separation walls 25150. For example, the first chamber 25160 and the second chamber 25170 may be separated by the separation wall 25151. As another example, the second chamber 25170 and the third chamber 25180 may be separated by the separation wall 25152. The separation wall(s) 25150 may have a structure of a plate (e.g., a plane plate, an inclined plate, a curved plate, etc.). In some embodiments, the separation wall(s) 25150 may have any other structure that can separate the noise reduction box into two or more chambers, which may not be limited in the present disclosure. In some embodiments, the gas pressurization unit 25190 may be disposed in one or more of the two or more chambers. In some embodiments, a gas inlet port 25110 of the first chamber 25160 and/or a gas outlet port 25120 of the gas pressurization unit 25190 may disposed on a housing of the noise reduction box 25130. In some embodiments, the housing of the noise reduction box 25130 may be divided into two portions, including a lower portion 25131 and an upper portion 25132. The gas inlet port 25110 of the first chamber 25160 may be disposed at the lower portion 25131 of the housing, and the gas outlet port 25120 of the gas pressurization unit 25190 may be disposed at the upper portion 25132 of the housing. The cross section of the noise reduction box 25130 shown in FIG. 25B may be parallel to the sidewall on which the gas inlet port 25110 and the gas outlet port 25120 are disposed.

The gas inlet port 25110 of the first chamber 25160 may be configured to introduce the respiratory gas (e.g., the first gas from the first gas entrance 201 of the ventilatory treatment apparatus 200, the mixed gas from the gas outlet port of the gas mixing assembly 730, or the like) into the first chamber 25160, thus gas from the outside of the noise reduction box may enter the first chamber 25160. The first chamber 25160 may be configured to accommodate the gas entering the first chamber 25160 via the gas inlet port 25110 of the first chamber 25160.

The gas flowing into the first chamber 25160 may flow between the two or more chambers in the noise reduction box 25130, and may be pressurized by the gas pressurization unit 25190. The gas outlet port 25120 of the gas pressurization unit 25190 may be open to the outside of the noise reduction box 25130 to discharge the pressurized gas. The gas outlet port 25120 of the gas pressurization unit 25190 may be configured to discharge the respiratory gas from the gas pressurization unit 25190 to, for example, the gas mixing assembly 730, or the gas export 204 of the ventilatory treatment apparatus 200.

The housing of the noise reduction box 25130 may be configured to accommodate components (e.g., the one or more separation walls 25150, the one or more connecting pipes 25140, or the like) of the noise reduction assembly 25100. In some embodiments, the housing 25130 may be made of plastic. Exemplary plastic may include a general plastic, an engineering plastic, or a special plastic, which may not be limited in the present disclosure.

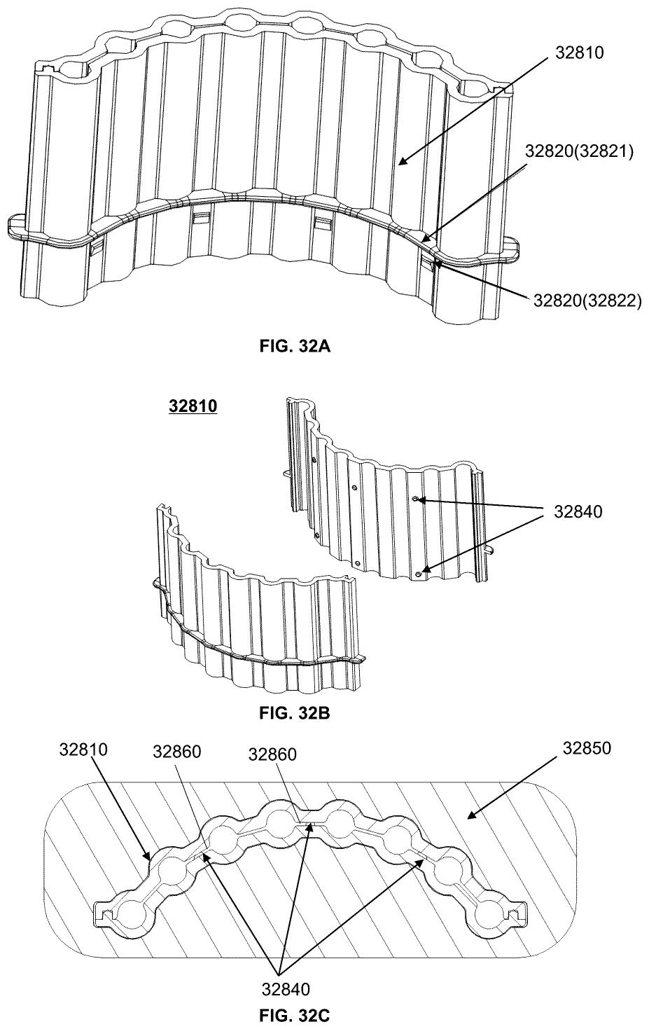

The one or more separation walls 25150 may be configured to define the two or more chambers. The one or more connecting pipes 25140 may be configured to direct the gas to flow between the two or more chambers through the one or more separation walls 25150, so that a noise generated by a flow of the gas may be reduced or eliminated. As illustrated in FIG. 25B, the one or more connecting pipes 25140 may include at least one first pipe 25141 (e.g., the connecting pipe with a relatively large length shown in FIG. 25B) and/or at least one second pipe 25142 (e.g., the connecting pipe with a relatively small length shown in FIG. 25B). The at least one first pipe 25141 and/or the at least one second pipe 25142 may pass through at least one of the one or more separation walls 25150. For example, the at least one first pipe 25141 may pass through the separation wall 25151 between the first chamber 25160 and the second chamber 25170, and the separation wall 25152 between the second chamber 25170 and the third chamber 25180, so that the first chamber 25160 is in fluid communication with the third chamber 25180 via the at least one first pipe 25141. As another example, the at least one second pipe 25142 may pass through the separation wall 25152 between the second chamber 25170 and the third chamber 25180, so that the third chamber 25180 is in fluid communication with the second chamber 25170 via the at least one second pipe 25142. In some embodiments, the one or more connecting pipes 25140 may pass through the separation wall 25150 via a flange disposed on the separation wall between the two or more chamber (or the one or more connecting pipes 25140 may be equipped with the flange). In some embodiments, the flange may include one or more through holes to let the connecting pipe(s) 25140 pass through, and the separation wall may include one or more through holes (not shown) to let the connecting pipe(s) 25140 pass through. In some embodiments, the one or more through holes may be disposed on the separation wall separately. In some embodiments, one of the one or more through holes may be in fluid communication with one or more other through holes (of the one or more through holes). In some embodiments, the one or more holes may be configured as a through hole as a whole. Exemplary connecting pipes may be shown in FIGS. 31A-32B.

The gas pressurization unit 25190 may be configured to pressurize the gas, and the pressurized gas may be discharged from the noise reduction box 25130. In some embodiments, the gas pressurization unit 25190 may be disposed between two adjacent chambers of the two or more chambers. In some embodiments, at least a portion of a shell of the gas pressurization unit 25190 may form at least a portion of a separation wall (of the one or more separation walls 25150) that separates the two adjacent chambers. For example, as illustrated in FIG. 25B, the gas pressurization unit 25190 may be disposed between the third chamber 25180 and the second chamber 25170, and at least a portion of a shell of the gas pressurization unit 25190 forms at least a portion of a separation wall that separates the third chamber 25180 and the second chamber 25170. In some embodiments, a central portion of the gas pressurization unit 25190 may be suspended in one or more of the two or more chambers (or between two adjacent chambers). In some embodiments, the shell of the gas pressurization unit 25190 may be made of soft material(s) (e.g., silica gel or soft colloid). The soft colloid may include polypropylene (PP), polyethylene (PE), or the like. Since the shell of the gas pressurization unit 25190 is made of soft material(s), vibration(s) generated during the running of the gas pressurization unit 25190 may be absorbed by the soft material(s), thus the noise may be reduced. In some embodiments, the shell of the gas pressurization unit 27360 may form a sealed connection with the separation wall 25152 and/or fix the gas pressurization unit 27360 to the separation wall 25152 and/or one or more inner walls of the noise reduction box 25130 firmly. In some embodiments, the gas pressurization unit 25190 may include one or more motors configured to pressurize the respiratory gas. In some embodiments, a count of the motor(s) may be one, and the gas pressurization unit 25190 may be disposed in the third chamber 25180. In some embodiments, a count of the motor(s) may be two, and the two motor(s) may both be disposed in the third chamber 25180, or alternatively, the two motor(s) may be disposed in second chamber 25170 and the third chamber 25180, respectively.

In some embodiments, the noise reduction assembly 25100 may further include one or more shock absorbers (not shown) included in the noise reduction box 25130. The one or more shock absorbers may be disposed at a bottom of the gas pressurization unit 25190, or disposed at two ends of the gas pressurization unit 25190. In some embodiments, the one or more shock absorbers may be configured to absorb shocks or vibrations, which are generated during the running of the gas pressurization unit 25190 or generated by the gas flowing from the two or more chambers.

It should be noted that the descriptions of the noise reduction assembly 25100 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIGS. 26A-26B illustrate an exemplary noise reduction assembly according to some embodiments of the present disclosure. The noise reduction assembly 26200 may be similar to or the same as the noise reduction assembly 25100. FIG. 26A shows a cross-sectional view of a noise reduction box 26205 (from a different angle from FIG. 25B) of the noise reduction assembly 26200. FIG. 26B shows an exemplary flow direction of a gas in the noise reduction box 26205 of the noise reduction assembly 26200. As described above, a noise reduction assembly may include a noise reduction box, one or more separation walls configured to define two or more chambers included in the noise reduction box, and one or more connecting pipes included in the noise reduction box. Specifically, the one or more connecting pipes may be configured to direct the gas to flow between the two or more chambers through the one or more separation walls, so that a noise generated by a flow of the gas may be reduced or eliminated. In some embodiments, the two or more chambers may include three chambers, including the first chamber 26250, the second chamber 26260, and the third chamber 26270 illustrated in FIG. 26A.

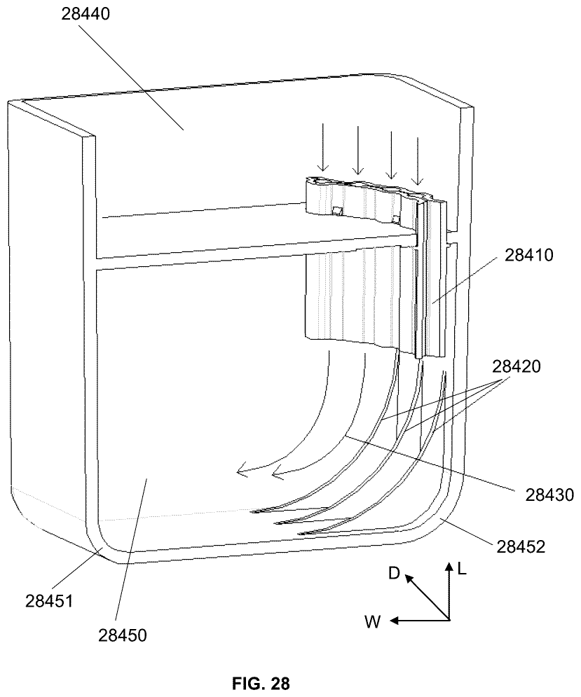

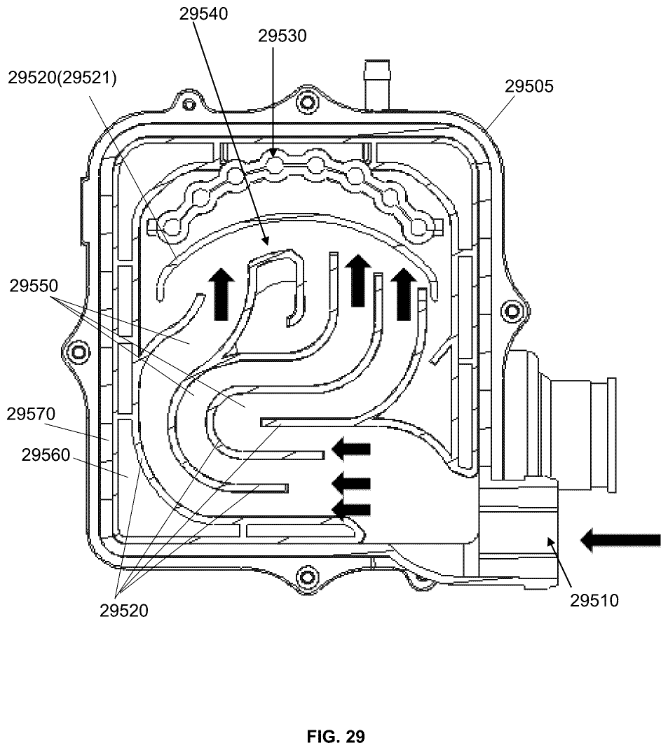

As illustrated in FIGS. 26A and 26B, the gas may enter the first chamber 26250 included in the noise reduction box 26205, and the first chamber 26250 may be configured to accommodate the gas entering the first chamber 26250 via a gas inlet port 26210 of the first chamber 26250. In some embodiments, the first chamber 26250 may include one or more guide ribs (see the guide ribs 28420 in FIG. 28) disposed on an inner wall (e.g., a corner of the inner wall) of the first chamber 26250. The one or more guide ribs may be in a shape of a fusiform slice (or approximate triangle) with a certain thickness, and may be configured to guide the gas to flow. More descriptions of the guide ribs may be found elsewhere in the present disclosure, for example, FIG. 28 and the relevant descriptions, which may not be repeated herein. Further, the first chamber may be divided into or include one or more guide passages defined by one or more guide vanes disposed on an inner wall of the first chamber. In some embodiments, the guide passages may be connected with each other in a fluid communication. Then the gas may be divided into a plurality of portions, and flow in the plurality of guide passages. The noise may thus be blocked or reduced by walls of the plurality of guide passages. More descriptions of structures of the first chamber may be found elsewhere in the present disclosure, for example, FIG. 29 and the relevant descriptions, which may not be repeated herein.

In some embodiments, the one or more connecting pipes may include at least one first pipe 26230 (similar to or the same as the first pipe 25141 in FIG. 25B). The at least one first pipe 26230 may be configured to form a fluid communication between the first chamber 26250 and another chamber of the two or more chambers, to guide the gas to flow from the first chamber 26250 into the another chamber. As illustrated in FIGS. 26A and 26B, most portions of the first pipe 26230 are covered by components (e.g., the gas pressurization unit 26280, the guide passages of the first chamber 26250, or the like) of the noise reduction assembly 26200. In some embodiments, one end of the first pipe 26230 may be disposed in the first chamber 26250, and another end of the first pipe 26230 may be disposed in the another chamber. Thus, the gas may flow from the first chamber to the another chamber via the first pipe 26230.

As illustrated in FIGS. 26A and 26B, the another chamber may include or be the third chamber 26270. Thus, the gas may be divided into a plurality of portions of gas by the guide passages in the first chamber 26250, and flow from the first chamber 26250 to the third chamber 26270 along a direction indicated by two dotted arrows 26201 (see FIG. 26B). The beginning of each of the two dotted arrows 26201 may be in the first chamber 26250, and a direction of each of the two dotted arrows 26201 may point to the third chamber 26270. The at least one first pipe 26230 may be arranged along the direction indicated by the two dotted arrows 26201, thus the at least one first pipe 26230 may be configured to guide the gas to flow from the first chamber 26250 to the third chamber 26270. In some embodiments, the third chamber 26270 may be separated from the first chamber 26250 by the second chamber 26260. Exemplary guide passages may be shown in FIG. 29.

With connection of description of FIGS. 25A and 25B, one or more separation walls may define the two or more chambers, the housing of the noise reduction box 26205 may be divided into two portions, and the gas pressurization unit may be disposed in one or more chambers. In some embodiments, the gas pressurization unit 26280 may be disposed in the third chamber 26270, thus the third chamber 26270 may be defined by a portion of the housing, a shell of the gas pressurization unit 26280, and one of the one or more separation walls. The gas entering into the third chamber 26270 may be capable of cooling the motor of the gas pressurization unit 26280. For example, the gas may flow around the motor of the gas pressurization unit 26280, thus the gas may take away at least a portion of the heat generated during the running of the gas pressurization unit 26280. Thus, the gas pressurization unit 26280 may work under an appropriate temperature. In some embodiments, the appropriate temperature may refer to a temperature that may not cause damage(s) to the gas pressurization unit 26280, and may not be limited in the present disclosure. In some embodiments, the gas may be buffered in the third chamber 26270. That is, the noise generated by the gas flowing may further reduced in the third chamber 26270.

As illustrated in FIGS. 26A and 26B, after the gas flows into the third chamber 26270, the gas may further flow into a second chamber 26260 via at least one another connecting pipe along a direction indicated by the solid arrows 26202 (see FIG. 26B). In some embodiments, the another connecting pipe may be the second pipe 26240. That is, the one or more connecting pipes may further include at least one second pipe 26240, and the at least one second pipe 26240 may be configured to form a fluid communication between the third chamber 26270 and the second chamber 26260, to guide the gas to flow from the third chamber 26270 into the second chamber 26260. The second chamber 26260 may be adjacent to the first chamber 26270, and may separate the first chamber 26250 and the third chamber 26270. In some embodiments, the second chamber 26260 may be a slit disposed between the first chamber 26250 and the third chamber 26270.

After the gas flows into the second chamber 26260, the gas may further flow into the gas pressurization unit 26280 via a gas inlet port (not shown) of the gas pressurization unit 26280. A flow direction of the gas from the second chamber 26260 into the gas pressurization unit 26280 may be indicated by two dotted arrows 26203. The beginning of each of the two dotted arrows 26203 may be in the second chamber 26260, and each of the two dotted arrows 26203 may point to the gas inlet port of the gas pressurization unit 26280. In some embodiments, the gas inlet port of the gas pressurization unit 26280 may open to or in the second chamber 26260, so that the gas entering the second chamber 26260 may be capable of being sucked into the gas pressurization unit 26280 via the gas inlet port of the gas pressurization unit 26280.

As illustrated in FIGS. 26A and 26B, the second chamber 26260 may include a guide structure. The guide structure may be configured to guide the gas to flow into the gas inlet port of the gas pressurization unit 26280. Specifically, the guide structure may include a conical bulge disposed on an inner wall of the second chamber, and may face the gas inlet port of the gas pressurization unit 26280. The more description may be found elsewhere in the present disclosure, for example, FIG. 30 and relevant descriptions, which may not be repeated herein.

A gas outlet port (not shown) of the gas pressurization unit 26280 may be open to an outside of the noise reduction box 26205 to discharge the pressurized gas. As shown in FIG. 26B, the gas may flow into the gas pressurization unit 26280, and be pressurized by the gas pressurization unit 26280. That is, the gas pressurization unit 26280 may drive the gas to flow out of the noise reduction box 26205 along the direction indicated by the hollow arrows 26204 to, for example, the gas mixing assembly 730, or the gas export 204 of the ventilatory treatment apparatus 200.

In some embodiments of the present disclosure, the two or more chambers (e.g., the first chamber 26250, the second chamber 26260, and the third chamber 26270) may be included in the noise reduction box 26205. The noise generated by the gas flowing may be reduced in the two or more chambers with a process of converting sound wave of the noise into heat energy absorbed by the two or more chambers. Further, the gas entering the third chamber 26270 may cool the motor of the gas pressurization unit 26280, thus a service life of the gas pressurization unit 26280 may be lengthened.

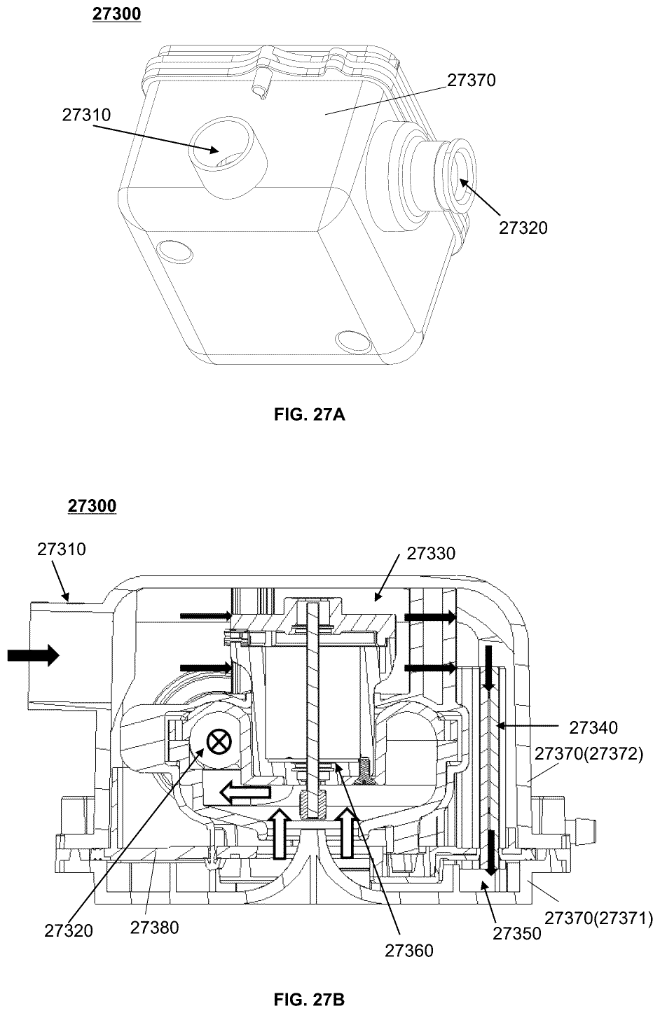

FIGS. 27A-27B illustrate another exemplary noise reduction assembly according to some embodiments of the present disclosure. FIG. 27A shows an overall view of the noise reduction assembly 27300. FIG. 27B shows an exemplary flow direction of the gas in the noise reduction box 27370 of the noise reduction assembly 27300. As described above, the gas entering a first chamber included in a noise reduction box may flow from the first chamber into an another chamber. In some embodiments, the noise reduction box may include three chambers. Thus, the another chamber may be the third chamber, and the gas may flow from the first chamber into the third chamber as illustrated in FIG. 26B.

In some embodiments, the noise reduction box may include only two chambers, including the first chamber and the second chamber. The another chamber may include or be the second chamber. Thus, the gas entering the first chamber may flow from the first chamber into the second chamber. In some embodiments, as a similar way of the noise reduction box 25130 illustrated in FIG. 25A, the housing of the noise reduction box 27370 including two chambers may also be made of plastic, and/or the housing of the noise reduction box 27370 may be divided into two portions, including a lower portion 27371 and an upper portion 27372. The plastic may include a general plastic, an engineering plastic, or a special plastic, which may not be limited in the present disclosure.

As illustrated in FIGS. 27A and 27B, the noise reduction box 27370 may include two chambers, including the first chamber 27330 and the second chamber 27350. The first chamber 27330 and the second chamber 27350 may be separated by a separation wall 27380. the gas may enter the first chamber 27330 via a gas inlet port 27310 of the first chamber 27330 along a direction indicated by the solid arrows. The gas pressurization unit 27360 may be disposed in the first chamber 27330, and a central portion of the gas pressurization unit 27360 may be suspended in the first chamber 27330. Thus, the gas entering into the first chamber 27330 may be capable of cooling a motor of the gas pressurization unit 27360. For example, the gas may flow around the gas pressurization unit 27360, and take away at least a portion of the heat generated during the running of the gas pressurization unit 27360. A gas inlet port (not shown) of the gas pressurization unit 27360 may be open to or in the second chamber 27350, so that the gas entering the second chamber 27350 may be capable of being sucked into the gas pressurization unit 27360 via the gas inlet port of the gas pressurization unit 27360. A gas outlet port 27320 of the gas pressurization unit 27360 may be open to an outside of the noise reduction box 27370 to discharge the pressurized gas. It should be noted that in some embodiments, as shown in FIGS. 25A-27B, the gas pressurization unit may include a shell including a gas inlet port and a gas outlet port. The shell may be wrapped tightly with the gas pressurization unit, the gas inlet port of the shell may be in fluid communication with the gas inlet port of the gas pressurization unit, and the gas outlet port of the shell may be in fluid communication with the gas outlet port of the gas pressurization unit. In the present disclosure, the gas inlet port of the shell may also be referred to as the gas inlet port of the gas pressurization unit, and the gas outlet port of the shell may also be referred to as the gas outlet port of the gas pressurization unit.

In some embodiments, as a similar way of the shell of the gas pressurization unit 25190 illustrated in FIG. 25B, the shell of the gas pressurization unit 27360 may also be made of soft material(s) (e.g., silica gel or soft colloid), so that vibrations generated during the running of the gas pressurization unit 27360 can be absorbed by the soft materials, thereby reducing or eliminating the noise of the gas flowing. Further, the shell of the gas pressurization unit 27360 may form a sealed connection with the separation wall 27380 and/or fix the gas pressurization unit 27360 to a fixed position (e.g., one or more positions on the separation wall 27380, and/or one or more positions on one or more inner walls of the noise reduction box 27370). The fixed position may be determined by a manufacturer of the noise reduction box 27370, which may not be limited in the present disclosure.

At least one connecting pipe 27340 (also referred to as at least one first pipe) may form a fluid communication between the first chamber 27330 and the second chamber 27350, to guide the gas to flow from the first chamber 27330 into the second chamber 27350. After the gas flow through the first chamber 27330, the gas may enter into the second chamber 27350 via the connecting pipe 27340, for example, along an arrangement direction of the connecting pipe 27340 indicated by the solid arrows.

As a similar way of the second chamber 26260 illustrated in FIGS. 26A and 26B, the second chamber 27350 may also include a guide structure configured to guide the gas to flow into the gas inlet port (not shown) of the gas pressurization unit 27360. In some embodiments, the guide structure may include a conical bulge. The conical may be disposed on an inner wall of the second chamber, and may face the gas inlet port of the gas pressurization unit 27360. In some embodiments, the conical bulge may be configured to guide the gas in the second chamber to flow into the gas pressurization unit 27360 via the gas inlet port. The more description of the guide structure may be found elsewhere in the present disclosure, for example, FIG. 30 and relevant descriptions, which may not be repeated herein.

After the gas flows into the second chamber 27350, since the gas pressurization unit 27360 is running, a pressure in the second chamber 27350 may be lower than the standard atmospheric pressure. The gas flowing into the second chamber 27350 may be sucked into the gas pressurization unit 27360 via the gas inlet port of the gas pressurization unit 27360. Because of the action of a pressure difference between the pressure in the second chamber 27350 and the standard atmospheric pressure and running of the gas pressurization unit 27360, the gas sucked into the gas pressurization unit 27360 may be pressurized and discharged outside of the noise reduction box 27370 via the gas outlet port 27320.

In some embodiments of the present disclosure, two chambers (e.g., the first chamber 27330 and the second chamber 27350) may be included in the noise reduction box 27370. Compared with a noise reduction box including three chambers (e.g., the noise reduction box illustrated in FIGS. 25A-25B, or FIGS. 26A-26B), a volume of the noise reduction box may be reduced. The noise generated in the gas inlet port 27310 of the first chamber 27330 may be reduced in the two chambers with a process of converting sound wave of the noise into heat energy absorbed by the two chambers. Further, the gas entering the first chamber 27330 may cool a motor of the gas pressurization unit 27360, thus a service life of the gas pressurization unit 27360 may be lengthened.

It should be noted that the descriptions of the noise reduction box including two chambers are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, an inner diameter of the connecting pipe 27340 may be reduced compared with the one or more connecting pipes illustrated in FIG. 26A. For another example, a count of the connecting pipe 27340 may be increased compared with the first pipe 26230 illustrated in FIG. 26A. Thus, although a count of chambers illustrated in FIGS. 27A and 27B is less than a count of chambers illustrated in FIGS. 26A and 26B, the noise reduction efficiency may not be affected.

FIG. 28 illustrates exemplary guide rib(s) according to some embodiments of the present disclosure.

As described in FIG. 26B or 27B, a gas may flow in two or more chambers in a noise reduction box of a noise reduction assembly. In some embodiments, at least one chamber of the two or more chambers may include or be equipped with one or more guide ribs. The guide rib(s) may be configured to guide the gas to flow. In some embodiments, an inner wall of at least one corner of the at least one chamber may be arc-shaped to guide the gas to flow.

An exemplary corner of a chamber is shown in FIG. 28. As illustrated in FIG. 28, the gas 28430 may be guided from one chamber 28440 to another chamber 28450 via a connecting pipe 28410 (e.g., from the first chamber 25160 to the third chamber 25180 via the first pipe 25141, from the third chamber 25180 to the second chamber 25170 via the second pipe 25142, from the first chamber 27330 to the second chamber 27350 via the connecting pipe 27340, or the like). An inner wall of a corner (e.g., the corner 28451, the corner 28452, etc.) of the chamber 28450 may be arc-shaped, thus the gas 28430 entering the chamber 28450 may be guided to flow through the chamber 28450 along the arc-shaped corner. In some embodiments, one or more guide ribs 28420 may be disposed on the inner wall of the chamber 28450. When the gas 28430 flows out from outlet port(s) of the connecting pipe 28410 and into the chamber 28450, the guide ribs 28420 may assist to guide the gas to flow, thus the gas 28430 may flow more smoothly in the chamber 28450, and/or the gas resistance of the gas flow may be reduced. In some embodiments, a count of the guide ribs 28420 in at least one chamber may be in a range of 1-6 (e.g., 2-6). In some embodiments, the one or more guide ribs may be disposed in any position in the at least one chamber except those positions where the gas may be blocked when the gas is flowing. In some embodiments, the chamber 28410 may be equipped with or include one or more guide ribs (not shown).

In some embodiments, the guide rib(s) 28420 may be configured as a slice structure with a certain size. In some embodiments, sizes of the guide ribs 28420 may be the same. Alternatively, sizes of the guide ribs 28420 may be different. For example, width(s), and/or length(s) of the guide ribs 28420 disposed in edge positions may be greater than that of the guide ribs 28420 disposed in a medium position. Thus, the gas may be gathered in the medium position, and turbulences generated in the gas 28430 flowing in the chamber 28450 may be reduced, and the noised generated by the turbulences may be reduced. The width of a guide rib 28420 may refer to the length of the guide rib 28420 in the W direction (e.g., parallel to the bottom of the chamber 28450), the length of the guide rib 28420 may refer to a first length of the guide rib 28420 in the L direction (e.g., vertical to the bottom of the chamber 28450) or a second length of the guide rib 28420 in the D direction (e.g., the diagonal direction (parallel to or in a plane of the W direction and the L direction) of the chamber 28450).

It should be noted that the descriptions of the guide ribs are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 29 illustrates exemplary guide passages in a chamber of a noise reduction box according to some embodiments of the present disclosure. In some embodiments, at least one chamber of two or more chambers included in the noise reduction box may include one or more guide ribs. The guide rib may be configured to guide the gas to flow. An inner wall of at least one corner of the at least one chamber may be arc-shaped to guide the gas to flow. In some embodiments, the at least one chamber of the two or more chambers may further include one or more guide passages. The one or more guide passages may be configured to guide the gas to flow in the at least one chamber.

As illustrated in FIG. 29, a first chamber 29505 of a noise reduction box may include one or more guide ribs (not shown). The first chamber 29505 may be the same as or similar to the first chamber 25160, the first chamber 26250, or the first chamber 27330. The guide ribs may guide the gas entering the first chamber 29505 to flow into a connecting pipe 29530 (also referred to as a first pipe). In some embodiments, the one or more guide ribs may be arranged on one or more corners of the first chamber 29505 or another chamber of the two or more chambers included in the noise reduction box. More description of the one or more guide ribs may be found elsewhere in the present disclosure, for example, FIG. 28 and relevant descriptions, which may not be repeated herein. An inner wall of at least one corner of the first chamber 29505 may be arc-shaped, thus the gas may be guided to flow smoothly in the first chamber 29505. In some embodiments, a curvature of the arc-shaped inner wall of the at least one corner of the first chamber 29505 may be determined based on actual requirements of the noise reduction or experience, which may not be limited in the present disclosure.

The first chamber 29505 may further include one or more guide passages 29550 configured to guide the gas to flow in the first chamber 29505. In some embodiments, the one or more guide passages 29550 may be defined by one or more guide vanes 29520 disposed on one or more inner walls of the first chamber 29505 (e.g., a separation wall between the first chamber 29505 and a second chamber). In some embodiments, a gas inlet port 29510 of the first chamber 29505 and the connecting pipe 29530 may be arranged in different sides in the first chamber, and noise generated in the gas inlet port 29510 of the first chamber 29505 may be reduced with the equipment of the guide passage(s), thus the noise may be absorbed by the inner wall of the first chamber 29505 and/or the one or more guide vanes 29520. In some embodiments, the guide passage(s) 29550 may be set as long as possible to reduce the noise as much as possible. Thus, the guide passage(s) 29550 may be curved (e.g., winding). In some embodiments, a curvature of each of the guide passage(s) 29550 of the first chamber 29505 may be determined based on actual requirements of the noise reduction or experience. For example, the greater the curvature of a guide passage is, the more the noise may be absorbed or reduced at a turn of the guide passage. However, the greater the curvature of a guide passage is, the greater the resistance for the gas flowing in the guide passage may be. Thus, the curvature of each of the guide passage(s) of the first chamber 29505 may be determined based on actual requirements of the noise reduction (e.g., a size of the noise reduction box, or the like), which may not be limited in the present disclosure.

As illustrated in FIG. 29, a gas may enter the first chamber 29505 via a gas inlet port 29510 of the first chamber 29505. Then, the gas may be divided into a plurality of portions (also referred to a plurality of gases) flowing into the one or more guide passages 29550 in the first chamber 29505. In some embodiments, the one or more guide vanes 29520 may be positioned to make the gases flowing into the one or more guide passages 29550 have a substantially uniform or identical speed. In some embodiments, the guide passage(s) 29550 may have a same width (i.e., the guide passage(s) 29550 may be arranged in the first chamber 29505 evenly), so that the gases flowing into the guide passage(s) 29550 have a substantially uniform or identical speed.

In some embodiments, a count of the guide passages 29550 may be determined based on the actual requirements of the noise reduction or experience. As described above, the noise may be absorbed by the one or more guide vanes 29520, which may define the one or more guide passages. In some embodiments, although the guide passages 29550 may have a same width, a width from an inner side wall 29570 of the first chamber 29505 to an outermost guide vane of the guide passage(s) 29550 may be greater or less than the width of the guide passage(s) 29550. If gas flows into the space (e.g., the space 29560) between the inner side wall 29570 of the first chamber 29505 and the outermost guide vane, a relatively great noise may be generated, because the speed of the gas flowing therebetween may be different from the speed of the gas flowing in the guide passage(s) 29550 (since the width from the inner side wall 29570 of the first chamber 29505 to the outermost guide vane is different from the width of the guide passage(s) 29550). The greater the difference of those speeds is, the more the noise may be in the first chamber 29505. In some embodiments, the space between the inner side wall 29570 of the first chamber 29505 and the outermost guide vane may be sealed to prevent gas from entering. In some embodiments, a plurality of guide passages 29550 may be arranged in the first chamber 29505 to decrease the difference between the widths, and accordingly, a difference between the speeds of gases may be decreased. That is, the more guide passages are, the more noise may be reduced. However, the count of the passage may be limited with a size of the noise reduction box and/or a forging process of the noise reduction box. As shown in FIG. 29, an exemplary count of the guide passage may be three.

In some embodiments, the first chamber 29505 may include one or more corners 29540 defined by the one or more guide vanes 29520 disposed on one or more inner walls of the first chamber 29505. The one or more corners 29540 may be configured to prevent the noise from spreading out of the noise reduction assembly. For example, the noise may need to be reflected with a lot of times to spread out of the corner(s) 29540, and an energy of the noise may be absorbed by the guide vane(s) 29520. The gases flowing in the guide passage(s) may not be prevented by the corner(s) 29540 since the gases may skip over the corner(s) 29540. However, in some embodiments, the corner(s) 29540 may affect speeds of the gases, thus the corner(s) 29540 may not be arranged or disposed on an end of the guide passage(s) 29550. In some embodiments, the corner(s) 29540 may be arranged or disposed outside the turn(s) of the guide passage(s). In some embodiments, the corner(s) 29540 may be arranged or disposed outside a guide passage closest to the inner side wall 29570 of the first chamber 29505. In some embodiments, a count of the corner(s) 29540 may be determined based on actual requirements of the noise reduction or experience. For example, a count of the corner(s) 29540 may be determined based on a size of the noise reduction box. As another example, there may be no corner 29540 disposed in the first chamber 29505.

As illustrated in FIG. 29, after a plurality of gases flowing through the guide passage(s) 29550, the gas(es) may further skip over a guide vane 29521, and then flow into the connecting pipe 29530 to be guided into another chamber of the noise reduction box. The guide vane 29520 may be configured to guide the gas(es) to flow into the connecting pipe 29530. In some embodiments, a cross section of the guide vane 29521 may have an inclined plane. For example, a first edge (close to the guide passage(s) 29550) of the guide vane 29521 may be relatively lower than a second edge (close to the connecting pipe 29530) of the guide vane 29521, so that the inclined plane may facilitate the gas(es) to flow into the connecting pipe 29530.

In some embodiments of the present disclosure, the guide passage(s) 29550 may be winding, and may be disposed in the first chamber 29505 evenly. There may be no bulge or concavity on the one or more guide vanes 29520 that define the guide passage(s) 29550, thus the gas may be divided into a plurality of gases with a substantially uniform speed, and may flow in the one or more guide passages smoothly. Thus, a turbulence may not be generated because of the gases with the substantially uniform speed, and noise generated by the turbulence may be reduced. In addition, the corner(s) 29540 defined by the guide vane(s) 29520 may prevent the noise from spreading out of the noise reduction assembly. Thus, noise generated in the gas inlet port of the first chamber or generated in gas flowing may be reflected a lot of times by inner wall(s) of the corner(s) 29540, energy of the noise may thus be consumed, and the noise may be reduced.

In some embodiments, the guide vane(s) 29520 may be made of a material that is the same as that of the noise reduction box. In some embodiments, the guide vane(s) 29520 and one or more inner walls of the noise reduction box may be configured as an integral piece.

It should be noted that the descriptions of the structures of the first chamber are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 30 is an exploded view of an exemplary noise reduction assembly according to some embodiments of the present disclosure. The noise reduction assembly 30600 may be the same as or similar to the noise reduction assembly 25100 and/or the noise reduction assembly 26200.

As illustrated in FIG. 30, the noise reduction box 30610 of the noise reduction assembly 30600 may include two portions (e.g., a lower portion 30611 and an upper portion 30612). When the upper portion 30612 is buckled on the lower portion 30610, a shell of the noise reduction box 30610 may be formed. The shell of the noise reduction box 30610 may accommodate two separation walls 30620 and 30640, a connecting pipe 30630 (also referred to as a first pipe), a connecting pipe 30650 (also referred to as a second pipe), and a gas pressurization unit 30670. In some embodiments, the components (e.g., the two separation walls 30620 and 30640, the connecting pipe 30630, the connecting pipe 30650, the gas pressurization unit 30670, or the like) accommodated in the noise reduction box 30610 may be detachable from each other.

The lower portion 30611 may define a first chamber with the separation wall 620. The upper portion 30612 may define a third chamber with the separation wall 30640. Thus, the gas pressurization unit 30670 may be disposed in the third chamber, and a central portion of the gas pressurization unit 30670 may be suspended in the third chamber. In order to fix the gas pressurization unit 30670 in the third chamber, one or more grooves 30641 and/or one or more limiting holes 30642 may be disposed on the separation wall 30640. The groove(s) 30641 (and/or the limiting hole(s) 30642) may be configured to fix or limit the stand bars 30671 (and/or the limiting post(s) 30672) of the gas pressurization unit 30670 (or the shell of the gas pressurization unit 30670). In some embodiments, a count of the groove(s) 30641 (or limiting hole(s) 30642) may correspond to a count of the stand bars 30671 (or limiting post(s) 30672) of the gas pressurization unit 30670. For example, the gas pressurization unit 30670 may include three stand bars 30671 (or limiting post(s) 30672), and three grooves 30641 (or limiting hole(s) 30642) corresponding to the three stand bars (or limiting post(s)) may be disposed on the separation wall 30640 to buckle the three stand bars 30671 (or limiting post(s) 30672). A gas inlet port of the gas pressurization unit 30670 may be open to or in the second chamber, so that the gas entering the second chamber may be capable of being sucked into the gas pressurization unit 30670 via the gas inlet port of the gas pressurization unit 30670.

The second chamber may be a slit defined by the two separation walls 30620 and 30640. Thus, the second chamber may separate the first chamber and the third chamber. The second chamber may include a guide structure configured to guide the gas to flow into the gas inlet port of the gas pressurization unit 30670. As illustrated in FIG. 30, the guide structure may include a conical bulge 30621 disposed on an inner wall of the second chamber (or disposed on the separation wall 30620). The conical bulge 30621 may face the gas inlet port of the gas pressurization unit 30670. That is, a pointed end of the conical bulge 30621 may face the gas inlet port of the gas pressurization unit 30670. Since the conical bulge 30621 may have a shape of a cone, the gas may be guided to flow into the gas pressurization unit 30670 via the conical bulge 30621 smoothly, and no turbulence may be generated in the gas inlet port of the gas pressurization unit 30670, thus the noise generated in the gas inlet port of the gas pressurization unit 30670 may be reduced. In some embodiments, a certain distance between the pointed end of the conical bulge 30621 and the gas inlet port of the gas pressurization unit 30670 may be provided. For example, the distance may be 2 mm. In some embodiments, the distance may be determined based on actual requirements of the noise reduction or experience, which may not be limited in the present disclosure. In some embodiments, in order to ensure that the gas in the second chamber be sucked into the gas pressurization unit 30670 smoothly, a diameter of a conical bottom of the conical bulge 30621 may be the same as or greater than a diameter of a fan (or motor) in the gas pressurization unit 30670. In some embodiments, a projection of the conical bottom of the conical bulge 30621 and a projection of the gas inlet port of the gas pressurization unit 30670 may be overlapped and may form a shape of a ring. The projection may refer to the projection onto the separation wall 30620 or a plane parallel to the separation wall 30620. In order to ensure that the gas in the second chamber be sucked into the gas pressurization unit 30670 smoothly, a width of the ring may be greater than 6 mm. For example, a diameter of the fan (or motor) in the gas pressurization unit 30670 may be more than 6 mm greater than a diameter of the gas inlet port of the gas pressurization unit 30670. In some embodiments, an area of the ring may be greater than an area of the projection of the gas inlet port of the gas pressurization unit 30670.

The first pipe 30630 may pass through the two separation walls 30620 and 30640, thus the first chamber may form a fluid communication with the third chamber, to guide the gas to flow from the first chamber into the third chamber. In order to allow the first pipe 30630 to pass through, through holes may be provided on the two separation walls 30620 and 30640, respectively. The second pipe 30650 may be disposed between the third chamber and the second chamber, and pass through the separation wall 30640, thereby forming a fluid communication between the third chamber and the second chamber, to guide the gas to flow from the third chamber into the second chamber. In some embodiments, the noise reduction assembly 30600 may further include one or more position-limit mechanisms. The position-limit mechanism(s) may be configured to assist to mount the connecting pipe(s) onto the separation wall(s). For example, a first position-limit mechanism may assist to mount the first pipe 30630 onto the separation wall 30620 and/or the separation wall 30640. As another example, a second position-limit mechanism may assist to mount the second pipe 30650 onto the separation wall 30640. A way of mounting the second pipe 30650 may be similar to the same as the way of mounting the first pipe 30630. More descriptions of mounting the connecting pipes (e.g., the first pipe 30630 or the second pipe 30650) and the position-limit mechanism may be found elsewhere in the present disclosure, for example, FIG. 32A and relevant descriptions, which may not be repeated herein.

As illustrated in FIG. 30, the noise reduction assembly 30600 may be configured without a sound absorbing cotton. If a sound absorbing cotton is used in a noise reduction assembly, and the sound absorbing cotton is used for a long time, or is sterilized by ozone, particles may be produced from the sound absorbing cotton, and the particles may be spread into the gas. Since no sound absorbing cotton is provided in the noise reduction assembly 30600, particles in a pressurized gas supplied by the gas pressurization unit 30670 may be reduced, thus the quality of the pressurized gas may be improved.

It should be noted that the descriptions of the structures of the noise reduction box are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIGS. 31A-31B illustrate an exemplary connecting pipe according to some embodiments of the present disclosure. FIG. 31A shows an overview of the connecting pipe 31710. FIG. 31B shows a cross-sectional view of the connecting pipe 31710. In a noise reduction box of a noise reduction assembly, at least one connecting pipe may be disposed between two or more adjacent chambers, and may be mounted on a separation wall between the two or more chambers through a position-limit mechanism. Thus, a chamber in the noise reduction box may be in a fluid communication with another chamber via the connecting pipe.

As illustrated in FIG. 31A, a through hole 31730 may be provided on a separation wall 31720, thus the connecting pipe 31710 may be mounted on the separation wall 31720 via the through hole 31730. The connecting pipe 31710 may include one or more sub-pipes 31715. In some embodiments, each sub-pipe of the one or more sub-pipes 31715 may be connected with at least one another sub-pipe of the one or more sub-pipes 31715 along a certain direction (e.g., a radial direction of the each sub-pipe). In some embodiments, a count of the one or more sub-pipes 31715 may be 8. In some embodiments, the count of the one or more sub-pipes 31715 may be determined based on actual requirements of the noise reduction or experience, which may not be limited in the present disclosure. A shape of the through hole 31730 may match an outline of the connecting pipe 31710. In some embodiments, the connecting pipe 31710 may be mounted on the separation wall 31720 between two chambers through (or with an assist of) a position-limit mechanism. In some embodiments, the shape of the through hole 31730 may further need to match the position-limit mechanism. More descriptions of the position-limit mechanism may be found elsewhere in the present disclosure, for example, FIG. 32A and the relevant descriptions, which may not be repeated herein.

After the connecting pipe 31710 is mounted on the separation wall 31720 between two chambers, a gas in a chamber may flow into another chamber via the connecting pipe 31710. Specifically, the connecting pipe 31710 (or the sub-pipe(s) 31715) may have a hollow structure, and the gas may pass through the connecting pipe 31710 via the hollow portion 31750 in the connecting pipe 31710 illustrated in FIG. 31B. As described above, the connecting pipe 31710 may be formed by one or more sub-pipes 31715 connected with each other. In some embodiments, the one or more sub-pipes 31715 may have a circular cross section. At least one sub-pipe of the one or more sub-pipes 31715 may be in fluid communication with another sub-pipe of the one or more sub-pipes 31715 via a channel along a certain direction (e.g., a radial direction of the at least one sub-pipe 31715). That is, a hollow portion 31750 of the at least one sub-pipe of the one or more sub-pipes 31715 may be connected with a hollow portion 31750 of another sub-pipe of the one or more sub-pipes 31715 via a hollow portion 31740 of the channel as illustrated in FIG. 31B. A width of the channel may affect the effect of noise reduction and a resistance for gas flowing. In some embodiments, the width of the channel may be smaller than a maximum width (e.g., a diameter) of the at least one sub-pipe.

A diameter of at least one sub-pipe may also affect the effect of noise reduction and a resistance for gas flowing. The greater the diameter of a sub-pipe is, the less the resistance for gas flowing may be, but the worse the effect of the noise reduction may be. In some embodiment, a diameter of a sub-pipe may not be greater than 6 mm. For example, the diameter of at least one sub-pipe may be 5.5 mm. In some embodiments, the one or more sub-pipes 31715 may have substantially uniform (or identical) diameters. For example, diameters of the one or more sub-pipes 31715 may be the same. In some embodiments, the one or more sub-pipes 31715 may have un-uniform (or unidentical) diameters. For example, the diameters of all of the one or more sub-pipes 31715 may be different. For another example, diameters of a portion of the one or more sub-pipes 31715 may be the same, but diameters of another portion of the one or more sub-pipes 31715 may be different from one another.

In some embodiments of the present disclosure, at least one sub-pipe of the one or more sub-pipes 31715 may be in fluid communication with another sub-pipe of the one or more sub-pipes 31715 via a channel. In some embodiments, the hollow portion of the connecting pipe (i.e., the hollow portion 31750 of the sub-pipe(s) 31715, and the hollow portion 31740 of the channel) may be regarded as a whole. Thus, a resistance for the gas flowing may be reduced, and when the gas passes through the connecting pipe 31710, speeds of different portions of the gas in different portions (e.g., different sub-pipe(s) 31715) of the connecting pipe 31710 may be substantially uniform. Thus, the gas may flow from a chamber into another chamber stably, and the uniform speed may cause less noise when the gas flowing in the connecting pipe 31710.

FIGS. 32A-32C illustrate an exemplary connecting pipe and an exemplary position-limit mechanism according to some embodiments of the present disclosure. FIG. 32A shows an overall view of the connecting pipe 32810 and the position-limit mechanism 32820. FIG. 32B shows exemplary pieces of the connecting pipe 32810. FIG. 32C shows a cross-sectional view of the connecting pipe 32810. As described above, at least one connecting pipe may be disposed between two or more adjacent chambers of the two or more chambers, and may be mounted on a separation wall between the two or more adjacent chambers through a position-limit mechanism.

As illustrated in FIG. 32A, the position-limit mechanism 32820 may include a flange 32821. The flange 32821 may be configured to assist the connecting pipe 32810 to match an inner wall of at least one chamber or a separation wall between two chambers. For example, the flange 32821 may provide a hole matching an outline of the connecting pipe 32810, and the flange 32821 may be fixed on the separation wall between two chambers, thus the flange 32821 may limit a position of the connecting pipe 32810 on the separation wall. In some embodiments, the shape of a through hole (e.g., the through hole 31730 illustrated in FIG. 7) provided on the separation wall may match the outline of the connecting pipe 32810, thus the connecting pipe 32810 may be fixed on the separation wall tightly. Then, the flange 32821 may further limit a position of the connecting pipe 32810, thus assisting the connecting pipe 32810 fixed on the separation wall more tightly. In some embodiments, there may be no space between the connecting pipe 32810 and the separation wall (or an inner wall of a chamber), thus the gas may not flow from a chamber to another chamber via the through hole used for fixing the connecting pipe 32810.

The position-limit mechanism 32820 may further include one or more snaps 32822 disposed on the connecting pipe 32810. The one or more snaps 32822 may be configured to fix the connecting pipe 32810 on the inner wall of at least one chamber or the separation wall between two chambers. In some embodiments, the flange 32821 and the snap(s) 32822 may be separated by a certain distance, so that (when the connecting pipe 32810 is mounted on the separation wall between two chambers) the flange 32821 may be positioned on one side of the separation wall to limit a position of the connecting pipe 32810, and the one or more snaps 32822 may be positioned on another side of the separation wall to buckle the connecting pipe 32810 on the separation wall. That is, the position-limit mechanism 32820 may include separate structures, and the one or more snaps 32822 may be separated from the flange 32821 by the separation wall. In some embodiments, the separation distance between the flange 32821 and the snap(s) 32822 may be substantially equal to the thickness of the separation wall.

In some embodiments, the at least one connecting pipe (e.g., the connecting pipe 32810) may be configured as an integral piece. In some embodiment, the at least one connecting pipe (e.g., the connecting pipe 32810) may also include separate structures. As illustrated in FIG. 32B, the connecting pipe 32810 may be assembled using two or more pieces. The two or more pieces may form the connecting pipe 32810 by a way of welding or buckling with snaps.

In some embodiments, as illustrated in FIGS. 32B-32C, the connecting pipe 32810 may include one or more strut members 32840 disposed in one or more channels 32860 (e.g., the channel illustrated in FIG. 31B). The strut member 32840 may be configured to prevent the channel from deformation. As described above, the connecting pipe 32810 may be fixed on the separation wall (e.g., a separation wall 32850 illustrated in FIG. 32C) tightly with the flange 32821. Thus, an outer surface of the connecting pipe 32810 may not be deformed because of a limitation from the flange 32821 and/or the separation wall 32850. Meanwhile, an inner surface (e.g., an inner surface of the channel(s) 32860) may not be deformed because of the support from the strut member(s) 32840. In some embodiments, each channel of the channel(s) 32860 may be equipped with a strut member 32840. In some embodiments, one or more of the channel(s) 32860 may be equipped with no strut member.

In some embodiments, at least one end of at least one connecting pipe (e.g., the connecting pipe 32810 illustrated in FIG. 32A or 32C) may be beveled, thus a range of a wavelength of noise that may be reduced in the connecting pipe may be widened. The effect of noise reduction of a connecting pipe with a beveled end may be better than the effect of noise reduction of a connecting pipe with non-beveled end. In some embodiments, the at least one end of the at least one connecting pipe may be beveled to facilitate the gas to flow. For example, with connection of description of FIG. 26A or 26B, a gas pressurization unit may be disposed in a chamber of a noise reduction box. A gas entering into the chamber may be capable of cooling a motor of the gas pressurization unit. A beveled end of the connecting pipe may guide the gas flowing into the gas pressurization unit more smoothly. In some embodiment, the beveled end may face the gas pressurization unit. That is, the length of a side (or a piece) of the connecting pipe near the gas pressurization unit may be shorter than the length of another side (or another piece) of the connecting pipe away from the gas pressurization unit. As another example, with connection of FIG. 29, a beveled end of the connecting pipe 29530 may face the guide passage(s) 29550 (e.g., the length of a side (or a piece) of the connecting pipe 29530 near the guide passage(s) 29550 may be shorter than the length of another side (or another piece) of the connecting pipe 29530 away from the guide passage(s) 29550).

The energy of noise generated in a process of gas flowing may be consumed in the connecting pipe 32810. That is, the longer the connecting pipe is, the better the effect of noise reduction may be. In some embodiments, the length of at least one pipe of one or more connecting pipes in a noise reduction box may be greater than 25 mm. An actual length of a connecting pipe may be determined based a size of the noise reduction box and actual requirements of the noise reduction, which may not be repeated herein.

It should be noted that the descriptions of the structures of the connecting pipe are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

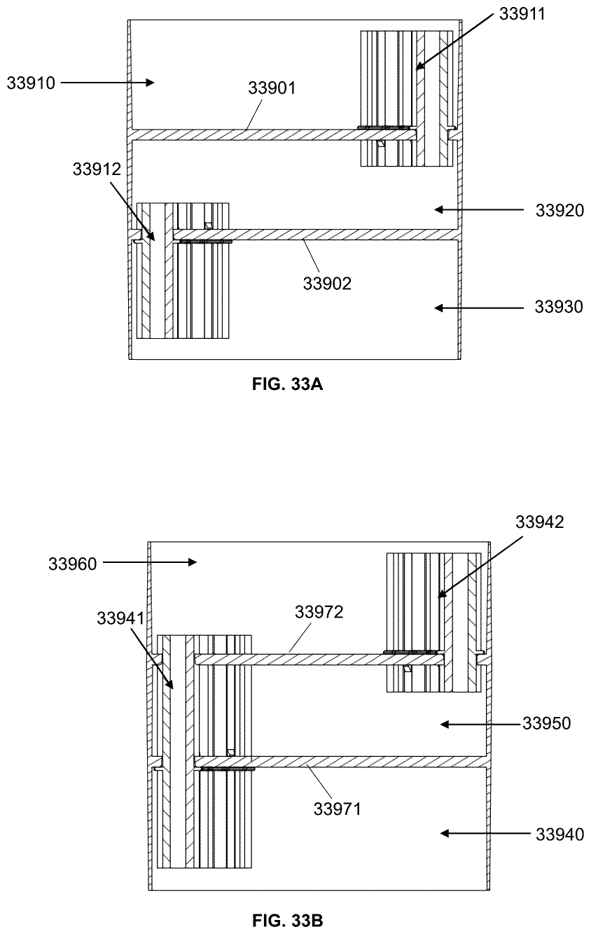

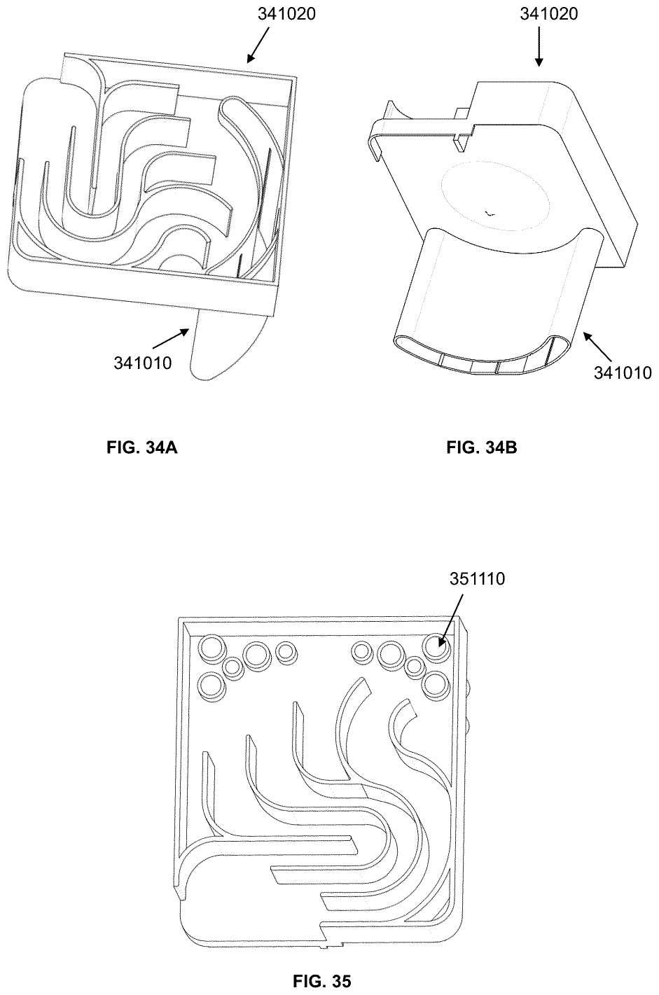

FIGS. 33A-33B illustrate exemplary distribution of two or more chambers and one or more connecting pipes in a noise reduction box according to some embodiments of the present disclosure. As described above, a noise reduction assembly may include a noise reduction box, one or more separation walls, and one or more connecting pipes. The noise reduction box may include two or more chambers. For example, the noise reduction box may include two chambers as illustrated in FIGS. 27A-27B. As another example, the noise reduction box may include three chambers as illustrated in FIGS. 25B-26B.

In some embodiments, two adjacent chambers of the two or more chambers in the noise reduction box may be connected with a separation wall, and the two adjacent chambers may be in fluid communication via a connecting pipe that passes through the separation wall. As illustrated in FIG. 33A, a first chamber 33910 may be separated from a second chamber 33920 by a separation wall 33901. A connecting pipe 33911 may pass through the separation wall 33901, and the first chamber 33910 and the second chamber 33920 may be in fluid communication through the connecting pipe 33911. A gas may flow from the first chamber 33910 into the second chamber 33920 via the connecting pipe 33911. As a similar way, the second chamber 33920 may be separated from the third chamber 33930 by a separation wall 33902, but the second chamber 33920 may be in fluid communication with the third chamber 33930 through the connecting pipe 33912. Therefore, the first chamber 33910 may be in fluid communication with the third chamber 33930 through the connecting pipes 33911 and 33912. However, the first chamber 33910 may not be in fluid communication with the third chamber directly.

In some embodiments, two adjacent chambers of the two or more chambers in the noise reduction box may not be in fluid communication. As illustrated in FIG. 33B, a first chamber 33940 may be separated from a second chamber 33950 by a separation wall 33971, the second chamber 33950 may be separated from a third chamber 33960 by a separation wall 33972, and the third chamber 33960 may be separated from the first chamber 33940 by the second chamber 33950. A gas in the first chamber 33940 may not flow into the second chamber 33950 directly. In contrast, the gas in the first chamber 33940 may flow into the third chamber 33960 via a connecting pipe 33941. Then, the gas entering the third chamber 33960 may flow into the second chamber 33950 via another connecting pipe 33942.

In some embodiments, the two or more chambers may not be disposed in the noise reduction box with a constant order. For example, an adjacent chamber of the first chamber may be the second chamber. As another example, an adjacent chamber of the first chamber may be the third chamber.

It should be noted that the descriptions of the distribution of one more connecting pipes in the two or more chambers are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIGS. 34A-34B illustrate another exemplary connecting pipe viewing from two different sides of a separation wall according to some embodiments of the present disclosure. As described above, a connecting pipe may be configured to direct a gas to flow between two or more chambers through one or more separation walls.

As illustrated in FIGS. 34A-34B, a connecting pipe 341010 may pass through a separation wall 341020. One side of the separation wall 341020 may be or form an inner wall of a chamber, and another side of the separation wall 341020 may be or form an inner wall of another chamber. Thus, a gas flowing into the chamber may be directed to flow from the chamber to the another chamber though the connecting pipe 341010.

In some embodiments, one or more guide passages defined by one or more guide vanes may be disposed on the one side of the separation wall 341020 (i.e., the inner wall of the chamber) to guide the gas to flow in the chamber. After flowing through the chamber, the gas may be guided to flow into the another chamber. In some embodiments, a height of a portion of the connecting pipe 341010 in the chamber may be less than or equal to a height of a guide vane, thus the gas may flow from the one or more guiding passages into the connecting pipe 341010 smoothly. In some embodiments, the height of a portion of the connecting pipe 341010 in the chamber may be in an range of 5 mm-15 mm. In some embodiments, the height of a portion of the connecting pipe 341010 in the chamber may be determined based on principles of hydrodynamics, which may not be limited in the present disclosure.

In some embodiments, a section view of the connecting pipe 341010 may be a hollow strip (e.g., a curved hollow strip). That is, the connecting pipe 341010 may be designated as a whole pipe with a smooth inner wall (i.e., there may be no bulge provided on the inner wall of the connecting pipe 341010). In some embodiments, the connecting pipe 341010 may be formed as a whole by forging or injection molding. In some embodiments, the connecting pipe 341010 and the separation wall 341020 may be configured as an integral piece. In some embodiments, the connecting pipe 341010 may be assembled using two or more smooth curve pieces.

It should be noted that the descriptions of structures of the connecting pipe 341010 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 35 illustrates another exemplary connecting pipe according to some embodiments of the present disclosure.

As illustrated in FIG. 35, a plurality of connecting pipes 351110 may be disposed on a separation wall. In some embodiments, the separation wall may be configured to form a portion of two chambers separated by the separation wall, the plurality of connecting pipes 351110 may pass through the separation wall to guide a gas to flow from a chamber into another chamber of the two chambers.

In some embodiments, each of the plurality of connecting pipes 351110 may be independent from others. That is, the plurality of connecting pipes 351110 may be disposed on the separation wall dispersedly. In some embodiments, the plurality of connecting pipes 351110 may be disposed on the same side of the separation wall. In some embodiments, the plurality of connecting pipes 351110 may be disposed on different sides of the separation wall.

In some embodiments, the section view of each of the plurality of connecting pipes 351110 may be a circle or an approximate circle. In some embodiments, inner diameters of the plurality of connecting pipes 351110 may be the same. In some embodiments, inner diameters of the plurality of connecting pipes may be different. In some embodiments, the inner diameters of the plurality of connecting pipes may be determined based on actual requirements of gas flowing or principles of the hydrodynamics, which may not be limited in the present disclosure.

It should be noted that the descriptions of structures of the connecting pipe 351110 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

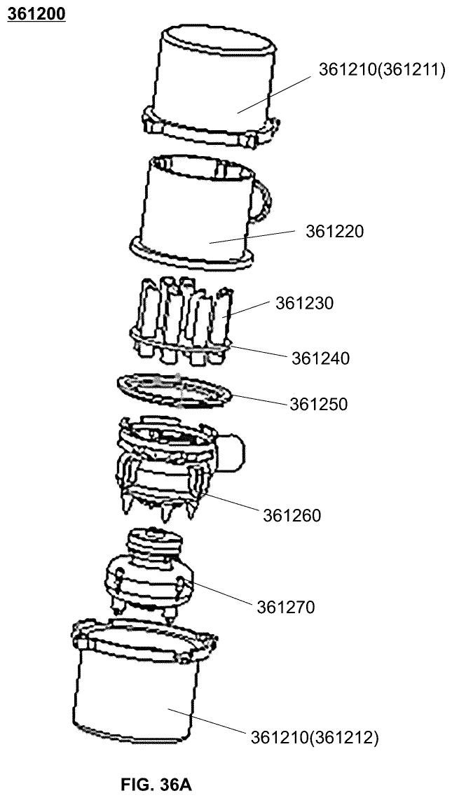

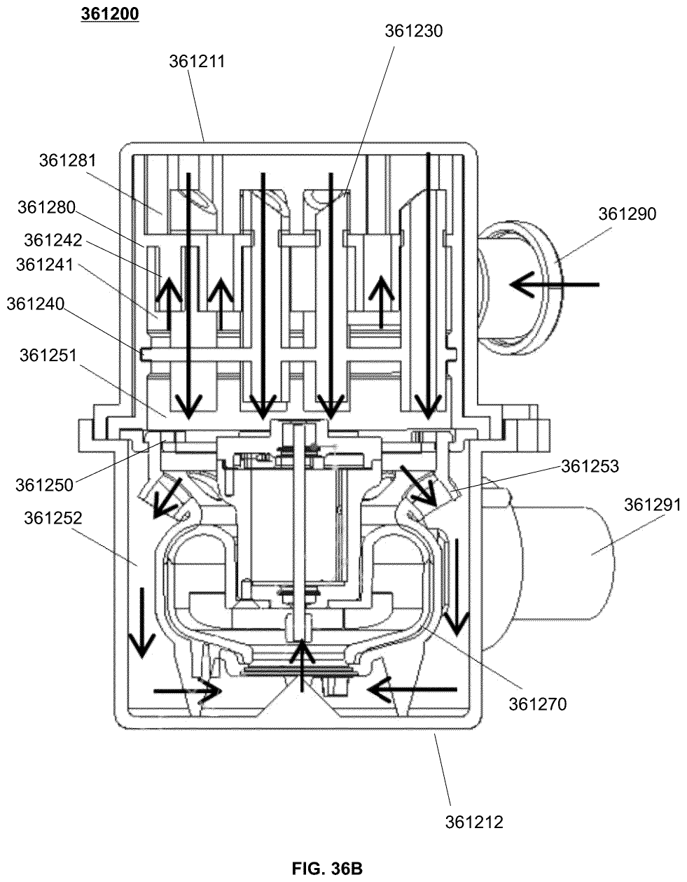

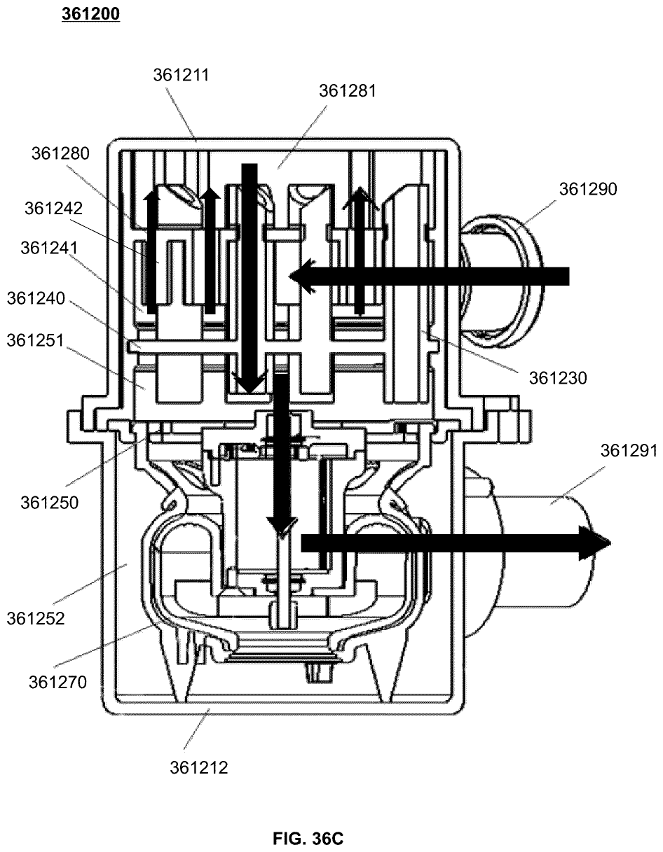

FIGS. 36A-36B illustrate another exemplary noise reduction assembly according to some embodiments of the present disclosure. FIG. 36A shows an exploded view of the noise reduction assembly 361200. FIG. 36B shows an exemplary flow direction of a gas in a noise reduction box of the noise reduction assembly 361200. FIG. 36C shows another exemplary flow direction of a gas in a noise reduction box of the noise reduction assembly 361200.

As illustrated in FIG. 36A, the noise reduction box 361210 of the noise reduction assembly 361200 may include two portions (e.g., an upper portion 361211 and a lower portion 361212). When the upper portion 361211 is buckled on the lower portion 361212, a shell of the noise reduction box 361210 may be formed. In some embodiments, a connecting pipe (also referred to as a first pipe shown in FIGS. 36B and 36C) and a separation wall (also referred to as a first separation wall shown in FIGS. 36B and 36C) are disposed in the upper portion 361211 of the noise reduction box 361210. In some embodiments, the shape of a shell of the noise reduction box 361210 may be a cylinder. The shell of the noise reduction box 361210 may accommodate a support structure 361220, a connecting pipe 361230 (also referred to as a second pipe), two separation walls 361240 and 361250 (also referred to as a second separation wall and a third separation wall, respectively), a shell 361260 of a gas pressurization unit 361270, and a gas pressurization unit 361270. In some embodiments, a portion of the components (e.g., the support structure 361220, the connecting pipe 361230, two separation walls 361240 and 361250) accommodated in the noise reduction box 361210 may be detachable from each other.

In some embodiments, the connecting pipe 361230 may include a plurality of connecting pipes dispersed from each other, and the plurality of connecting pipes may be fixed on a flange (not shown) as a whole. Since the shape of the shell of the noise reduction box 361210 may be a cylinder, the plurality of connecting pipes may be evenly distributed along a circumferential direction of the cylinder. In some embodiments, the plurality of connecting pipes may be unevenly distributed along the circumferential direction of the cylinder. In some embodiments, a gas inlet port may be provided on an outer wall of the support structure 361220, and a gas outlet port may be provided on the shell 361260. In some embodiments, the shape of the shell 361260 may be matched with the shape of the gas pressurization unit 361270, thus the gas pressurization unit 361270 may be tightly buckled in the shell 361260. In some embodiments, as a similar way of the shell of the gas pressurization unit 25190 illustrated in FIG. 25B, the shell 361260 of the gas pressurization unit 361270 may also be made of soft material(s) (e.g., silica gel or soft colloid), so that vibrations generated during the running of the gas pressurization unit 361270 can be absorbed by the soft materials, thereby reducing or eliminating the noise of the gas flowing.

As described above, the noise reduction box 361210 may include three chambers (e.g., the noise reduction box 26205 illustrated in FIGS. 26A and 26B). In some embodiments, the noise reduction box may include two chambers (e.g., the noise reduction box 27370 illustrated in FIGS. 27A and 27B).

In some embodiments, the noise reduction box 361210 may include four chambers. As illustrated in FIG. 36B, the first separation wall 361280 and the second separation wall 361240 may define a first chamber 361241. An inner wall of the upper portion 361211 of the noise reduction box 361210 and the first separation wall 361280 may define a second chamber 361281. The second separation wall 361240 and the third separation wall 361250 may define a third chamber 361251, and the third separation wall 361250 and the inner wall of the lower portion 361212 of the noise reduction box 361210 may define a fourth chamber 361252.

As illustrated in FIG. 36B, a gas may enter the first chamber 361241 via the gas inlet port 361290 of the first chamber 361241 along a direction indicated by the arrow. A connecting pipe 361242 (also referred to as the first pipe) may form a fluid communication between the first chamber 361241 and the second chamber 361281. Thus, after flowing through the first chamber 361241, the gas may be guided to flow from the first chamber 361241 into the second chamber 361281 via the first pipe 361242, for example, along an arrangement direction of the first pipe 361242 indicated by the arrows.

After the gas flows into the second chamber 361281, the gas may further flow into the third chamber 361251 via the connecting pipe 361230 (also refer to as the second pipe), for example, along an arrangement direction of the second pipe 361230 indicated by the arrows. In some embodiments, another connecting pipe 361253 (also referred to as the third pipe) may be disposed between the third chamber 361251 and the fourth chamber 361252. That is, the connecting pipe 361253 may be configured to form a fluid communication between the third chamber 361251 and the fourth chamber 361252. Thus, after the gas flows into the third chamber 361251, the gas may further flow into the fourth chamber 361252 via the connecting pipe 361253. In some embodiments, at least one end of at least one connecting pipe (e.g., the connecting pipe 361230 illustrated in FIG. 36A or 36B) may be beveled, thus a range of a wavelength of noise that may be reduced in the connecting pipe may be widened. The effect of noise reduction of a connecting pipe with a beveled end may be better than the effect of noise reduction of a connecting pipe with non-beveled end. In some embodiments, the at least one end of the at least one connecting pipe may be beveled to facilitate the gas to flow.

In some embodiments, the gas pressurization unit 361270 may be disposed in the fourth chamber 361252, and be in fluid communication with the fourth chamber 361252. A gas inlet port (not shown) of the gas pressurization unit 361270 may be open to or in the fourth chamber 361252, so that the gas entering the fourth chamber 361252 may be capable of being sucked into the gas pressurization unit 361270 via the gas inlet port of the gas pressurization unit 361270. A gas outlet port 361291 of the gas pressurization unit 361270 may be open to an outside of the noise reduction box 361210 to discharge the pressurized gas. In some embodiments, the noise may be generated at the gas inlet port of the gas pressurization unit 361270. The noise may be spread from the third chamber 361251 into the second chamber 361281 via the connecting pipe 361230. Since the second chamber 361281 is larger than the third chamber 361251, the frequency of the noise may be changed with the change of the chamber size. In a similar way, the frequency of the noise may be further changed when the noise is spread from the second chamber 361281 into the first chamber 361241 via the connecting pipe 361242, in which the first chamber 361241 is larger than the second chamber 361281. The frequency change of the noise may reduce the energy of the noise. Therefore, the noise may be reduced when the noise is spread from the first chamber 361241 to the outside of the noise reduction box 361210. Thus, the noise may be reduced effectively. Since the structures of the noise reduction box illustrated in FIG. 30 is in a similar manner with the structures of the noise reduction box 361210, the noise may also be reduced in a similar way in the noise reduction box illustrated in FIG. 30.

In some embodiments, as a similar way of the second chamber 26260 illustrated in FIGS. 26A and 26B, or the second chamber 27350 illustrated in FIG. 27B, the fourth chamber 361252 may also include a guide structure configured to guide the gas to flow into the gas inlet port (not shown) of the gas pressurization unit 361270. In some embodiments, the guide structure may include a conical bulge. The conical bulge may be disposed on an inner wall of the fourth chamber 361252, and may face the gas inlet port of the gas pressurization unit 361270. In some embodiments, the conical bulge may be configured to guide the gas in the fourth chamber 361252 to flow into the gas pressurization unit 361270 via the gas inlet port along a direction indicated by the arrow. The more description of the guide structure may be found elsewhere in the present disclosure, for example, FIG. 30 and relevant descriptions, which may not be repeated herein.

As described above, the first chamber 361241 and the second chamber 361281 may be in fluid communication via the first pipe 361242, the second chamber 361281 and the third chamber 361251 may be in fluid communication via the second pipe 361230, the third chamber 361251 and the fourth chamber 361252 may be in fluid communication via the third pipe 361253, and the fourth chamber 361252 may be in fluid communication with the gas pressurization unit 361270 via the guide structure and the gas inlet port of the gas pressurization unit 361270. That is, each of the four chambers may be in fluid communication with another chamber in a certain order. In some embodiments, each of three of the four chambers may be in fluid communication with another chamber, and the rest of the four chambers may be configured to accommodate the gas pressurization unit 361270, and may not be in fluid communication with other three chambers.

As illustrated in FIG. 36C, the fourth chamber 361252 may be configured to accommodate the gas pressurization unit 361270, and the shell 361260 of the gas pressurization unit 361270 may form a sealed connection with an inner wall of the fourth chamber 361252, and/or fix the gas pressurization unit 361270 to a fixed position (e.g., one or more positions in the fourth chamber 361252, and/or one or more positions on one or more inner walls of the noise reduction box 361210). The fixed position may be determined by a manufacturer of the noise reduction box 361210, which may not be limited in the present disclosure. The third chamber 361251 may not be in fluid communication with the fourth chamber 361252 without a connecting pipe (e.g., a connecting pipe 361253 illustrated in FIG. 36B). That is, the fourth chamber 361252 may be a sealed space separated from other three chambers in the noise reduction box 361210 with the second separation wall 361250. In some embodiments, the gas inlet port (not show) of the gas pressurization unit 361270 may be open to or in the third chamber 361251 and may not be open to or in the fourth chamber 361252. Thus, after the gas flows into the third chamber 361251 from the second chamber 361281, the gas may be sucked into the gas pressurization unit 361270 directly, and the gas outlet port 361291 of the gas pressurization unit 361270 may be still open to an outside of the noise reduction box 361210 to discharge the pressurized gas.

Since the fourth chamber 361252 may be configured to accommodate the gas pressurization unit 361270, the noise generated during the running of the gas pressurization unit 361270 or noise generated by the gas flowing in a position of the gas inlet port of the gas pressurization unit 361270 may be absorbed by the fourth chamber 361252 or the third chamber 361251, thus the noise may be reduced.

It should be noted that the descriptions of the noise reduction box including four chambers are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, a count of the chambers included in the noise reduction box may be greater than four. For example, a count of the chambers included in the noise reduction box may be 5, 6, or 7. In some embodiments, a count of the chambers included in the noise reduction box may be determined based on actual requirements of noise reduction, which may not be limited in the present disclosure.

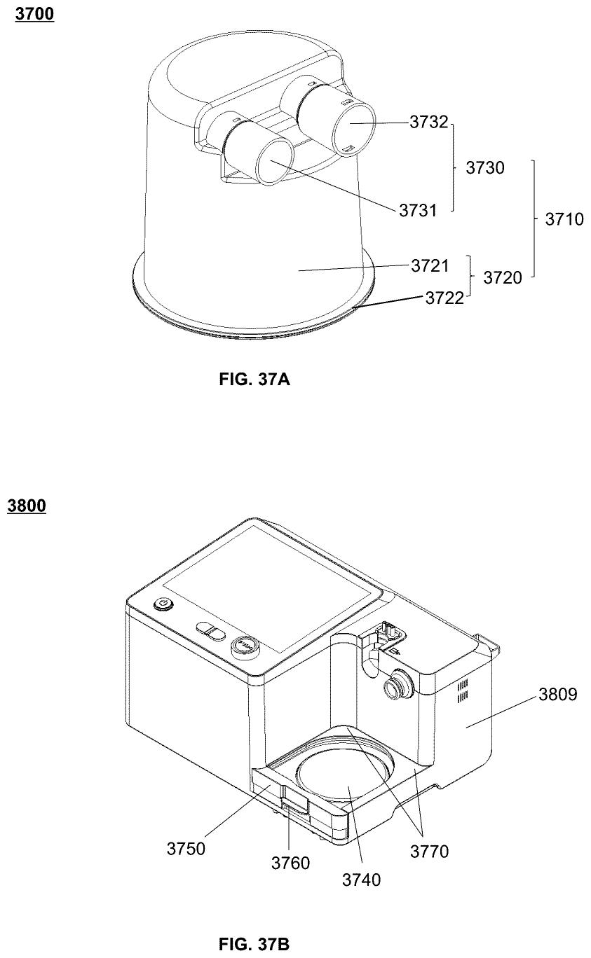

FIG. 37A illustrates an exemplary humidification assembly according to some embodiments of the present disclosure. FIG. 37B illustrates an exemplary ventilatory treatment apparatus without the humidification assembly shown in FIG. 37A according to some embodiments of the present disclosure.

The humidification assembly 3700 may be configured to humidify a gas (e.g., a mixed gas, or a respiratory gas) to provide a humidified respiratory gas for a subject (e.g., the subject 180). Mere by way of example, the subject may include a patient, an experimental subject, a user, etc. In some embodiments, the humidification assembly 3700 may be applied in a ventilatory treatment apparatus (e.g., the ventilatory treatment apparatus 3800 shown in FIG. 37B). In some embodiments, the humidification assembly 3700 may be detachably coupled to the ventilatory treatment apparatus 3800. For example, as shown in FIG. 37B, the main body 3809 of the ventilatory treatment apparatus 3800 may include a heater assembly 3740, a pushing part 3750, and a limiting structure 3770. The pushing part 3750 may be pushed to facilitate the humidification assembly 3700 (e.g., the tank bottom 3722) to slide through. When the pushing part 3750 is pushed down, the tank bottom 3722 may be driven manually to slide through the pushing part 3750, and slide into the prescribed position right above the heater assembly 3740. In some embodiments, a size (e.g., the width, the length, or the diameter) of the tank bottom 3722 may be larger than that of the limiting structure 3770, and then the tank bottom 3722 may be prevented by the limiting structure 3770 from moving up and down, and be limited in a space underneath the limiting structure 3770. After the humidification assembly 3700 slides through the pushing part 3750, and/or after the pressure that pushes down the pushing part 3750 is released, the pushing part 3750 may return back to its initial position under the force of spring return of one or more springs underneath the pushing part 3750. The pushing part 3750 at the initial position may limit the position of the humidification assembly 3700 to prevent the humidification assembly 3700 from falling off the main body 3809, thereby increasing the firmness and stability of the coupling between the humidification assembly 3700 and the main body 3809. The pushing part 3750 may include a pushing plate 3760 configured to facilitate the subject 180 or another person to press the pushing part 3750. For example, the subject or other person(s) may press the pushing plate 3760 to push down the pushing part 3750. More descriptions of the heater assembly 3740 may be found elsewhere in the present disclosure (e.g., FIGS. 55A-55C and descriptions thereof). In some embodiments, the humidification assembly 3700 and the ventilatory treatment apparatus 3800 may be configured as an integral piece. In some embodiments, the humidification assembly 3700 may be coupled to the ventilatory treatment apparatus 3800 in other feasible manners, which is not limited herein.

In some embodiments, as shown in FIG. 37A, the humidification assembly 3700 may include a liquid chamber 3710. The liquid chamber 3710 may be configured to accommodate a liquid (e.g., water). The liquid may be configured to humidify the gas to generate a humidified gas.

In some embodiments, the liquid chamber 3710 may include a tank 3720 and a gas transmission interface 3730. The tank 3720 may include a tank body 3721 and a tank bottom 3722. In some embodiments, the gas transmission interface 3730 may include a gas intake tube 3731 and a gas exit tube 3732. The gas transmission interface 3730 may be configured to facilitate a gas communication between the tank body 3721 with the ventilatory treatment apparatus 3800 (also referred to as an apparatus for ventilatory treatment) and/or the respiration tube 160. For example, the gas communication between the tank body 3721 with the ventilatory treatment apparatus 3800 and/or the respiration tube 160 may be facilitated through the gas intake tube 3731 and the gas exit tube 3732 of the gas transmission interface 3730.

In some embodiments, the tank bottom 3722 may include or be made of one or more metallic heat conducting materials. The metallic heat conducting material(s) may be configured to conduct heat to the liquid. For example, the metallic heat conducting material may include gold, silver, copper, tungsten, zinc, etc. In some embodiments, the tank bottom 3722 may include or be made of one or more non-metallic heat conducting materials. The non-metallic heat conducting material(s) may be configured to conduct heat to the liquid. For example, the non-metallic heat conducting material may include alumina, silicon oxide, zinc oxide, aluminum nitride, boron nitride, silicon carbide, graphite, etc. In some embodiments, the tank bottom 3722 may include a combination of one or more metallic heat conducting materials and one or more non-metallic heat conducting materials. In some embodiments, the tank bottom 3722 may include a metallic heat conducting material layer (made of the metallic heat conducting material(s)), or a non-metallic heat conducting material layer (made of the non-metallic heat conducting material(s)), or the like, or a combination thereof.

In some embodiments, the tank 3720 may be made of the metallic heat conducting material(s) and/or the non-metallic heat conducting material(s). In some embodiments, except the tank bottom 3722, the rest of the tank 3720 may be equipped with an insulation layer configured to prevent or reduce the heat dissipation of the liquid.

In some embodiments, the tank bottom 3722 may be detachably coupled to the tank body 3721. The tank bottom 3722 may be coupled to the tank body 3721 through a snap-in connection, a threaded connection, an adsorption connection, etc. In some embodiments, the tank body 3721 and the tank bottom 3722 may be configured as an integral piece. In some embodiments, the tank bottom 3722 may be coupled to the tank body 3721 through other feasible manners, which is not limited herein.

In some embodiments, the tank bottom 3722 may further include or be equipped with a sealing ring (not shown). The sealing ring may be configured to achieve a sealed connection between the tank body 3721 and the tank bottom 3722. In some embodiments, the tank body 3721 may be wrapped with an edge portion of the tank bottom 3722 without the sealing ring. In some embodiments, the tank body 3721 and the tank bottom 3722 may be in a sealed connection through other feasible manners, which is not limited herein.

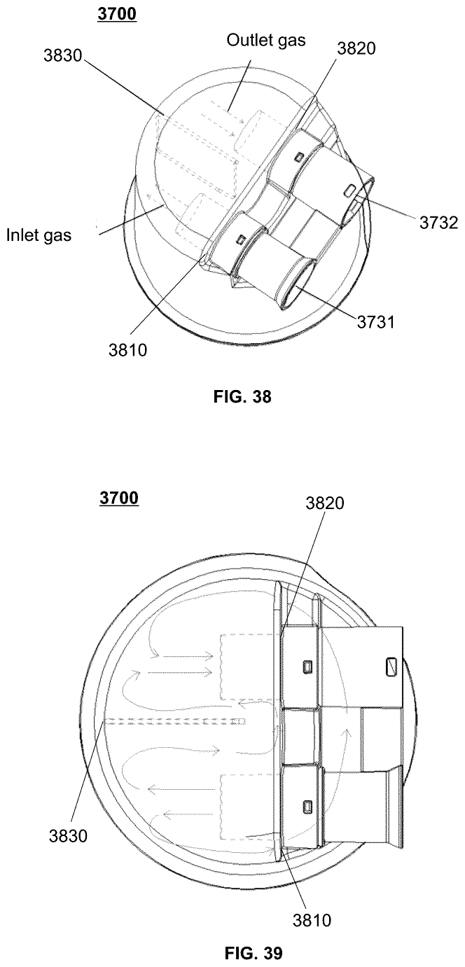

FIG. 38 illustrates an exemplary humidification assembly with a separation board according to some embodiments of the present disclosure.

In some embodiments, the tank body 3721 may include a gas inlet port 3810 and a gas outlet port 3820. The gas inlet port 3810 may be configured to introduce the gas into the tank 3720, and the gas outlet port 3820 may be configured to discharge the humidified gas. For example, the gas inlet port 3810 may introduce the gas into the tank 3720 through the gas intake tube 3731 inserted into the gas inlet port 3810, and the gas outlet port 3810 may discharge the humidified gas through the gas exit tube 3732 inserted into the gas outlet port 3820.

In some embodiments, the gas inlet port 3810 and the gas outlet port 3820 may be disposed on an upper portion of the tank body 3721. The gas inlet port 3810 and the gas outlet port 3820 may be disposed on a same plane. For example, when the liquid is accommodated in the tank 3720, the gas inlet port 3810 and the gas outlet port 3820 may be located above a liquid plane. In some embodiments, a height of the gas inlet port 3810 relative to the tank bottom 3722 may be equal to a height of the gas outlet port 3820 relative to the tank bottom 3722. In some embodiments, the gas inlet port 3810 may be located closer to a left sidewall of the tank 3720 than the gas outlet port 3820 located to a right sidewall of the tank 3720. In some embodiments, the gas inlet port 3810 and the gas outlet port 3820 may be located on two sides of a central axis of the tank body 3721 symmetrically, and the central axis of the tank body 3721 may be perpendicular to the tank bottom 3722. In some embodiments, the gas inlet port 3810 and the gas outlet port 3820 may be located on two sides of a central axis of the tank body 3721 asymmetrically, in which the central axis of the tank body 3721 may be perpendicular to the tank bottom 3722.

In some embodiments, the tank body 3721 may further include a separation board 3830. The separation board 3830 may be configured to increase a length of a flow path of the gas (i.e., a gas flow path) inside the tank body 3721, so that the gas is fully humidified and/or warmed.

In some embodiments, the liquid chamber 3710 may house the separation board 3830. In some embodiments, the separation board 3830 and the tank body 3721 may be configured as an integral piece. In some embodiments, the separation board 3830 may be integrally formed on an inner surface of a top wall of the tank body 3721. For example, the separation board 3830 may extend vertically downward from an inner surface of a top wall of the tank body 3721. In some embodiments, the separation board 3830 may be detachably coupled to the tank body 3721. The separation board 3830 may be coupled to the tank body 3721 in any feasible manner, which is not limited herein.

In some embodiments, the separation board 3830 may be oriented at an angle relative to the plane of the gas inlet port 3810 and the gas outlet port 3820 other than parallel. For example, the separation board 3830 may be oriented at an angle of 90 degrees relative to the plane of the gas inlet port 3810 and the gas outlet port 3820.

In some embodiments, the gas inlet port 3810 and the gas outlet port 3820 may be located on two sides of the separation board 3830. In some embodiments, a distance between the separation board 3830 and the gas inlet port 3810 may be larger than a distance between the separation board 3830 and the gas outlet port 3820, so that the gas can be fully humidified and/or warmed before the gas outlet port 3820 discharges the gas. In some embodiments, a distance between the separation board 3830 and the gas inlet port 3810 may be equal to a distance between the separation board 3830 and the gas outlet port 3820.

In some embodiments, the separation board 3830 may have a plane surface. In some embodiments, the separation board 3830 may have a curved surface. For example, one side of the separation board 3830 may be convex toward a sidewall of the tank body 3721. For example, a side of the separation board 3830 close to the gas outlet port 3820 may be convex toward the right sidewall of the tank body 3721. It should be noted that the left (or right) sidewall refers to the sidewall on the left (or right) side of the tank body 3721 when a subject is facing the plane of the gas inlet port 3810 and the gas outlet port 3820.

In some embodiments, the separation board 3830 may extend from a sidewall of the tank body 3721 toward another sidewall that is opposite to the sidewall of the tank body 3721. For example, the separation board 3830 may extend from a first sidewall of the tank body 3721 to a second sidewall where the plane of the gas inlet port 3810 and the gas outlet port 3820 is located (e.g., between the gas inlet port 3810 and the gas outlet port 3820), and the first sidewall of the tank body 3721 may be opposite to the plane of the gas inlet port 3810 and the gas outlet port 3820. In some embodiments, the separation board 3830 may extend along a radial direction of the liquid chamber 3710. In some embodiments, the separation board 3830 may extend through the liquid chamber 3710. In some embodiments, the separation board 3830 may not extend through the liquid chamber 3710. For example, one or more sides of the separation board 3830 may suspend in the liquid chamber 3710 to let the gas pass through. For instance, a first side of the separation board 3830 away from the plane of the gas inlet port 3810 and the gas outlet port 3820 may extend to (or abut, or form an integral piece with) an inner surface of the first sidewall of the tank body 3721, while a second side of the separation board 3830 (opposite to the first side) may extend (or suspend) between the first sidewall of the tank body 3721 and the second sidewall of the tank body 3721. In some embodiments, a third side of the separation board 3830 close to the top wall of the tank body 3721 may extend to (e.g., abut or from an integral piece with) the inner surface of the top wall of the tank body 3721. In some embodiments, the third side of the separation board 3830 may suspend in the liquid chamber 3710. In some embodiments, a fourth side of the separation board 3830 opposite to the third side of the separation board 3830 may extend toward the tank bottom 3722. For example, the fourth side of the separation board 3830 may suspend in the liquid chamber 3710, so that the gas can flow around the fourth side of the separation board 3830, thereby increasing the gas flow path of the gas in the liquid chamber 3710, making the gas be fully humidified and/or warmed by the liquid. In some embodiments, the separation board 3830 may allow a ratio of the gas directly flowing from the gas inlet port 3810 to the gas outlet port 3820 being less than 30%.

In some embodiments, a width of the separation board 3830 may be smaller than a width of the tank body 3721. The width of the separation board 3830 may refer to a width along a radial direction of the tank body 3721. The radial direction of the tank body 3721 may be parallel to the tank bottom 3722 and/or perpendicular to the plane of the gas inlet port 3810 and the gas outlet port 3820. In some embodiments, the width of the tank body 3721 may be substantially equal to a diameter of the tank bottom 3722. In some embodiments, the width of the separation board 3830 may be a half of the width of the tank body 3721, or a half of the diameter of the tank bottom 3722. In some embodiments, the tank body 3721 may have a cylindrical shape. In some embodiments, the tank body 3721 may have an irregular shape (e.g., a sidewall of the tank body 3721 may be step-shaped), and the width of the separation board 3830 may be a half of a maximum width of the tank body 3721. In some embodiments, the width of the separation board 3830 may be determined based on actual requirements, which is not limited herein.

In some embodiments, a height of the separation board 3830 may be smaller than a height of the tank body 3721. The height of the separation board 3830 may refer to a height along a central axis of the tank body 3721. The central axis of the tank body may be perpendicular to the tank bottom 3722. The height of the tank body may refer to a height from a top wall of the tank body 3721 to the tank bottom 3722. In some embodiments, the separation board 3830 may be separated from the tank bottom 3722. For example, when the liquid is accommodated in the tank 3720, the separation board 3830 may be separated from the liquid plane. As another example, when the liquid is accommodated in the tank 3720, a lower portion of the separation board 3830 may be immersed into the liquid and separated from the tank bottom 3722.

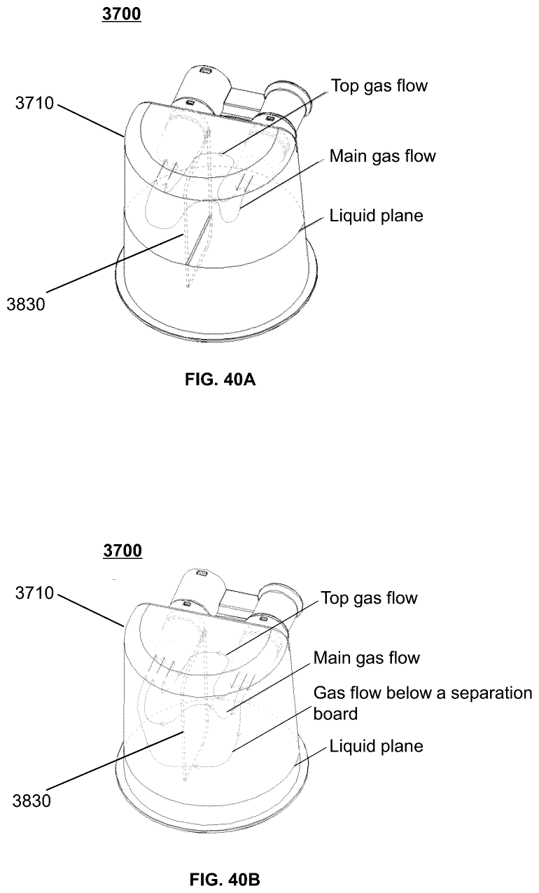

FIG. 39 illustrates a gas flow of an exemplary humidification assembly with a separation board from a top view according to some embodiments of the present disclosure. FIG. 40A illustrates a gas flow of an exemplary humidification assembly with a separation board from a side view when the separation board is lower than a liquid plane according to some embodiments of the present disclosure. FIG. 40B illustrates a gas flow of an exemplary humidification assembly with a separation board from a side view when the separation board is higher than a liquid plane according to some embodiments of the present disclosure.

In some embodiments, the separation board 3830 may be positioned such that a gas flow in the liquid chamber 3710 may be separated into a plurality of portions, thus, the gas entering the tank body 3721 via the gas inlet port 3810 may fully contact with the liquid in the liquid chamber 3710, and be fully humidified and/or warmed by the liquid, and the humidified gas may flow away from the tank body 3721 through the gas outlet port 3820.

In some embodiments, as shown in FIG. 39, the separation board 3830 may be positioned such that at least a first portion of the gas entering the tank body 3721 via the gas inlet port 3810 may arrive at a first side of the separation board 3830, flow in a direction parallel to the tank bottom 3722 (e.g., the main gas flow shown in FIGS. 40A and 40B), along the separation board 3830, and around the separation board 3830 to a second side of the separation board 3830, and then flow out of the tank body 3721 through the gas outlet port 3820. In some embodiments, as shown in FIG. 39, another portion of the gas entering the tank body 3721 via the gas inlet port 3810 may flow to (or arrive at) the left sidewall of the tank body 3721, flow along the left sidewall and the front sidewall of the tank body 3721 to the right sidewall of the tank body 3721, then the portion of the gas may flow out of the tank body 3721 through the gas outlet port 3820.

In some embodiments, as shown in FIGS. 40A and 40B, the separation board 3830 may be positioned such that at least a second portion of the gas entering the tank body 3721 via the gas inlet port 3810 may arrive at the first side of the separation board 3830, flow in a direction perpendicular to the tank bottom 3722 (e.g., the top gas flow and gas flow below the separation board 3830 shown in FIGS. 40A and 40B), along the separation board 3830 and around the separation board 3830 to a second side of the separation board 3830, and then flow out of the tank body 3721 through the gas outlet port 3820.

In some embodiments, as shown in FIG. 40A, when a liquid plane is higher than the lower portion of the separation board 3830, the gas flow(s) in the liquid chamber 3710 may include the top gas flow and the main gas flow. The top gas flow may refer that at least the second portion of the gas entering the tank body 3721 via the gas inlet port 3810 may arrive at the first side of the separation board 3830, flow, in the direction perpendicular to the tank bottom 3722 toward the top wall of the tank body 3721, along the separation board 3830 and around the separation board 3830 to the second side of the separation board 3830, and then flow out of the tank body 3721 through the gas outlet port 3820. Details about the main gas flow may refer to the descriptions about the position of the separation board 3830 mentioned above, which is not repeated herein.

In some embodiments, as shown in FIG. 40B, when the separation board 3830 is separated from the liquid plane (or the liquid plane is lower than the lower portion of the separation board 3830), a gas flow in the liquid chamber 3710 may include the top gas flow, the main gas flow, and the gas flow below the separation board 3830. The gas flow below the separation board 3830 may refer that at least a third portion of the gas entering the tank body 3721 via the gas inlet port 3810 may arrive at the first side of the separation board 3830, flow, in the direction perpendicular to the tank bottom 3722 toward the tank bottom 3722, along the separation board 3830 and around the separation board 3830 to the second side of the separation board 3830, and then flow out of the tank body 3721 through the gas outlet port 3820. Details about the main gas flow and the top gas flow may refer to the descriptions about the position of the separation board 3830 mentioned above, which is not repeated herein.

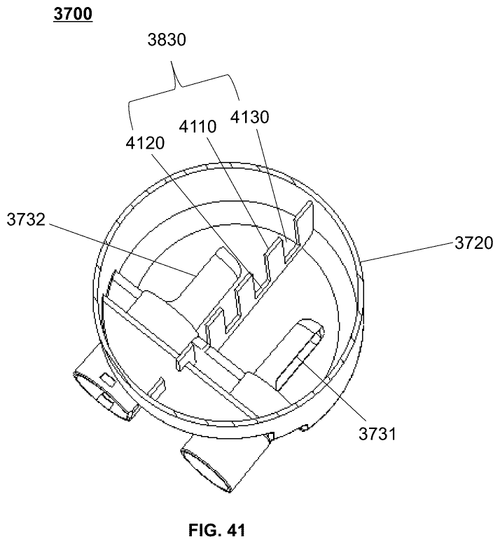

FIG. 41 illustrates an exemplary separation board including a plurality of blocking portions according to some embodiments of the present disclosure.

In some embodiments, as shown in FIG. 41, the separation board 3830 may include a plurality of blocking portions 4110 and a base portion 4120. In some embodiments, the plurality of blocking portions 4110 may be configured as an integral piece, and connected to the inner surface of the top wall of the tank body 3721 through the base portion 4120. A plurality of gas flow channels 4130 may be formed between any two adjacent blocking portions 4110 of the plurality of blocking portions 4110.

In some embodiments, the plurality of blocking portions 4110 may be separated from each other. For example, each of the plurality of blocking portions 4110 may be connected to the inner surface of the top wall of the tank body 3721, extend from the inner surface of the top wall of the tank body 3721 to the tank bottom 3722, and be separated from the tank bottom 3722.

In some embodiments, a width of each of the plurality of blocking portions 4110 may be of a same size, and accordingly, a width of each of the plurality of gas flow channels 4130 may be of a same size. In some embodiments, widths of different blocking portions 4110 may be of different sizes, and accordingly, widths of different gas flow channels 4130 may be of different sizes. In some embodiments, a width of the blocking portion 4110 and a width of the gas flow channel 4130 may be of a same size or different sizes.

In some embodiments, a height of each of the plurality of blocking portions 4110 may be of a same size. The height of a blocking portion 4110 may be a height extending from the inner surface of the top wall of the tank body 3721 or the base portion to the end of the blocking portion 4110, and the blocking portion 4110 may be separated from the tank bottom 3722. In some embodiments, a height of different blocking portions 4110 may be of different sizes, and a depth of the plurality of gas flow channels 4130 may be of different sizes.

In some embodiments, a portion of the gas in the tank 3720 may pass through the plurality of gas flow channels 4130 and flow to the gas exit tube 3732. Another portion of the gas in the tank 3720 may be blocked by the plurality of blocking portions 4110 and flow downward, so as to improve a humidification effect of the gas with the liquid in the tank 3720 and reduce a resistance of the gas flowing from the gas intake tube 3731 to the gas exit tube 3732. Merely by way of example, the humidified gas in the tank 3720 may flow upward along two sides of each of the plurality of blocking portions 4110, and the gas flow may be mixed with the humidified gas at a contact surface between the blocking portion(s) 4110 and the gas flow channel(s), so that the gas flow in a dry and cold state may be changed to a mixed gas flow in a humidified state, thereby improving a humidification effect of the gas flow and mixing efficiency.

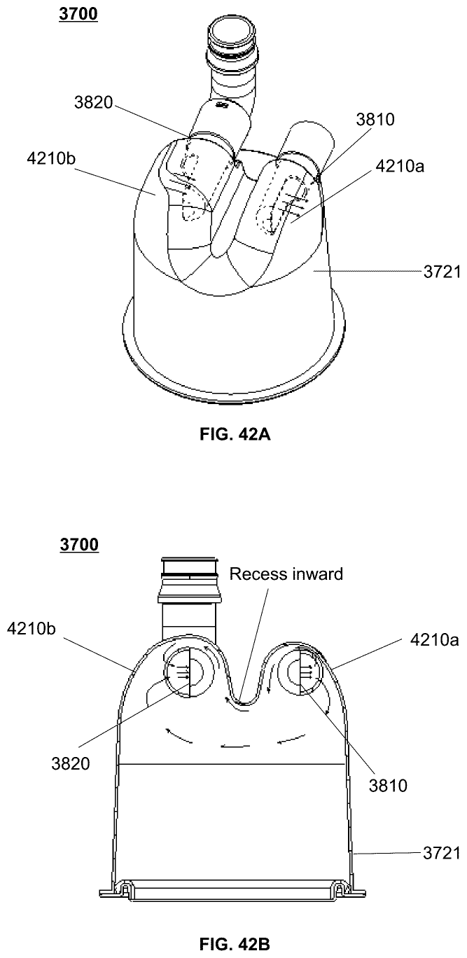

FIGS. 42A and 42B illustrate an exemplary tank without a separation board according to some embodiments of the present disclosure.

In some embodiments, as shown in FIGS. 42A and 42B, the separation board 3830 may be omitted. In some embodiments, an upper portion of the liquid chamber 3710 may include a first convex chamber 4210a and a second convex chamber 4210b. The first convex chamber 4210a may be configured to accommodate the gas inlet port 3810, and the second convex chamber 4210b may be configured to accommodate the gas outlet port 3820. For example, a left portion of the top wall of the tank body 3721 may be convex toward outside of the tank body 3721 to form the first convex chamber 4210a, and a right portion of the top wall of the tank body 3721 may be convex toward outside of the tank body 3721 to form the second convex chamber 4210b. It should be noted that the left (or right) portion refers to the top wall on the left (or right) side of the tank body 3721 when a subject is facing the plane of the gas inlet port 3810 and the gas outlet port 3820. The left portion may be adjacent to the gas intake tube 3731, while the right portion may be adjacent to the gas exit tube 3732. In some embodiments, the first convex chamber 4210a and the second convex chamber 4210b may be symmetrically located on two sides of the central axis of the tank body 3721 that is perpendicular to the tank bottom 3722. Alternatively, in some embodiments, the first convex chamber 4210a and the second convex chamber 4210b may be asymmetrically located on two sides of the central axis of the tank body 3721 that is perpendicular to the tank bottom 3722. As another example, a portion of the top wall of the tank body 3721 may be recessed inward, and two sides of the recessed portion of the top wall of the tank body 3721 may be configured as the first convex chamber 4210a and the second convex chamber 4210b, respectively. In some embodiments, the recessed portion of the top wall of the tank body 3721 may be positioned at the central axis of the tank body 3721 that is perpendicular to the tank bottom 3722. In some embodiments, the recessed portion of the top wall of the tank body 3721 may be positioned offset from the central axis of the tank body 3721 that is perpendicular to the tank bottom 3722.

In some embodiments, as shown in FIG. 42B, at least a portion of the gas entering the tank body 3721 via the gas inlet port 3810 may arrive at a first side of the recessed portion of the top wall of the tank body 3721, flow, in a direction perpendicular to the tank bottom 3722, along the recessed portion and around the recessed portion to a second side of the recessed portion, and then flow out of the tank body 3721 through the gas outlet port 3820.

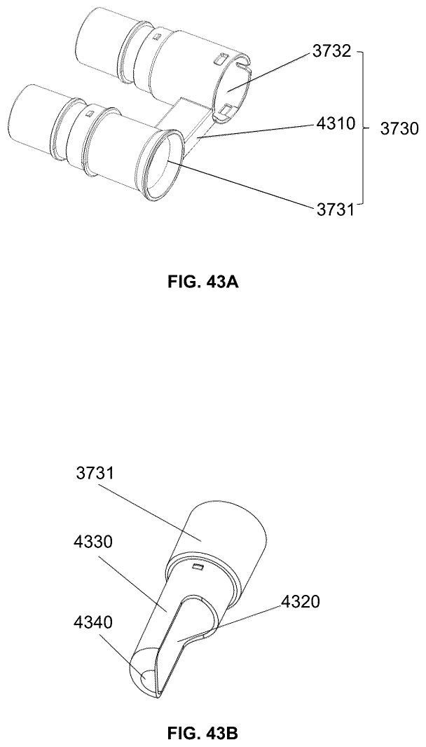

FIG. 43A illustrates an exemplary gas transmission interface according to some embodiments of the present disclosure. FIG. 43B illustrates an exemplary gas intake tube according to some embodiments of the present disclosure.

In some embodiments, the gas transmission interface 3730 may include a gas intake tube 3731 and a gas exit tube 3732. The gas intake tube 3731 may be configured to guide the gas from the apparatus to flow into the tank body 3721, and the gas exit tube 3732 may be configured to guide the humidified gas to flow out of the tank body 3721 and into the respiration tube. In some embodiments, the gas intake tube 3731 and the gas exit tube 3732 may extend through the tank body 3721 and enter into the liquid chamber 3710, respectively, so that the gas intake tube 3731 and the gas exit tube 3732 may be in communication with the liquid chamber 3710.

In some embodiments, the gas intake tube 3731 and the gas exit tube 3732 may extend through the tank body 3721 and enter into the liquid chamber 3710 along a horizontal direction. Ends of the gas intake tube 3731 and the gas exit tube 3732 entering into the liquid chamber 3710 may separate from a sidewall along a circumferential direction of the tank body 3721. In some embodiments, an upper part of the tank body 3721 may be step-shaped (see FIG. 37A). The gas intake tube 3731 and the gas exit tube 3732 may be disposed in the upper part and extend into the tank body 3721. In the cases mentioned above, the gas intake tube 3731 and the gas exit tube 3732 may not occupy additional space corresponding to the height of the tank body 3721, thereby avoiding possible instability caused by an excessive height of the tank body 3721. In some embodiments, the end(s) of the gas intake tube 3731 (and/or the gas exit tube 3732) entering into the liquid chamber 3710 may be bent toward the tank bottom 3722 and separate from the tank bottom 3722. In some embodiments, the end(s) of the gas intake tube 3731 (and/or the gas exit tube 3732) entering into the liquid chamber 3710 may be bent toward the top wall of the tank body 3721 and separate from the top wall.

In some embodiments, the gas intake tube 3731 and/or the gas exit tube 3732 may be hollow cylindrical tubes (see FIG. 43A). In some embodiments, the gas intake tube 3731 and the gas exit tube 3732 may be of other shapes, such as hollow prismatic tubes.

In some embodiments, end(s) of the gas intake tube 3731 and/or the gas exit tube 3732 entering into the liquid chamber 3710 may include one or more notches of a hollow semi-cylindrical structure, respectively. As shown in FIG. 43B, taking the gas intake tube 3731 as an example, the end of the gas intake tube 3731 entering into the liquid chamber 3710 may include a notch 4320, a sidewall 4330 along a circumferential direction, and an end wall 4340. The notch 4320, the sidewall 4330, and the end wall 4340 may form the hollow semi-cylindrical structure to communicate with the liquid chamber 3710. The hollow semi-cylindrical structure may facilitate a fully contact of the gas and the liquid in the tank body 3721, thereby allowing the gas to be sufficiently humidified when the gas flows in the tank body 3721. In some embodiments, the liquid included in the humidified gas may be no less than 33 mg.

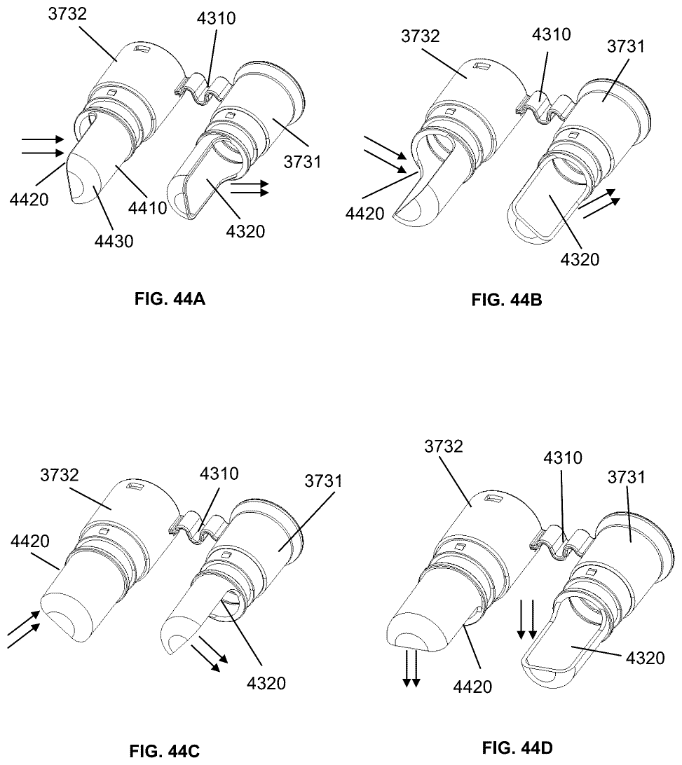

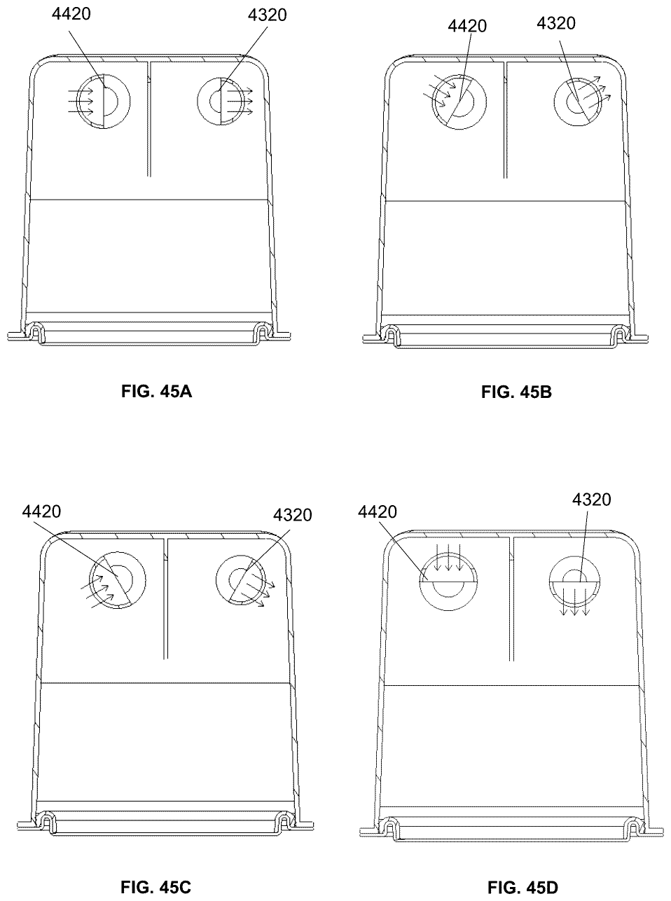

FIGS. 44A-45D illustrate exemplary opening directions of a notch of a gas intake tube and a notch of a gas exit tube according to some embodiments of the present disclosure.

In some embodiments, as shown in FIGS. 44A-45D, the end of the gas exit tube 3732 entering the liquid chamber 3710 may have a (substantially) identical structure with the end of the gas intake tube 3731 entering the liquid chamber 3710. As used herein, substantially, when used to qualify a feature (e.g., equivalent to, the same as, etc.), indicates that the deviation from the feature is below a threshold, e.g., 30%, 25%, 20%, 15%, 10%, 5%, etc. In some embodiments, the end of the gas exit tube 3732 may include a sidewall 4410 along a circumferential direction, a notch 4420, and an end wall 4430. The sidewall 4410, the notch 4420, and the end wall 4430 may form a hollow semi-cylindrical structure to communicate with the liquid chamber 3710.

In some embodiments, the gas may flow into the tank body 3721 through the notch 4320 of the gas intake tube 3731, and flow out of the tank body 3721 through a notch 4420 of the gas exit tube 3732. In some embodiments, opening directions of the notch 4320 and the notch 4420 may have an impact on the gas flow in the tank body 3721. In some embodiments, an opening direction of the notch 4320 may deviate from an opening direction of the notch 4420. For example, the opening direction of the notch 4320 may face away from the gas exit tube 3732, and the opening direction of the notch 4420 may face away from the gas intake tube 3731. In some embodiments, the gas flowing into the tank body 3721 through the notch 4320 may flow to a side (e.g., the left sidewall of the tank body 3721) away from the gas exit tube 3732, and flow into the gas exit tube 3732 along a side (e.g., the right sidewall of the tank body 3721) away from the gas intake tube 3731, thereby extending the gas flow path in the tank body 3721 and ensuring a fully contact with the liquid in the liquid chamber 3710 to humidify and/or warm the gas.

In some embodiments, as shown in FIG. 44A and FIG. 45A, the opening direction of the notch 4320 and the opening direction of the notch 4420 may be substantially identical to a horizontal direction and/or may be symmetrical to each other. The gas may flow into the tank body 3721 along the horizontal direction through the notch 4320, and flow downward along the sidewall (adjacent to the gas intake tube 3731) of the tank body 3721. The gas may further flow upward along the sidewall (adjacent to the gas exit tube 3732) of the tank body 3721, and flow into the gas exit tube 3732 along the horizontal direction through the notch 4420.

In some embodiments, as shown in FIG. 44B and FIG. 45B, the opening direction of the notch 4320 and the opening direction of the notch 4420 may be inclined with respect to the horizontal direction and may not be completely symmetrical to each other. The opening direction of the notch 4320 may deviate from the opening direction of the notch 4420. In some embodiments, the opening direction(s) of the notch 4320 and/or the notch 4420 may be in an inclined upward direction relative to the horizontal direction. For example, as shown in FIG. 44B and FIG. 45B, the gas may flow into the tank body 3721 along an inclined upward direction relative to the horizontal direction through the notch 4320, and flow downward along the sidewall (adjacent to the gas intake tube 3731) of the tank body 3721. The gas may further flow upward along the sidewall (adjacent to the gas exit tube 3732) of the tank body 3721, and flow into the gas exit tube 3732 along an inclined downward direction relative to the horizontal direction through the notch 4420.

In some embodiments, as shown in FIG. 44C and FIG. 45C, the opening direction(s) of the notch 4320 and/or the notch 4420 may be in an inclined downward direction relative to the horizontal direction. For example, as shown in FIG. 44C and FIG. 45C, the gas may flow into the tank body 3721 along an inclined downward direction relative to the horizontal direction through the notch 4320, and flow downward along the sidewall (adjacent to the gas intake tube 3731) of the tank body 3721. The gas may further flow upward along the sidewall (adjacent to the gas exit tube 3732) of the tank body 3721, and flow into the gas exit tube 3732 along an inclined upward direction relative to the horizontal direction through the notch 4420.

In some embodiments, as shown in FIG. 44D and FIG. 45D, the opening directions of the notch 4320 and the notch 4420 may be opposite. For example, as illustrated in FIG. 45D, the opening of the notch 4320 may face downward along a vertical direction, and the opening of the notch 4420 may face upward along the vertical direction. The gas may flow into the tank body 3721 along the vertical direction through the notch 4320, and flow into the gas exit tube 3732 along the vertical direction through the notch 4420. In some embodiments, as illustrated in FIG. 44D, the opening of the notch 4320 may face upward along a vertical direction, and the opening of the notch 4420 may face downward along the vertical direction. As shown in FIGS. 44A-45D, opening directions of the notch 4320 and the notch 4420 may direct the gas flowing to the sidewalls and inside of the tank body 3721, thereby avoiding the gas flowing out of the tank body 3721 through the gas exit tube 3732 directly, and ensuring a humidification effect of the gas.

In some embodiments, the gas intake tube 3731 and the gas exit tube 3732 may be detachably coupled to the tank body 3721, the apparatus, and the respiration tube. For example, the gas intake tube 3731 and the gas exit tube 3732 may be inserted into the gas inlet port 3810 and the gas outlet port 3820 located on the tank body 3721 through a snap-in connection, a threaded connection, etc., respectively. More details about the gas inlet port 3810 and the gas outlet port 3820 may refer to FIG. 38 and related descriptions thereof. In some embodiments, the gas intake tube 3731, the gas exit tube 3732, the tank body 3721, or the apparatus may be configured as an integral piece. In some embodiments, the gas intake tube 3731 and the gas exit tube 3732 may be coupled to the tank body 3721, the apparatus, and the respiration tube through other feasible manners, which is not limited herein.

In some embodiments, as shown in FIGS. 43A and 44A-44D, the gas transmission interface 3730 may further include a connecting rib 4310. The connecting rib 4310 may be configured to connect the gas intake tube 3731 and the gas exit tube 3732, thereby avoiding the gas intake tube 3731 and the gas exit tube 3732 to rotate in the gas inlet port 3810 and the gas outlet port 3820.

In some embodiments, the gas intake tube 3731, the gas exit tube 3732, and the connecting rib 4310 may be configured as an integral piece. In some embodiments, the gas transmission interface 3730 may be assembled using the gas intake tube 3731, the gas exit tube 3732, and the connecting rib 4310.

In some embodiments, as shown in FIG. 43A, the connecting rib 4310 may be of a one-piece shape. In some embodiments, as shown in FIGS. 44A-44D, the connecting rib 4310 may be of a wave shape. In some embodiments, the connecting rib 4310 may be of any other shape, which is not limited herein.

In some embodiments, the gas intake tube 3731 and/or the gas exit tube 3732 may include one or more holes (not shown). The one or more holes may be disposed close to the end(s) of the gas intake tube 3731 and/or the gas exit tube 3732 that are inserted into the tank body 3721. In some embodiments, the one or more holes may open to the top wall of the tank body 3721. In some embodiments, the one or more holes may be configured to facilitate at least a portion of the gas flowing from the gas intake tube 3731 to flow to an upper wall (or the top wall) of the liquid chamber 3710 to drive at least a portion of the gas in an upper portion (e.g., the gas residing above the gas inlet port 3810 and the gas outlet port 3820) of the liquid chamber 3710 to flow to a bottom portion of the liquid chamber 3710, so that the at least a portion of the gas may be completely mixed with the gas in the tank body 3721, thereby improving the a humidification effect of the gas. In some embodiments, the one or more holes may be configured to facilitate at least a portion of the humidified gas in the upper portion of the liquid chamber 3710 to flow into the gas exit tube 3732.

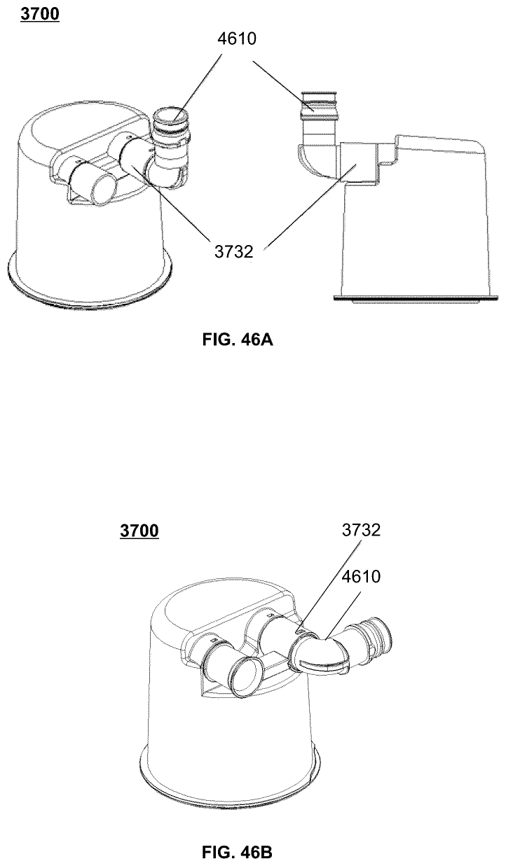

FIGS. 46A and 46B illustrate an exemplary first connecting tube according to some embodiments of the present disclosure.

In some embodiments, as shown in FIGS. 46A and 46B, the humidification assembly 3700 may further include a first connecting tube 4610. The first connecting tube 4610 may be configured to connect the gas exit tube 3732 with the respiration tube. The first connecting tube 4610 may be in fluid communication with the tank body 3721 through the gas exit tube 3732, so as to provide the humidified gas to the subject.

In some embodiments, the first connecting tube 4610 may include a horizontal extending segment coupled to the gas exit tube 3732, and a vertical extending segment coupled to the respiration tube. In some embodiments, as shown in FIG. 46A, the first connecting tube 4610 may be bent upward along a vertical direction relative to an extending direction of the gas exit tube 3732, so as to be connected to the respiration tube along a suitable angle to provide the humidified gas to the subject. In some embodiments, as shown in FIG. 46B, the first connecting tube 4610 may be bent perpendicular to the extending direction of the gas exit tube 3732 in a horizontal plane, so as to be connected to the respiration tube along another suitable angle to provide the humidified gas to the subject. In some embodiments, the first connecting tube 4610 may be bent toward other directions, and a bending angle between the first connecting tube 4610 and the extending direction of the gas exit tube 3732 may be other angles that are suitable for a connection between the first connecting tube 4610 and the respiration tube, which is not limited herein.

FIGS. 47A and 47B illustrate an exemplary electrical interface of the ventilatory treatment apparatus according to some embodiments of the present disclosure. FIG. 47A shows the electrical interface and the first connecting tube from a side view. FIG. 47B shows the electrical interface and the first connecting tube from a top view.

In some embodiments, the first connecting tube 4610 may be detachably coupled to an apparatus (e.g., the main body 209 of the ventilatory treatment apparatus 200). The first connecting tube 4610 may be coupled to the apparatus through a snap-in connection, an electrical connection, an adsorption connection, etc. In some embodiments, the first connecting tube 4610 may be coupled to the apparatus through a fixed connection, such as a welding connection, etc. In some embodiments, the first connecting tube 4610 may be coupled to the apparatus through other feasible manners, which is not limited herein.

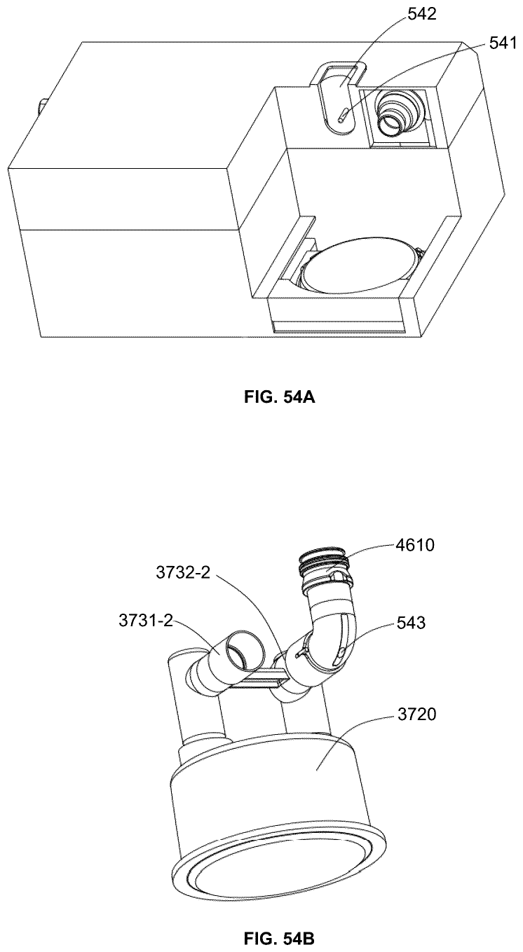

In some embodiments, the apparatus may include a positioning slot (see the positioning slot 542 in FIG. 54A). The positioning slot may be configured to locate the first connecting tube 4610. In some embodiments, the positioning slot may include a limiting slot. An end of the first connecting tube 4610 coupled to the apparatus may include a positioning hook. The positioning hook may be matched with the limiting slot included in the positioning slot to restrict the detachment of the first connecting tube 4610 from the apparatus.

In some embodiments, the positioning hook may be bent upward to be hooked into the limiting slot. In some embodiments, the first connecting tube 4610 may be hooked into the limiting slot automatically through an elastic mechanism. For example, an elastic component (e.g., a spring, etc.) may be arranged in a supporting base (e.g., a heating plate (also referred to as a heat transfer plate) used to heat the liquid in the tank 3720 of the apparatus), the supporting base may be used to support the tank 3720, and the tank 3720 may be pushed upward until the positioning hook of the first connecting tube 4610 is hooked into the limiting slot. In some embodiments, a hooked length between the positioning hook and the limiting slot may be in a range of 0.5 mm-5 mm. For example, the hooked length between the positioning hook and the limiting slot may be 1 mm.

In some embodiments, the positioning hook may be fitted with the limiting slot to restrict the detachment of the tank 3720 from the apparatus along a horizontal direction. In some embodiments, the limiting slot may restrict the movement of the first connecting tube 4610 in other directions, which is not limited herein.

In some embodiments, the first connecting tube 4610 may be disposed adjacent to an electrical interface 4710 of the apparatus. The electrical interface 4710 may be configured to provide an electrical connection between the apparatus and the respiration tube, such that the respiration tube may be capable of being coupled to the first connecting tube 4610 and the electrical interface 4710 simultaneously.

In some embodiments, the positioning slot may be disposed adjacent to the electrical interface 4710. When the tank 3720 is mounted on the apparatus, the first connecting tube 4610 may be positioned into the positioning slot, and the respiration tube may be coupled to the first connecting tube 4610.

FIGS. 54A-54B illustrate an exemplary temperature measurement sensor according to some embodiments of the present disclosure. In some embodiments, as shown in FIG. 54B, the gas inlet port and the gas outlet port of the tank body may be disposed on the top wall of the tank body, and accordingly, the gas intake tube 3731-2 and the gas exit tube 3732-2 may be inserted into the gas inlet port and the gas outlet port vertically. In some embodiments, as shown in FIG. 54B, the gas intake tube 3731-2 and the gas exit tube 3732-2 may have corresponding bending parts, and the bending parts can be inserted into the gas inlet port and the gas outlet port vertically. In some embodiments, the apparatus may include a temperature measurement sensor (see the temperature measurement sensor 541 in FIG. 54A). The temperature measurement sensor 541 may be configured to measure the temperature of the humidified gas in the tank body 3721. In some embodiments, the temperature measurement sensor 541 may protrude relative to an outer contour of the apparatus.

In some embodiments, the tank 3720 may include a temperature measurement chamber 543. The temperature measurement chamber 543 may be configured to accommodate the temperature measurement sensor 541. For example, when the tank 3720 is mounted on the apparatus, the temperature measurement sensor 541 may be accommodated in the temperature measurement chamber 543. In some embodiments, the temperature measurement sensor 541 may be protruded and extended into the temperature measurement chamber 543 to measure the temperature of the humidified gas in the tank body 3721 accurately, thereby providing reliable data for controlling the temperature and the humidity of the gas in the tank body 3721. In some embodiments, when the tank 3720 is separated from the apparatus, the temperature measurement sensor 541 may exit from the temperature measurement chamber 543. The temperature measurement chamber 543 may be configured to be isolated from a gas channel of the first connecting tube 4610. For example, an end of the temperature measurement chamber 543 facing the gas channel may be sealed, and the temperature measurement sensor 541 may be regularly disinfected to save costs.

In some embodiments, the temperature measurement sensor 541 and the temperature measurement chamber 543 may be disposed adjacent to the gas exit tube 3732. In some embodiments, the temperature measurement chamber 543 may be disposed on the first connecting tube 4610. For example, the temperature measurement chamber 543 may be disposed on a sidewall of the first connecting tube 4610 along a circumferential direction, so that the temperature of the humidified gas provided to the subject through the respiration tube coupled to the first connecting tube 4610 may be measured accurately.

In some embodiments, the temperature measurement sensor 541 may be disposed in the positioning slot 542 and extend along a horizontal direction perpendicular to a plane where the positioning slot 542 is located. When the tank 3720 is mounted on the apparatus, the temperature measurement sensor 541 may be inserted into the temperature measurement chamber 543 disposed on the sidewall of the first connecting tube 4610. In some embodiments, the temperature measurement chamber 543 may be disposed on a bottom wall of the horizontal extending segment of the first connecting tube 4610 (see FIG. 46B), so as to provide an enough space for the temperature measurement sensor 541.

In some embodiments, the temperature measurement sensor 541 may be coupled to the apparatus through various feasible manners. For example, the temperature measurement sensor 541 may be pressed and fixed on the apparatus through a pressing plate and a threaded fastener. As another example, the temperature measurement sensor 541 may be fixed on the apparatus through a gluing connection, a snap-in connection, etc.

In some embodiments, the temperature measurement sensor 541 may be in a shape of slender rod. In some embodiments, an extending length of the temperature measurement sensor 541 inserted into the temperature measurement chamber 543 may be in a range of 8 mm-12 mm. For example, the extending length of the temperature measurement sensor 541 inserted into the temperature measurement chamber 543 may be 10 mm.

In some embodiments, an end of the temperature measurement sensor 541 facing the first connecting tube 4610 may include a sensor element, and the other end of the temperature measurement sensor 541 away from the first connecting tube 4610 may be connected to two wires. For example, the end of the temperature measurement sensor 541 facing the first connecting tube 4610 may be a sphere with a diameter of 1 mm.

In some embodiments, the outside of the two wires may be covered with glue. An outer contour of the glue covered on the two wires may be in a shape of cylinder, prism, etc., to improve a sealing performance. In some embodiments, a size of an outer contour of the temperature measurement sensor 541 may be (substantially) equivalent to a size of an inner contour of the temperature measurement chamber 543. As used herein, substantially, when used to qualify a feature (e.g., equivalent to, the same as, etc.), indicates that the deviation from the feature is below a threshold, e.g., 30%, 25%, 20%, 15%, 10%, 5%, etc. For example, when the outer contour of the temperature measurement sensor 541 is in a shape of cylinder, an outer diameter of the temperature measurement sensor 541 may be slightly less than an inner diameter of the temperature measurement chamber 543.

In some embodiments, when the temperature measurement sensor 541 is inserted into the temperature measurement chamber 543, portions of the temperature measurement sensor 541 covered with glue may be in a sealing contact with an inner wall of the temperature measurement chamber 543, and the sensor element may be sealed in the temperature measurement chamber 543 to improve a sealing performance.

In some embodiments, the humidification assembly 3700 may further include a second connecting tube. The second connecting tube may be configured to connect a gas filter unit to the gas intake tube 3731. In some embodiments, the gas filter unit may be directly connected to the gas intake tube 3731 (i.e., the second connecting tube may be deleted). The gas filter unit may be configured to filter the gas entering the liquid chamber 3710.

In some embodiments, the gas filter unit may be disposed on an extending direction of the second connecting tube. The gas flowing into the gas intake tube 3731 may be filtered through the gas filter unit. For example, the gas filter unit may be disposed between the gas intake tube 3731 and the second connecting tube. In some embodiments, the second connecting tube may include an extending segment coupled to the gas intake tube 3731 along a horizontal direction, and the gas filter unit may be disposed on the extending segment of the second connecting tube. In some embodiments, the second connecting tube may include a free end, and the gas filter unit may be coupled to the free end of the second connecting tube. The gas intake tube 3731 may be coupled to the gas filter unit and the second connecting tube in sequence. In some embodiments, one of an end of the gas filter unit and an end of the second connecting tube may include an inner cone connector, the other one of the end of the gas filter unit and the end of the second connecting tube may include an outer cone connector, and the inner cone connector and the outer cone connector may be fitted with each other to achieve a sealed connection. For example, an end of the second connecting tube facing the gas filter unit may include an inner cone surface, and an end of the gas filter unit may include an outer cone surface that is matched with the inner cone surface, so that the outer cone surface may be inserted into the inner cone surface of the second connecting tube to achieve a sealed connection, thereby ensuring a connection reliability and an air tightness. In some embodiments, a diameter of the second connecting tube may be 22 mm.

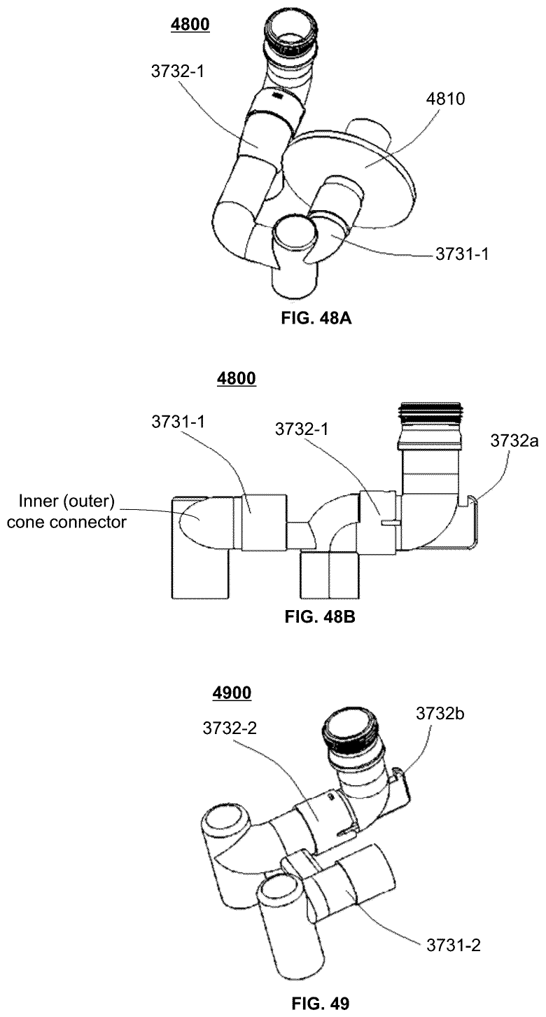

FIGS. 48A and 48B illustrate an exemplary connecting adapter according to some embodiments of the present disclosure.

In some embodiments, as shown in FIG. 48A, a gas intake tube 3731-1 and a gas exit tube 3732-1 may be configured as an integral piece to form a first connecting adapter 4800. The first connecting adapter 4800 may be connected with a gas filter unit 4810. In some embodiments, the first connecting adapter 4800 may be separated from the tank 3720. In some embodiments, the first connecting adapter 4800 and the tank 3720 may be configured as an integral piece. In some embodiments, the first connecting adapter 4800 may be coupled to the tank 3720 in other feasible manners, which is not limited herein.

In some embodiments, the gas filter unit 4810 may be disposed between the gas intake tube 3731-1 and a gas outlet port of the ventilatory treatment apparatus 3800. The gas filter unit 4810 may be coupled to the gas outlet port of the main body of the ventilatory treatment apparatus 200. In some embodiments, the gas filter unit 4810 may be disposed on an extending direction of the gas intake tube 3731-1. In some embodiments, the tank 3720 may be rotated relative to the ventilatory treatment apparatus 200, so as to increase a distance between the tank 3720 and the ventilatory treatment apparatus 200, thereby disposing the gas filter unit 4810 between the gas intake tube 3731-1 and the gas outlet port of the main body of the ventilatory treatment apparatus 200.

In some embodiments, the gas intake tube 3731-1 may include an extending section facing the gas outlet port of the main body of the ventilatory treatment apparatus 200, so as to be connected to the gas outlet port of the main body of the ventilatory treatment apparatus 200.

In some embodiments, the tank 3720 may include a gas inlet port and a gas outlet port that are matched to the gas intake tube 3731-1 and the gas exit tube 3732-1 of the first connecting adapter 4800. For example, the gas inlet port and the gas outlet port may be disposed on the top of the tank 3720. In some embodiments, an angle between a horizontal connection line of the gas inlet port and the gas outlet port and an extending section of the gas intake tube 3731-1 and/or the gas exit tube 3732-1 along a horizontal direction may be an acute angle, so as to form a space used to accommodate the gas filter unit 4810 between the gas intake tube 3731-1 and the gas outlet port of the main body of the ventilatory treatment apparatus 200.

In some embodiments, the gas intake tube 3731-1 may include a free end, and the gas filter unit 4810 may be coupled to the free end of the gas intake tube 3731-1. In some embodiments, one end of the gas filter unit 4810 (or the gas intake tube 3731-1) may include an inner cone connector, and one end of the gas intake tube 3731-1 (or the gas filter unit 4810) may include an outer cone connector. The inner cone connector and the outer cone connector may be fitted with each other to achieve a sealed connection. For example, an end of the gas intake tube 3731-1 facing the gas filter unit 4810 may include an inner cone surface, and an end of the gas filter unit 4810 may include an outer cone surface that is matched with the inner cone surface, so that the outer cone surface may be inserted into the inner cone surface of the gas intake tube 3731-1 to achieve a sealed connection, thereby ensuring a connection reliability and an air tightness. In some embodiments, a diameter of the gas intake tube 3731-1 may be 22 mm.

In some embodiments, as shown in FIG. 48B, one end of the gas exit tube 3732-1 away from the tank 3720 may include a first positioning hook 3732a. In some embodiments, the first positioning hook 3732a may be matched to a limiting slot in a positioning slot (e.g., the positioning slot 542 in FIG. 54A) that is disposed on the ventilatory treatment apparatus 200, so as to prevent the first connecting adapter 4800 from separating from the ventilatory treatment apparatus 200.

In some embodiments, the first positioning hook 3732a may be bent upward to hook the limiting slot. In some embodiments, the first positioning hook 3732a may hook the limiting slot through an elastic member (e.g., a spring). In some embodiments, a hooked length between the positioning hook 3732a and the limiting slot may be in a range of 0.5 mm-5 mm, so as to facilitate a disassembling of the tank 3720 from the main body of the ventilatory treatment apparatus 200. For example, a hooked length between the positioning hook 3732a and the limiting slot may be 1 mm.

FIG. 49 illustrates another exemplary connecting adapter according to some embodiments of the present disclosure.

In some embodiments, as shown in FIG. 49, a gas intake tube 3731-2 and a gas exit tube 3732-2 may be configured as an integral piece to form a second connecting adapter 4900. In some embodiments, the second connecting adapter 4900 may be separated from the tank 3720. In some embodiments, the second connecting adapter 4900 and the tank 3720 may be configured as an integral piece. In some embodiments, the second connecting adapter 4900 may be coupled to the tank 3720 in other feasible manners, which is not limited herein.

In some embodiments, the gas intake tube 3731-2 may be connected to the gas outlet port of the main body of the ventilatory treatment apparatus 200. In some embodiments, the tank 3720 may include a gas inlet port and a gas outlet port that are matched to the gas intake tube 3731-2 and the gas exit tube 3732-2 of the second connecting adapter 4900. For example, the gas inlet port and the gas outlet port may be disposed on the top of the tank 3720. In some embodiments, a distance between the gas intake tube 3731-2 of the second connecting adapter 4900 and the gas outlet port of the main body of the ventilatory treatment apparatus 200 may be less than a distance between the gas intake tube 3731-1 of the first connecting adapter 4800 and the gas outlet port of the main body of the ventilatory treatment apparatus 200. In some embodiments, a length of the gas intake tube 3731-2 of the second connecting adapter 4900 may be greater than a length of the gas intake tube 3731-1 of the first connecting adapter 4800.

In some embodiments, the tank 3720 may include a first gas inlet port matched to the gas intake tube 3731-1 of the first connecting adapter 4800 and/or a second gas inlet port matched to the gas intake tube 3731-2 of the second connecting adapter 4900. The first gas inlet port may be blocked when the second connecting adapter 4900 is coupled to the tank 3720, or the second gas inlet port may be blocked when the first connecting adapter 4800 is coupled to the tank 3720, so as to increase a distance between the first gas inlet port of the tank 3720 and the gas outlet port of the main body of the ventilatory treatment apparatus 200.

In some embodiments, the gas intake tube 3731-2 and the gas exit tube 3732-2 of the second connecting adapter 4900 may include an extending section along a horizontal direction, respectively. Extending directions of the extending sections may be perpendicular to a connection line of the gas inlet port and the gas outlet port disposed on the tank 3720.

In some embodiments, as shown in FIG. 49, one end of the gas exit tube 3732-2 of the second connecting adapter 4900 away from the tank 3720 may include a second positioning hook 3732b. More details about the second positioning hook 3732b may be found elsewhere in the present disclosure (e.g., FIG. 48B and related descriptions thereof).

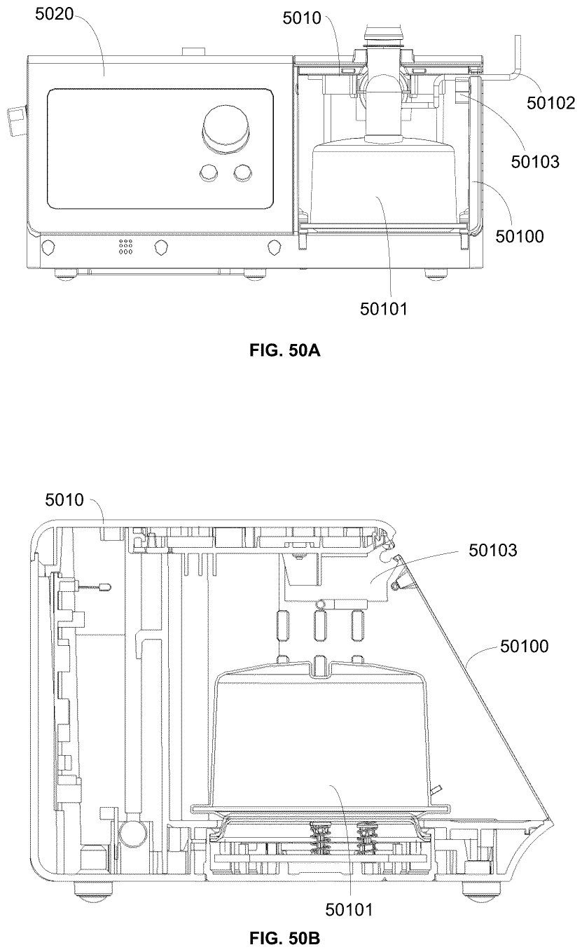

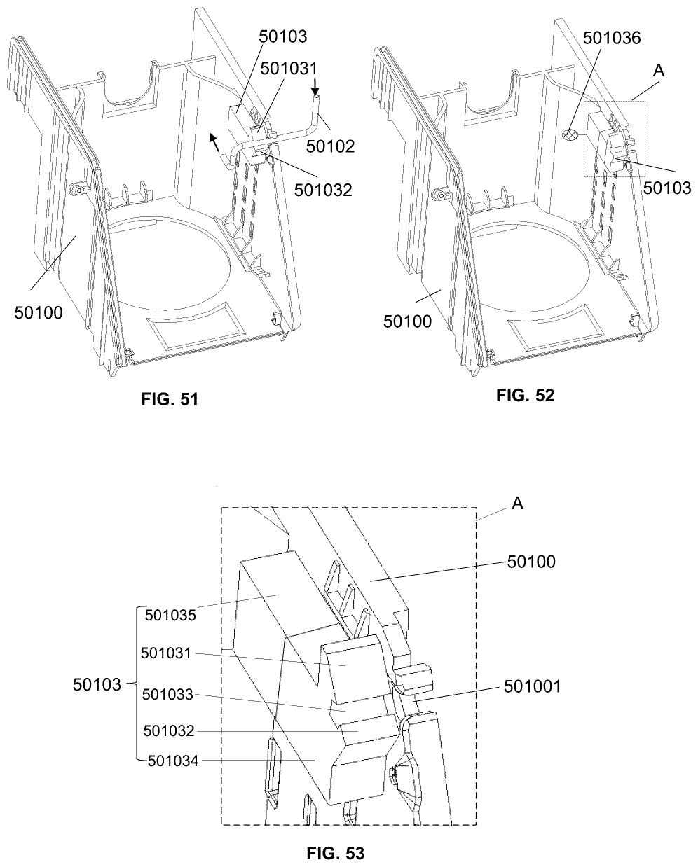

FIGS. 50A-50B illustrate an exemplary ventilatory treatment apparatus according to some embodiments of the present disclosure. FIG. 50A shows a front view of the ventilatory treatment apparatus. FIG. 50B shows a left view of the ventilatory treatment apparatus. FIG. 51 illustrates an exemplary liquid flow detection device and an exemplary liquid filling tube of the ventilatory treatment apparatus according to some embodiments of the present disclosure. FIG. 52 illustrates an exemplary liquid flow detection device with an alarming device according to some embodiments of the present disclosure. FIG. 53 illustrates an enlarged view of the liquid flow detection device shown in FIG. 52 according to some embodiments of the present disclosure.

In some embodiments, the ventilatory treatment apparatus (e.g., the ventilatory treatment apparatus 200) may include a main body 5020 and a humidification assembly 5010. The humidification assembly 5010 may be connected to the main body 5020. The main body 5020 may include a shell 50100. The humidification assembly 5010 may include a tank 50101. The shell 50100 may include an accommodation chamber. In some embodiments, the tank 50101 may be disposed in the accommodation chamber.

In some embodiments, the ventilatory treatment apparatus (e.g., the ventilatory treatment apparatus 200) may further include a liquid flow detection device 50103 and/or a liquid filling tube 50102. The liquid filling tube 50102 may be coupled to the tank 50101. The liquid filling tube 50102 may be configured to introduce the liquid to the tank 50101 to ensure that a certain amount of liquid may be maintained in the tank 50101. In some embodiments, the liquid filling tube 50102 may include a first end and a second end deviated from the first end. The first end of the liquid filling tube 50102 may be coupled to the tank 50101, and the second end of the liquid filling tube 50102 may be coupled to an external liquid source through a mounting hole (e.g., a mounting hole 501001 shown in FIG. 53) that penetrates a housing of the ventilatory treatment apparatus 200. The external liquid source may include a liquid bag, a liquid tank, a liquid storage apparatus, etc. The liquid filling tube 50102 may be used to introduce the liquid in the external liquid source to the tank 50101. In some embodiments, as shown in FIG. 50A and FIG. 51, the first end of the liquid filling tube 50102 may be horizontally arranged, and a segment of the second end of the liquid filling tube 50102 may be bent upward along a vertical direction. That is, the second end of the liquid filling tube 50102 may include a horizontal segment and/or a vertical segment.

In some embodiments, the liquid filling tube 50102 may be a rubber tube. For example, the liquid filling tube 50102 may be a transparent rubber tube. The transparent rubber tube may facilitate the use of a non-contact detection device for a real-time detection of the liquid flow in the liquid filling tube 50102, thereby avoiding a liquid pollution caused by the liquid flow detection device 50103. The transparent rubber tube may also facilitate a direct observation of the liquid flow in the liquid filling tube 50102, thereby ensuring an accuracy of the liquid flow monitoring. In some embodiments, the liquid filling tube 50102 may be made of a material including silicone material, transparent polypropylene material, transparent polyethylene material, other transparent rubber and plastic material, etc.

In some embodiments, a type of the liquid filling tube 50102 may be determined and/or selected based on the structure of the humidification assembly 5010 and/or the external liquid source, which is not limited herein.

In some embodiments, the liquid flow detection device 50103 may be disposed inside the shell 50100 of the ventilatory treatment apparatus 200 and opposite to the liquid filling tube 50102. In some embodiments, the liquid flow detection device 50103 may be disposed adjacent to the first end of the liquid filling tube 50102 coupled to the tank 50101. The liquid flow detection device 50103 may be configured to detect a change of the liquid flow in the liquid filling tube 50102. If the liquid flow detection device 50103 detects no liquid flow in the liquid filling tube 50102, which indicates the external liquid source may not include enough liquid, the external liquid source may need to be changed (or extra liquid may need to be added to the external liquid source) to prevent the tank 50101 from heating up without liquid. In some embodiments, an arrangement of the liquid flow detection device 50103 and the liquid filling tube 50102 may be determined based on a type of the liquid flow detection device 50103, which is not limited herein.

In some embodiments, the liquid flow detection device 50103 may include a liquid flow sensor, a liquid meter, a photoelectric sensor, or the like, or any combination thereof. In some embodiments, the liquid flow detection device 50103 may include a non-contact liquid meter detection device. For example, the liquid flow detection device 50103 may detect the liquid flow in the liquid filling tube 50102 based on an ultrasound and/or infrared light by using an ultrasonic flowmeter and/or infrared light sensor. In some embodiments, the liquid flow detection device 50103 may also include other types of liquid flow detection devices, which is not limited herein.

In some embodiments, the liquid flow detection device 50103 may include a photoelectric sensor. In some embodiments, the photoelectric sensor may be disposed on an inner surface of the shell 50100. In some embodiments, as shown in FIG. 51, the photoelectric sensor may include an optical transmitter 501031 and an optical receiver 501032. The optical transmitter 501031 may transmit an infrared light, an ultraviolet light, a visible light, or a light in other wavelength bands. The optical receiver 501032 may receive the light transmitted by the optical transmitter 501031. In some embodiments, the optical transmitter 501031 and the optical receiver 501032 may be disposed at two sides of the liquid filling tube 50102. For example, the liquid filling tube 50102 may be disposed between the optical transmitter 501031 and the optical receiver 501032 along a radial direction of the liquid filling tube 50102. A light transmitted by the optical transmitter 501031 may penetrate through the liquid filling tube 50102 and may be received by the optical receiver 501032. The optical receiver 501032 may determine the change of the liquid flow in the liquid filling tube 50102 based on a received optical signal.

The photoelectric sensor may detect a change of a light signal to determine the change of the liquid flow in the liquid filling tube 50102. When the photoelectric sensor detects that no liquid flows in the liquid filling tube 50102, the external liquid source may need to be replaced in time, thereby improving the safety of the apparatus. Merely by way of example, arrows shown in FIG. 51 may indicate a direction of the liquid flow in the liquid filling tube 50102. When the liquid flows in the liquid filling tube 50102, the light transmitted by the optical transmitter 501031 may penetrate through the liquid filling tube 50102, and part of the light may be scattered or absorbed by the liquid flow. The light received by the optical receiver 501032 may be reduced. The greater amount of the liquid flow in the liquid filling tube 50102, the more obvious the reduction of the light received by the optical receiver 501032. When there is no liquid flowing the liquid filling tube 50102, the light received by the light receiver 501032 may not be reduced relative to that transmitted by the optical transmitter 501031. That is, whether there is liquid flowing in the liquid filling tube 50102 may be determined based on a change of an optical signal received by the optical receiver 501032 relative to an optical signal transmitted by the optical transmitter 501031, thereby detecting a change of the liquid flow in the humidification assembly 3700.

It should be noted that the change of the liquid flow in the liquid filling tube 50102 that is detected by the photoelectric sensor may achieve a non-contact detection of the liquid flow in the liquid filling tube 50102, thereby achieving a real-time detection of the liquid flow and avoiding liquid pollution. In some embodiments, a type of the photoelectric sensor may be determined based on actual requirements, which is not limited herein.

In some embodiments, as shown in FIG. 53, the photoelectric sensor may include a slot 501033. The optical transmitter 501031 and the optical receiver 501032 may be disposed at two sides of the slot 501033. The liquid filling tube 50102 may penetrate through the slot 501033 and stuck in the slot 501033, thereby achieving a relative fixation of the liquid filling tube 50102 and the photoelectric sensor, and facilitating the usage and installation of the liquid filling tube 50102. The liquid filling tube 50102 fixed by the slot 501033 may also avoid a relative movement of the liquid filling tube 50102, so as to improve an accuracy of the detection result. The light transmitted by the optical transmitter 501031 may penetrate through the liquid filling tube 50102 that is stuck in the slot 501033, and at least a part of the light transmitted by the optical transmitter 501031 may be received by the optical receiver 501032.

In some embodiments, a structure and a size of the slot 501033 may be matched to a structure and a size of the liquid filling tube 50102, so as to ensure a relative fixation of the liquid filling tube 50102 and the photoelectric sensor. In some embodiments, a structure and a size of the slot 501033 may be determined based on a structure and a size of the liquid filling tube 50102, which is not limited herein.

In some embodiments, as shown in FIG. 53, the slot 501033 may be aligned with the mounting hole 501001. For example, the photoelectric sensor may be disposed on a sidewall of the shell 50100, and the slot 501033 of the photoelectric sensor may be aligned with the mounting hole 501001 disposed on the sidewall of the shell 50100, thereby facilitating the installation of the liquid filling tube 50102 and improving a sensitivity of detecting the change of the liquid flow.

Merely by way of example, the liquid in a liquid bag disposed outside of the shell 50100 may be introduced into the tank 50101 through the liquid filling tube 50102. That is, the liquid in the liquid filling tube 50102 may flow from the liquid bag to the tank 50101. The part of the liquid filling tube 50102 stuck in the mounting hole 501001 may be closer to the liquid bag. When there is no liquid in the liquid bag, the second end (e.g., the horizontal segment of the second end) of the liquid filling tube 50102 close to the liquid bag may not be filled with the liquid. That is, the change of the liquid flow in the liquid filling tube 50102 may be early detected by arranging the photoelectric sensor close to the mounting hole 501001, thereby replacing the liquid bag in time and improving the sensitivity of detecting the change of the liquid flow.

In some embodiments, the liquid filling tube 50102 may penetrate through the housing of the shell 50100 and the photoelectric sensor by aligning the slot 501033 and the mounting hole 501001, thereby simplifying an installation process and improving the operation convenience.

In some embodiments, a location of the mounting hole 501001 may be determined based on actual requirements, and a location of the photoelectric sensor may be determined based on the location of the mounting hole 501001, which is not limited herein.

In some embodiments, the photoelectric sensor may be disposed close to the first end of the liquid filling tube 50102, so that the photoelectric sensor may be close to the tank 50101, thereby determining the amount of the liquid in the tank 50101 accurately based on the change of the liquid flow detected by the photoelectric sensor.

It should be noted that a preset amount liquid may be required to be kept in the tank 50101. When the liquid in an external liquid source (e.g., the liquid bag) just finished flowing from the external liquid source to the tank 50101, a certain amount of liquid may be kept in the liquid filling tube 50102. A total amount of the liquid in the tank 50101 may be a summation of the preset amount liquid in the tank 50101 and the certain amount of liquid in the liquid filling tube 50102. The photoelectric sensor may determine the total amount of the liquid in the tank 50101 to provide basis for subsequent operations.

In some embodiments, as shown in FIG. 53, the photoelectric sensor may also include a converter 501034. The converter 501034 may be connected with the optical receiver 501032. The converter 501034 may be configured to convert an optical signal received by the optical receiver 501032 to an electrical signal. A controller (a controller 501035 shown in FIG. 53) may determine the change of the liquid flow in the liquid filling tube 50102 based on a change of the electrical signal, thereby facilitating a real-time detection of the change of the liquid flow in the liquid filling tube 50102 and achieving an obvious detection result. The external liquid source may be replaced in time to prevent the tank 50101 from heating up without liquid, so as to improve the safety of the ventilatory treatment apparatus.

In some embodiments, the optical transmitter 501031, the optical receiver 501032, and the converter 501034 may be configured as an integral piece, so as to facilitate a detachment of the photoelectric sensor and save a mounting space. In some embodiments, the optical transmitter 501031, the optical receiver 501032, and the converter 501034 may be independent from each other, so as to be disposed based on actual requirements, which is not limited herein.

In some embodiments, as shown in FIG. 53, the liquid flow detection device 50103 may further include the controller 501035. The controller 501035 may be connected with the converter 501034. The controller 501035 may be configured to determine the change of the liquid flow in the liquid filling tube 50102 based on the electrical signal, so as to obtain an intuitive detection result.

Merely by way of example, a voltage may be converted by the converter 501034. When the liquid flows in the liquid filling tube 50102, the light transmitted by the optical transmitter 501031 may penetrate through the liquid filling tube 50102, and part of the light may be scattered or absorbed by the liquid, so that the light received by the optical receiver 501032 may be reduced, and the voltage converted by the converter 501034 that is connected with the optical receiver 501032 may be reduced. When there is no liquid flowing in the liquid filling tube 50102, the light received by the optical receiver 501032 may not be reduced, and the voltage converted by the converter 501034 may increase. That is, the controller 501034 may determine the change of the liquid flow in the liquid filling tube 50102 based on a change of the voltage converted by the converter 501034.

In some embodiments, when the electrical signal converted by the converter 501034 is another type of electrical signal, the controller 501034 may determine the change of the liquid flow in the liquid filling tube 50102 based on a change of the electrical signal, which is not limited herein.

In some embodiments, as shown in FIG. 52, the liquid flow detection device 50103 may further include an alarming device 501036. The alarming device 501036 may be connected to the controller 501035. The alarming device 501036 may be controlled by the controller 501035 to generate an alarming signal when the change of the liquid flow is greater than a preset range. The external liquid source may be replaced in time to prevent the tank 50101 from heating up without liquid based on the alarming signal, so as to improve the safety of the ventilatory treatment apparatus. In some embodiments, the preset range of the change of the liquid flow may be determined based on the change of the electrical signal.

Merely by way of example, when the electrical signal is a voltage and the liquid flows in the liquid filling tube 50102, the voltage converted by the converter 501034 may fluctuate within a certain range. The certain range may be determined in advance and input into the controller 501035. The controller 501035 may acquire the voltage converted by the converter 501034 in real-time. In response to determining that the voltage exceeds the preset range, the controller 501035 may control the alarming device 501036 to generate the alarming signal. In some embodiments, types of the electrical signals converted by different types of converters may also be different, which is not limited herein.

In some embodiments, the preset range of the electrical signal may be determined in advance based on the change of the liquid flow in the liquid filling tube 50102. In some embodiments, the preset range of the electrical signal may be determined based on actual requirements, which is not limited herein.

In some embodiments, the alarming device 501036 may be disposed inside the shell 50100. In some embodiments, the alarming device 501036 may be disposed on a sidewall of the shell 50100.

In some embodiments, the liquid flow detection device 50103 may include a contact detection device and a non-contact detection device. The non-contact detection device may include an ultrasonic flowmeter, an infrared light sensor, etc., so as to avoid the liquid pollution caused by a direct contact of the liquid flow detection device 50103 and the liquid filling tube 50102.

In some embodiments, the photoelectric sensor may be an infrared photoelectric sensor. The infrared photoelectric sensor may detect the change of the liquid flow in the liquid filling tube 50102 based on an infrared light penetrating the liquid filling tube 50102. The infrared photoelectric sensor may convert the change of light signal of the infrared light into the change of the liquid flow based on a photoelectric effect.

The infrared light with a better anti-interference ability may improve an accuracy of a detection result determined by the photoelectric sensor. The infrared photoelectric sensor may detect the change of the liquid flow in the liquid filling tube through a non-contact detection manner, thereby avoiding liquid contamination during the detection and improving the security of the apparatus. In some embodiments, the photoelectric sensor may be other types of sensors, which is not limited herein.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIGS. 55A-55C illustrate an exemplary heater assembly according to some embodiments of the present disclosure. FIG. 55A shows an overall view of the heater assembly 5500. FIG. 55B shows a top view of the heater assembly 5500. FIG. 55C shows a side view of the heater assembly 5500.

The heater assembly 5500 may be configured to heat a liquid (e.g., water in the humidification assembly 206). The liquid may be accommodated in the liquid chamber of the humidification assembly 206. In some embodiments, the heater assembly 5500 may be applied in a ventilatory treatment apparatus (e.g., the ventilatory treatment apparatus 200 shown in FIGS. 2A-2F). In some embodiments, the heater assembly 5500 may be detachably coupled to the ventilatory treatment apparatus. In some embodiments, the heater assembly 5500 and the ventilatory treatment apparatus may be configured as an integral piece. In some embodiments, the heater assembly 5500 may be coupled to the ventilatory treatment apparatus in other feasible manners, which is not limited herein.

In some embodiments, as shown in FIGS. 55A-55C, the heater assembly 5500 may include a heat transfer plate 5510. The heat transfer plate 5510 may be configured to heat the liquid and generate vapor to humidify a gas (e.g., the resparitery gas) above the liquid. The humidified gas may be an appropriate breathing gas provided to the subject 180.

In some embodiments, the heat transfer plate 5510 may be fitted with the tank bottom 3722. A shape (and/or a size) of the heat transfer plate 5510 may be matched to a shape (and/or a size) of the tank bottom 3722. In some embodiments, the heat transfer plate 5510 may be in a shape of circle. In some embodiments, an edge of the heat transfer plate 5510 along a circumferential direction may be sloped to accommodate one or more components (e.g., the heater 5610, the overheat protection mechanism 5620, and/or the fastening piece 5630) disposed underneath the heat transfer plate 5510. In some embodiments, the heat transfer plate 5510 may be in other shapes, such as in a shape of square, rectangle, etc., which is not limited herein.

In some embodiments, the heat transfer plate 5510 may be made of a thermally conductive material. The thermally conductive material may include copper, aluminum, aluminum nitride, silicon carbide, tungsten, graphite, zinc, etc., which is not limited herein. For example, the heat transfer plate 5510 may include a metal plate.

In some embodiments, the heater assembly 5500 may be connected to a power supply via one or more power lines 5520. The power supply may provide power to the heater assembly 5500. The power line(s) 5520 may be configured to transmit power (provided by the power supply) to the heater assembly 5500. In some embodiments, the power supply may provide an alternating current (AC) voltage, and a value of the AC voltage may be 110 V or 220 V. In some embodiments, the power supply may include an external power supply, an internal power supply of the apparatus, etc. Exemplary power supply may include the power supply assembly 60100 (specifically, the non-isolated power supply component 60130 of the power supply assembly 60100).

In some embodiments, the heater assembly 5500 may be connected to a control circuit via one or more control line(s) 5530. The control line(s) 5530 may be configured to transmit one or more control signals from the control circuit to the heater assembly 5500, so as to control a working status of the heater assembly 5500. For example, the control line(s) 5530 may be used to transmit a drive signal generated by the control circuit. In some embodiments, the drive signal may be generated based on a voltage (or the drive signal may include the voltage signal) used to keep a power of the heater assembly 5500 constant. In some embodiments, the control line(s) 5530 may be connected to a controller of the control circuit to transmit control signal(s) (used to control a temperature of the heater assembly 5500) determined by the controller.

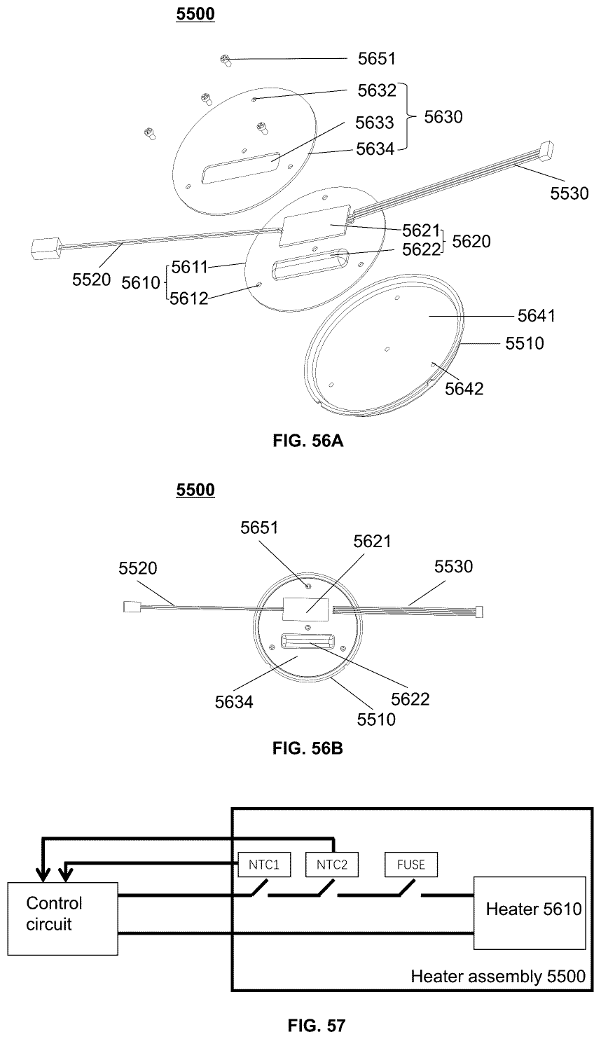

FIGS. 56A-56B illustrate rear views of the heater assembly 5500 shown in FIGS. 55A-55C according to some embodiments of the present disclosure. FIG. 56A shows an exploded view of the heater assembly 5500. FIG. 56B shows a bottom view of the heater assembly 5500.

In some embodiments, as shown in FIG. 56A, the heater assembly 5500 may further include a heater 5610 and an overheat protection mechanism 5620. The heater 5610 may be configured to heat the heat transfer plate 5510. The overheat protection mechanism 5620 may be configured to protect the heater 5610 from overheating.

In some embodiments, the heater 5610 may include a heating pad 5611 and one or more through-holes 5612. The heating pad 5611 may be configured to heat the transfer plate 5510. In some embodiments, the heating pad 5611 may be further configured to carry the overheat protection mechanism 5620. The through-hole(s) 5612 may be disposed on the heating pad 5611. In some embodiments, a count of the through-hole(s) 5612 disposed on the heating pad 5611 may be four, or any other number. In some embodiments, the through-hole(s) 5612 may be disposed at intervals on an edge of the heating pad 5611 along a circumferential direction. In some embodiments, at least one of the through-hole(s) 5612 may coincide with a center of the heating pad 5611, and remaining through-hole(s) 5612 of the through-hole(s) 5612 may be disposed at intervals on the edge of the heating pad 5611 along the circumferential direction. For example, a through-hole 5612 may coincide with the center of the heating pad 5611, and three through-holes 5612 may be disposed on the edge of the heating pad 5611 so as to form a triangle, and distances from the three through-holes 5612 disposed on the edge of the heating pad 5611 to the through-hole 5612 located at the center of the heating pad 5611 may be (substantially) equal. As used herein, substantially, when used to qualify a feature (e.g., equivalent to, the same as, etc.), indicates that the deviation from the feature is below a threshold, e.g., 30%, 25%, 20%, 15%, 10%, 5%, etc.

In some embodiments, the heating pad 5611 may be in a shape that is fitted with a shape of an inner surface 5641 of the heat transfer plate 5510. For example, the heating pad 5611 may be in a shape of circle. A diameter of the heating pad 5611 may be (substantially) equal to a diameter of the inner surface 5641 of the heat transfer plate 5510. In some embodiments, the heating pad 5611 may be in a shape of polygon, such as square, rectangle, etc., which is not limited herein. In some embodiments, the heating pad 5611 may be made of an organic high-temperature resistant material. For example, the heating pad 5611 may include a silicone heating pad. In some embodiments, the heating pad 5611 may be made of other high-temperature resistant materials, which is not limited herein.

In some embodiments, as shown in FIG. 56A, the heater assembly 5500 may further include a fastening piece 5630. The fastening piece 5630 may be configured to fasten the heater 5610 and the overheat protection mechanism 5620 onto the heat transfer plate 5510. In some embodiments, the fastening piece 5630 may include one or more through-holes 5632, one or more openings 5633, and/or a rigid plate 5634. In some embodiments, the through-hole(s) 5632 may be disposed on the rigid plate 5634. The size, shape, and/or distribution of the through-holes 5632 may be the same as or similar to the through-holes 5612. More details about the through-holes 5632 may refer to FIG. 56A and related descriptions of the through-holes 5612 thereof. The opening(s) 5633 may be disposed on the rigid plate 5634. The opening(s) 5633 may be configured to provide space for the overheat protection mechanism 5620.

In some embodiments, the rigid plate 5634 may be configured to press over the overheat protection mechanism 5620. In some embodiments, the rigid plate 5634 may be in a shape that is fitted with the shape of the inner surface 5641 of the heat transfer plate 5510. For example, the rigid plate 5634 may be in a shape of circle. A diameter of the rigid plate 5634 may be (substantially) equal to the diameter of the inner surface 5641 of the heat transfer plate 5510. In some embodiments, the rigid plate 5634 may be in a shape of polygon, such as square, rectangle, etc., which is not limited herein. In some embodiments, the rigid plate 5634 may be made of a metal material or a non-metal material. For example, the rigid plate 5634 may include a steel plate with a high strength. As another example, the rigid plate 5634 may be made of a non-metal material such as nylon, polycarbonate (PC), etc. In some embodiments, the rigid plate 5634 may be made of other rigid materials, which is not limited herein.

In some embodiments, as shown in FIG. 56A, the heat transfer plate 5510 may further include one or more holes 5642. In some embodiments, the holes 5642 may be non-through holes. In some embodiments, the holes 5642 may be through-holes. The holes 5642 may be disposed on the inner surface 5641 of the heat transfer plate 5510. The size, shape, and/or distribution of the holes 5642 may be the same as or similar to the through-holes 5612. More details about the through-holes 5642 may refer to FIG. 56A and related descriptions of the through-holes 5612 thereof. In some embodiments, the hole(s) 5642 may have internal thread. The fastening piece 5630, the heater 5610 and the overheat protection mechanism 5620, may be fixed onto the heat transfer plate 5510 using one or more screw nails 5651. The screw nail(s) 5651 may pass through the through hole(s) 5632 of the fastening piece 5630, the through hole(s) 5612 of the heater 5610, and fixedly coupled to the hole(s) 5642 of the heat transfer plate 5510. A count of the screw nail(s) 5651 may be equal to a count of the through-hole(s) 5632 (or the through hole(s) 5612, or the hole(s) 5642). For example, the count of the screw nails 5651 and the through-holes 5632 may be four, respectively.

In some embodiments, as shown in FIG. 56B, the fastening piece 5630 may be fixed onto the heat transfer plate 5510. In some embodiments, the fastening piece 5630 may be fixed onto the heat transfer plate 5510 through a threaded connection as illustrated above. For example, the count of the through-holes 5632 disposed on the rigid plate 5634 may be equal to the count of the holes 5642 disposed on the inner surface 5641 of the heat transfer plate 5510, and positions of the through-holes 5632 disposed on the rigid plate 5634 may (substantially) correspond to positions of the holes 5642 disposed on the inner surface 5641 of the heat transfer plate 5510, respectively. The through-holes 5632 disposed on the rigid plate 5634 may be aligned with the holes 5642 disposed on the inner surface 5641 of the heat transfer plate 5510, and the screw nails 5651 may penetrate through the through-holes 5632 and into the holes 5642 and tightened so that the fastening piece 5630 is fixed onto the heat transfer plate 5510. In some embodiments, the fastening piece 5630 may be fixed onto the heat transfer plate 5510 through other feasible connection manners, such as thermocompression bonding, etc., which is not limited herein.

In some embodiments, the heater 5610 and the overheat protection mechanism 5620 may be sandwiched between the fastening piece 5630 and the heat transfer plate 5510. For example, the heater 5610 and the overheat protection mechanism 5620 may be coupled to the heat transfer plate 5510 through thermocompression bonding, and the fastening piece 5630 may cover the heater 5610 and the overheat protection mechanism 5620 and may be fixed onto the heat transfer plate 5510. As another example, the heater 5610 and the overheat protection mechanism 5620 may be sandwiched between the fastening piece 5630 and the heat transfer plate 5510 through a threaded connection. As shown in FIG. 56A, the count of through-holes 5612, through-holes 5632, and holes 5642 may be equal, and the positions of the through-holes 5612, through-holes 5632, and holes 5642 may (substantially) correspond to each other, respectively. The screw nails 5651 may penetrate through through-holes 5612, through-holes 5632, and into the holes 5642 and tightened so that the fastening piece 5630 may cover the heater 5610 and the overheat protection mechanism 5620, and the overheat protection mechanism 5620 may be pressed onto the heating pad 5611, and the heating pad 5611 may be fixed onto the heat transfer plate 5510.

Impurities entering between the overheat protection mechanism 5620 and the heat transfer plate 5510 may cause hollow drum(s) between the overheat protection mechanism 5620 and the heat transfer plate 5510. That is, a gap may be formed between the overheat protection mechanism 5620 and the heat transfer plate 5510 due to the impurities, which causes a deviation between a detected temperature of the heat transfer plate 5510 determined by the overheat protection mechanism 5620 and an actual temperature of the heat transfer plate 5510, thereby having a negative effect on an overheat protection performance of the overheat protection mechanism 5620. The heater 5610 and the overheat protection mechanism 5620 may be sandwiched between the fastening piece 5630 and the heat transfer plate 5510 through the thermocompression bonding, the threaded connection, etc., so that the overheat protection mechanism 5620 may be tightly attached to the heat transfer plate 5510 through the fastening piece 5630, thereby preventing impurities from entering between the overheat protection mechanism 5620 and the heat transfer plate 5510, ensuring an accurate detected temperature determined by the overheat protection mechanism 5620, and improving the overheat protection performance of the overheat protection mechanism 5620.

In some embodiments, as shown in FIG. 56A, the overheat protection mechanism 5620 may include a temperature sensor unit 5621 and a fuse 5622. The temperature sensor unit 5621 may be configured to detect a temperature of the heat transfer plate 5510. The fuse 5622 may be configured to provide overcurrent protection of the heater 5610 through fusing.

In some embodiments, the temperature sensor unit 5621 and the fuse 5622 may be configured as a multi-level overheat protection mechanism, and the overheat protection mechanism 5620 may provide overheat protection of the heater 5610 level by level. In some embodiments, the multi-level overheat protection mechanism may include protection through software control, hardware control, etc.

In some embodiments, the temperature sensor unit 5621 may include a first temperature sensor. The first temperature sensor may be configured to detect a temperature of the heat transfer plate 5510. In some embodiments, the overheat protection mechanism 5620 may provide a first level overheat protection of the heater 5610 based on the first temperature sensor. The first level overheat protection may include software control.

In some embodiments, when the first temperature sensor detects the temperature of the heat transfer plate 5510 exceeds a first threshold, the overheat protection mechanism 5620 may protect the heater 5610 from overheating through the software control. For example, the first temperature sensor may detect a temperature of the heat transfer plate 5510 and generate a first signal corresponding to the detected temperature. The temperature sensor unit 5621 may transmit the first signal to a heating control logic of the control circuit through the control line(s) 5530. The heating control logic of the control circuit may determine the temperature of the heat transfer plate 5510 based on the first signal and determine whether the temperature of the heat transfer plate 5510 exceeds the first threshold. In response to a determination that the temperature of the heat transfer plate 5510 exceeds the first threshold, the software control may be performed by transmitting a control signal generated by the heating control logic of the control circuit to the heater 5610 to stop the heating of the heater 5610. In some embodiments, the first signal may include a voltage signal.

In some embodiments, the temperature sensor unit 5621 may further include a second temperature sensor. The second temperature sensor may be configured to detect a temperature of the heat transfer plate 5510. In some embodiments, the overheat protection mechanism 5620 may provide a second level overheat protection of the heater 5610 based on the second temperature sensor. The second level overheat protection may include hardware control.

In some embodiments, when the second temperature sensor detects the temperature of the heat transfer plate 5510 exceeds a second threshold, the overheat protection mechanism 5620 may protect the heater 5610 from overheating through the hardware control. For example, the second temperature sensor may detect a temperature of the heat transfer plate 5510 and generate a second signal corresponding to the detected temperature. The temperature sensor unit 5621 may transmit the second signal to a comparator of the control circuit through the control line(s) 5530. The control circuit may determine a reference signal corresponding to the second threshold and determine whether the temperature of the heat transfer plate 5510 exceeds the second threshold by comparing the second signal with the reference signal through the comparator. In response to a determination that the temperature of the heat transfer plate 5510 exceeds the second threshold, the hardware control may be performed by using the comparator to cut off the heating control logic of the control circuit.

In some embodiments, the second signal may include a voltage signal, and the comparator may include a voltage comparator. For example, the hardware control may be performed by using a voltage comparator to cut off the heating control logic of the control circuit. When a second voltage signal is less than or equal to a reference voltage signal, the voltage comparator may determine or output a high-level signal. When the second voltage signal is greater than the reference voltage signal, the voltage comparator may determine or output a low-level signal. The voltage comparator may cut off the heating control logic of the control circuit based on the low-level signal, and the heater 5610 may stop heating. The heater 5610 may be or keep in a normal working status based on the high-level signal.

In some embodiments, the first temperature sensor and/or the second temperature sensor may include a negative temperature coefficient (NTC) thermistor. The NTC thermistor may be configured to detect a temperature of the heat transfer plate 5510. In some embodiments, a resistance value of the NTC thermistor may be changed with a temperature change of the heat transfer plate 5510, so that a signal determined or output by the temperature sensor unit 5621 may be changed with the change of the resistance value of the NTC thermistor, and the overheat protection mechanism 5620 may provide the overheat protection to the heater 5610 based on the change of the signal determined by the temperature sensor unit 5621.

In some embodiments, the overheat protection mechanism 5620 may provide a third level overheat protection of the heater 5610 based on the fuse 5622. In some embodiments, when the temperature of the heat transfer plate 5510 exceeds a third threshold, the overheat protection mechanism 5620 may protect the heater 5610 from overheating by fusing the fuse 5622 to provide overcurrent protection of the heater 5610 through fusing.

In some embodiments, as shown in FIGS. 56A and 56B, the opening(s) 5633 of the rigid plate 5634 may be fitted with the temperature sensor unit 5621 and/or the fuse 5622. An exposed portion of the temperature sensor unit 5621 and/or the fuse 5622 may be covered with an insulation material, such as insulation tape, etc.

FIG. 57 illustrates a circuit connection of an overheat protection mechanism according to some embodiments of the present disclosure.

In some embodiments, as shown in FIG. 57, the heater 5610, the fuse 5622, the first temperature sensor (e.g., NTC1 shown in FIG. 57), the second temperature sensor (e.g., NTC2 shown in FIG. 57), and the control circuit may form a circuit loop. The first temperature sensor and the second temperature sensor may be connected to the control circuit to transmit the first signal and the second signal to the control circuit, respectively.

In some embodiments, when the first temperature sensor detects the temperature of the heat transfer plate 5510 exceeds the first threshold, the overheat protection mechanism 5620 may perform the first level overheat protection by cutting off a connection between the first temperature sensor and the control circuit to stop the heating of the heater 5610. When the first level overheat protection fails and the second temperature sensor detects the temperature of the heat transfer plate 5510 exceeds the second threshold, the overheat protection mechanism 5620 may perform the second level overheat protection by cutting off a connection between the second temperature sensor and the control circuit to stop the heating of the heater 5610. When the first level overheat protection and the second level overheat protection fail, and the temperature of the heat transfer plate 5510 exceeds the third threshold, the overheat protection mechanism 5620 may perform the third level overheat protection by cutting off a connection between the fuse 5622 and the control circuit to provide the overcurrent protection of the heater 5610 through fusing the fuse 5622.

In some embodiments, the control circuit may be configured to control a working status of the heater 5610. The control circuit may include a controller and a switch. The controller may be configured to generate a control signal to control a turn on or turn off of the switch, so as to control the working status of the heater 5610. In some embodiments, the control signal may include a turning on signal and a turning off signal, and the switch may include a metal-oxide-semiconductor field-effect transistor (MOSFET). For example, when the controller generates a turning on signal, the MOSFET may be turned on, and the heater 5610 may start heating; or when the controller generates a turning off signal, the MOSFET may be turned off, and the heater 5610 may stop heating.

In some embodiments, when the controller generates the turning on signal, the switch may be turned on, and the heater 5610 may start working for a preset period. In some embodiments, when the controller generates the turning off signal, the switch may be turned off, and the heater 5610 may stop working for a preset period.

In some embodiments, the controller may also receive a signal corresponding to a temperature of the heat transfer plate 5510 (i.e., also referred to a temperature signal) generated by the temperature sensor unit 5621. The temperature signal may be used to indicate a current temperature of the heat transfer plate 5510.

In some embodiments, the temperature sensor unit 5621 may transmit the temperature signal to the controller, and the controller may determine whether the current temperature of the heat transfer plate 5510 exceeds a threshold (e.g., the first threshold, the second threshold, and/or the third threshold) based on the temperature signal. In response to determining that the current temperature of the heat transfer plate 5510 does not exceed the threshold, the controller may generate the control signal to control the working status of the heater 5610 as mentioned above. In response to determining that the current temperature of the heat transfer plate 5510 exceeds the threshold, the controller may generate the turning off signal continually, so that the heater 5610 may stop working temporarily until the current temperature of the heat transfer plate 5510 being less than or equal to the threshold.

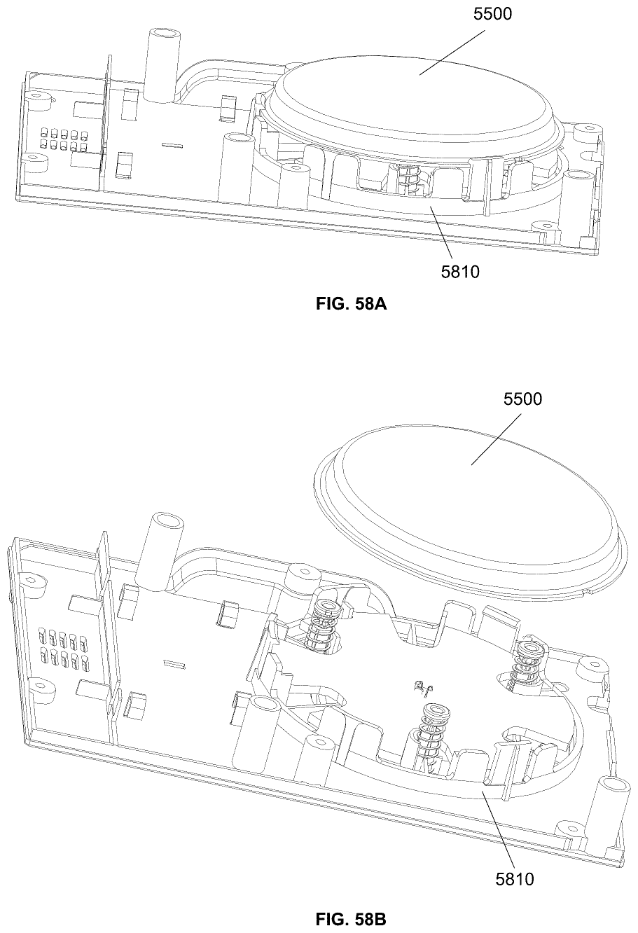

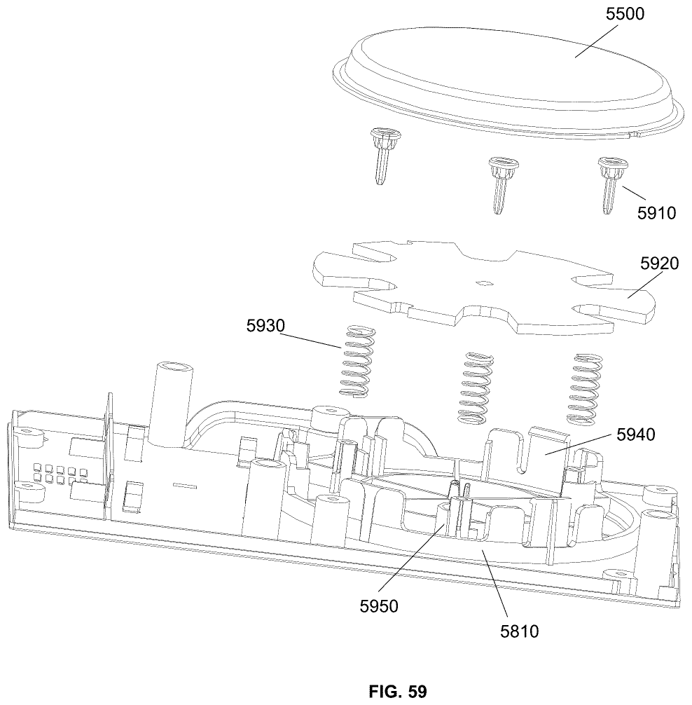

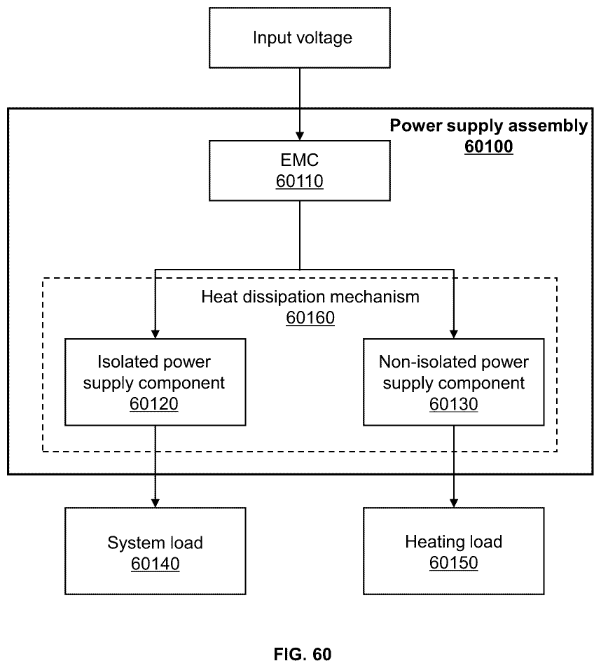

FIGS. 58A-58B illustrate an exemplary heater assembly mounted on a base of a ventilatory treatment apparatus according to some embodiments of the present disclosure. FIG. 58A shows an overall view of the heater assembly 5500 in FIGS. 55A-56B mounted on the base 5810 of a ventilatory treatment apparatus. FIG. 58B shows the heater assembly 5500 detached from the base 5810. FIG. 59 illustrates an exploded view of the heater assembly mounted on the base shown in FIGS. 58A-58B according to some embodiments of the present disclosure.

In some embodiments, the heater assembly 5500 may be mounted on a base 5810 of an ventilatory treatment apparatus (e.g., the ventilatory treatment apparatus 200 shown in FIG. 2). The base 5810 (also be referred to as a baseplate) may be configured to support and fix the heater assembly 5500. In some embodiments, as shown in FIG. 58A, the heater assembly 5500 may be mounted on the base 5810 to avoid a movement of the heater assembly 5500 relative to the ventilatory treatment apparatus. In some embodiments, as shown in FIG. 58B, the heater assembly 5500 may be detached from the base 5810.

In some embodiments, as shown in FIG. 59, the base 5810 may include one or more limit structures 5940. The limit structure(s) 5940 may be configured to limit a movement of the heater assembly 5500, so that the heater assembly 5500 may not be separated from the base 5810 arbitrarily. In some embodiments, the heater assembly 5500 may be pushed towards the base 5810 to be limited by the limit structure(s) 5940. In some embodiments, the base 5810 may further include one or more limit slots 5950 or limit posts configured to accommodate one or more elastic members 5930. The elastic member(s) 5930 may be configured to support and/or fix the heater assembly 5500. For example, the elastic member(s) 5930 may push the heater assembly 5500 towards the limit structure(s) 5940, and the limit structure(s) 5940 may restrict a movement of the heater assembly 5500. In some embodiments, the elastic member(s) 5930 may include one or more springs. In some embodiments, one or more heat insulation nails 5910 may be disposed in the elastic member(s) 5930. In some embodiments, a heat insulation part 5920 may be disposed between the heater assembly 5500 and the base 5810. The heat insulation nail(s) 5910 and the heat insulation part 5920 may be configured to reduce a heat conduction between the base 5810 and the heater assembly 5500.

In some embodiments, the heater assembly 5500 may be mounted on the base 5810 through the one or more elastic members 5930 and the one or more limit structures 5940. The one or more elastic members 5930 and the one or more limit structures 5940 may be disposed on the base 5810. In some embodiments, each of the one or more limit structures 5940 may include a buckle and a positioning surface. The positioning surface and the buckle may be opposite to each other. In some embodiments, the heater assembly 5500 may be capable of moving up and down upon being driven by a pressure or upon releasing the pressure (e.g., a pressure of the one or more elastic members 5930, a pressure imposed by an operator). For example, the heater assembly 5500 may move up and down between one or more buckles and one or more positioning surfaces based on the pressure. When the heater assembly 5500 is not pressed (e.g. the humidification assembly 206 is not placed on the heater assembly 5500), the heater assembly 5500 may move upward and abut against the one or more buckles, and the resistance of the buckle(s) and the pushing force of the elastic member(s) 5930 may be balanced, and thus, the heater assembly 5500 is stable. When the heater assembly 5500 is pressed (e.g., the humidification assembly 206 is placed on the heater assembly 5500), the heater assembly 5500 may move downward and abut against the one or more positioning surfaces, and the pressure of the humidification assembly 206 and the pushing force of the elastic member(s) 5930 may be balanced, and thus the heater assembly 5500 is stable.

In some embodiments, at least a portion of the heater assembly 5500 may be separated from the base 5810 of the apparatus by the heat insulation part 5920. The insulation part 5920 may be disposed between the heater assembly 5500 and the base 5810 to reduce the heat conduction between the heater assembly 5500 and the base 5810, so as to avoid a damage of the base 5810 caused by a high temperature.

In some embodiments, the insulation part 5920 may be made of an insulation material, such as an asbestos, a rock wool, an aerogel felt, a vacuum board, etc., which is not limited herein. The insulation part 5920 may include an insulation pad, an insulation foam, an insulation board, etc. In some embodiments, the insulation part 5920 may be in a shape matched to the base 5810. For example, the insulation part 5920 may be in a shape including a plurality of fan blades.

In some embodiments, the one or more heat insulation nails 5910 may be matched to the one or more elastic members 5930, respectively. The one or more insulation nails 5910 may be configured to reduce the heat conduction between the heater assembly 5500 and the base 5810 through the one or more elastic members 5930. For example, as shown in FIG. 59, each of the one or more springs may enclose a heat insulation nail 5910 to reduce the heat conducted from the heater assembly 5500 to the base 5810 through the one or more springs. In some embodiments, a count of the one or more elastic members 5930 may be three.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 60 illustrates an exemplary power supply assembly according to some embodiments of the present disclosure.

As illustrated in FIG. 60, the power supply assembly 60100 may include an isolated power supply component 60120, and a non-isolated power supply component 60130. In some embodiments, the power supply assembly 60100 may include an electromagnetic compatibility (EMC) circuit 60110. In some embodiments, the power supply assembly 60100 may include a heat dissipation mechanism 60160. In some embodiments, the isolated power supply component 60120 and the non-isolated power supply component 60130 may be integrated in a single circuit board and share at least a portion of an electromagnetic compatibility (EMC) circuit 60110. In some embodiments, the isolated power supply component 60120 and the non-isolated power supply component 60130 may share at least a portion of a heat dissipation mechanism 60160. In some embodiments, the isolated power supply component 60120 and the non-isolated power supply component 60130 may be integrated in different circuits, respectively.

The EMC circuit 60110 may refer to a circuit with an electromagnetic compatibility. The electromagnetic compatibility is an ability that an apparatus (e.g., the ventilatory treatment apparatus 200) or a system can satisfy certain requirements when the apparatus or the system is running in a certain electromagnetic environment, and the apparatus or the system may not produce intolerable electromagnetic interference (EMI) to any other devices in the certain electromagnetic environment. Thus, the EMC circuit 60110 may have two performances. A first performance may include that elements in the EMC circuit 60110 may not produce EMI exceeding a threshold. The threshold may be determined based on actual requirements of the EMC circuit 60110 or experiences, which may not be limited in the present disclosure. A second performance may include that the elements in the EMC circuit 60110 have electromagnetic susceptibility (EMS) to prevent from being affected by the EMI.

In some embodiments, the EMC circuit 60110 may be configured to protect elements or components of the power supply assembly 60100 to work normally in an electromagnetic environment. That is, the EMC circuit 60110 may ensure that the EMI and signals coexist in the electromagnetic environment. As illustrated in FIG. 60, an input of the EMC circuit 60110 may be an input voltage. In some embodiments, the input voltage may be supplied by mains electricity. For example, the input voltage may be an alternating voltage of 220V. In some embodiments, an output of the EMC circuit 60110 may be connected with an input of the heat dissipation mechanism 60160. In some embodiments, an output may be connected with the isolated power supply 60120 and the non-isolated power supply 60130 via the heat dissipation mechanism 60160.

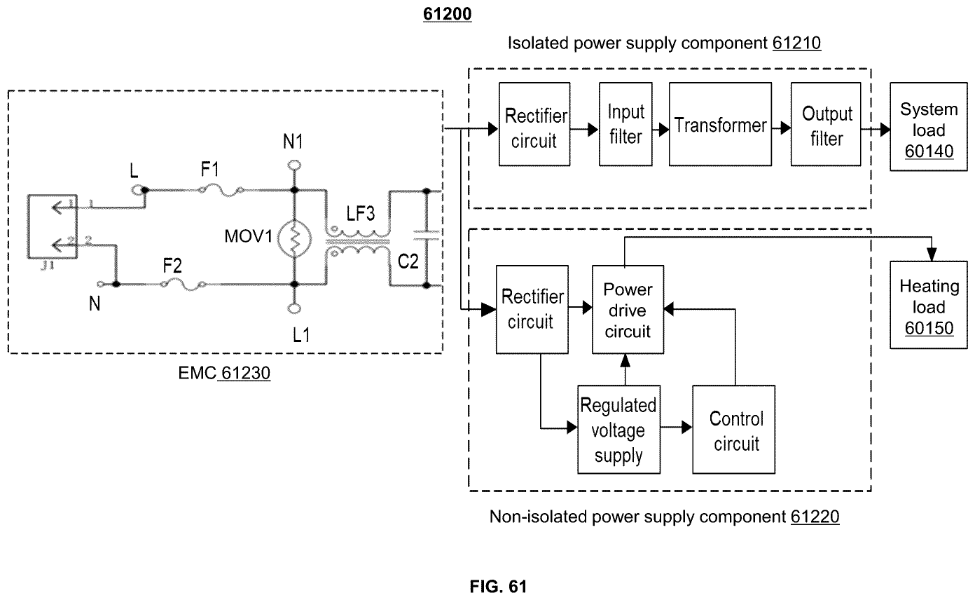

The isolated power supply component 60120 may be configured to supply power to a system load 60140 of an apparatus for ventilatory treatment (also referred to as a ventilatory treatment apparatus (e.g., the ventilatory treatment apparatus 200)). The isolated power supply component 60120 may refer to a power supply component that an input end and an output end thereof are isolated. When the isolated power supply component 60120 is working, a relatively high alternating current (AC) voltage inputted to an input circuit of the isolated power supply component 60120 may be transferred to a relatively low AC voltage via a transformer integrated in an output circuit of the isolated power supply component 60120, which may isolate the output circuit from the input circuit. Then, the relatively low AC voltage may be rectified to a relatively low direct current (DC) voltage, and the relatively low DC voltage may be outputted to supply power to a certain load. In some embodiments, the transformer may be a coupling of a plurality of inductance coils, thus the input circuit of the isolated power supply component 60120 may not be connected with an output circuit of the isolated power supply component 60120 directly, but the input circuit may be in electrical communication with the output circuit with the plurality of inductance coils. In this way, the high AC voltage inputted to the input circuit may be rectified to the low DC voltage in the output circuit, and different circuits in the isolated power supply component 60120 may not be affected by each other. An operator (e.g., a user of the ventilatory treatment apparatus) may not need to operate the isolated power supply component 60120 in a high voltage (e.g., mains electricity with 220V), and thus, the operation safety may be improved. However, because of the isolated connection of the input circuit and the output circuit, the transmission efficiency of electric signals or electric energy may be relatively low, and a volume of the isolated power supply component 60120 including the isolated input circuit and the isolated output circuit may be relatively large, thus the cost of producing the isolated power supply component 60120 may be increased. An exemplary isolated power supply component may be shown in FIG. 61.

The non-isolated power supply 60130 may be configured to supply power to a heating load 60150 of the apparatus. The non-isolated power supply component 60130 may refer to a power supply component that an input end and an output end thereof are connected with each other. For example, an input circuit of the non-isolated power supply component 60130 may be connected with an output circuit of the non-isolated power supply component 60130 directly. Though a direct connection of the input circuit and the output circuit may cause a high-risk operation for the operator, but the transmission efficiency of electric signals and electric energy may be improved because of the direct connection, and the difficulty of designing the circuit may be reduced, and the cost of producing the non-isolated power supply component 60130 may also be decreased. An exemplary non-isolated power supply component may be shown in FIG. 61.

The system load 60140 may refer to components or devices included in the ventilatory treatment apparatus. The components or devices may convert the power (e.g., the electric energy) supplied by the isolated power supply component 60120 to the mechanical energy. For example, the system load 60140 may include the gas mixing assembly 730, the gas pressurization unit 720, or the like.

The heating load 60150 may refer to other components or devices included in the ventilatory treatment apparatus. The other components or devices may convert the power (e.g., the electric energy) supplied by the non-isolated power supply component 60130 to the heat energy to heat a liquid. For example, the heating load 60150 may include the heater assembly 3740, or the like.

In some embodiments, the heat dissipation mechanism 60160 may be configured to dissipate heat generated in the EMC circuit 60110, the isolated power supply component 60120, and/or the non-isolated power supply component 60130. For example, a great amount of heat may cause a damage to one or more components of the EMC circuit 60110. The heat dissipation mechanism 60160 may dissipate the heat, thus the service life of the EMC circuit 60110 may be increased, and the operation safety may also be improved. In some embodiments, the heat dissipation mechanism 60160 may include an air-cooling heat dissipation mechanism, a water-cooling dissipation mechanism, or the like, which may not be limited in the present disclosure.

It should be noted that the descriptions of the power supply assembly 60100 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 61 illustrates an exemplary power supply assembly 61200 according to some embodiments of the present disclosure.

In some embodiments, in order to supply power to the system load 60140 of the ventilatory treatment apparatus, an isolated power supply component 61210 may be configured to convert an input voltage to a first target voltage required by the system load 60140. The first target voltage may refer to a work voltage of the apparatus. That is, the apparatus may work normally with the first target voltage.

As illustrated in FIG. 61, the isolated power supply component 61210 may include a rectifier circuit, an input filter, a transformer, and an output filter. In some embodiments, an input of the isolated power supply component 61210 may be mains electricity. The mains electricity may be alternating current (e.g., a voltage of 220V), which may not be suitable for using in the ventilatory treatment apparatus. Thus, the rectifier circuit of the isolated power supply component 61210 may be configured to rectify alternating current (AC) to direct current (DC). In some embodiments, the rectifier circuit of the isolated power supply component 61210 may include a diode, such as a solid-state diode, a vacuum tube diode, or the like.

In some embodiments, the input filter of the isolated power supply component 61210 may be provided between the rectifier circuit of the isolated power supply component 61210 and the transformer, and configured to filter interferences (e.g., conduction interferences or electromagnetic interferences) generated by the rectifier circuit of the isolated power supply component 61210. In some embodiments, the input filter may include an active filter, a passive filter, a ceramic filter, a crystal filter, a mechanical filter, or the like, or any combination thereof, which may not be limited in the present disclosure.

The transformer may be configured to facilitate voltage transformation (e.g., covert an input voltage inputted from the input filter to a first voltage of the apparatus). In some embodiments, the transformer may include a single-phase transformer, a three-phase transformer, or the like, which may not be limited in the present disclosure.

In some embodiments, an output end of the transformer may be connected with the output filter, thus the first voltage output from the transformer may be filtered in the output filter. Then the filtered first voltage may be the first target voltage, and may be supplied to the system load 60140 for working. In some embodiments, the output filter may include an active filter, a passive filter, a ceramic filter, a crystal filter, a mechanical filter, or the like, or any combination thereof, which may not be limited in the present disclosure.

In some embodiments, in order to supply power to the heating load 60150 of the apparatus, a non-isolated power supply component 61220 may be configured to convert an input voltage to a second target voltage required by the heating load 60150. The second target voltage may refer to a work voltage of the heating load 60150 of the apparatus, then the heating load 60150 may heat a liquid normally with the second target voltage.

As illustrated in FIG. 61, the non-isolated power supply component 61220 may include a rectifier circuit, a power drive circuit, a control circuit, and a regulated voltage supply. As a similar way with the isolated power supply component 61210, an input of the non-isolated power supply component 61220 may also be mains electricity. Thus, the rectifier circuit of the non-isolated power supply component 61220 may be configured to rectify AC provided from the mains electricity to direct current (DC). In some embodiments, the rectifier circuit of the non-isolated power supply component 61220 may include a diode, such as a solid-state diode, a vacuum tube diode, or the like.

In some embodiments, the heating load 60150 of the ventilatory treatment apparatus may be independent from other components or device of the apparatus. Thus, the heating load 60150 may need to be turned on or off independently. In some embodiments, the control circuit may be configured to control an on/off state of the power drive circuit, and the power drive circuit may be configured to drive the heating load 60150. For example, when the control circuit controls the power drive circuit being on an on state, the power drive circuit on the on state may allow the power being supplied to the heating load 60150, thus driving the heating load 60150 to work. Then the heating load 60150 (e.g., the heater assembly 3740) may start to heat a liquid. As another example, the control circuit may control the power drive circuit converting from the on state to an off state, then the power drive circuit may not allow the power being supplied to the heating load 60150, thus the heating load 60150 may stop working. In some embodiments, the power drive circuit may drive the heating load 60150 by supplying an electric signal with a relatively high power to the heating load 60150. In some embodiments, the electric signal may refer to an electric signal with enough power to excite the heating load 60150, which may not be limited in the present disclosure.

Since the control circuit may control an on/off state of the power drive circuit independently, an independent power supply may be needed to provide power for the control circuit and the power drive circuit. In some embodiments, the regulated voltage supply may be configured to supply power to the control circuit and the power drive circuit.

As illustrated in FIG. 61, the isolated power supply component 61210 and the non-isolated power supply component 61220 may share at least a portion of an EMC circuit 61230. In some embodiments, the EMC circuit 61230 may include a varistor (e.g., the MOV1 shown in FIG. 61) and a safety capacitor. In some embodiments, the varistor MOV1 may be disposed between a zero line (or null line) N1 and a fire line (or live line) L1, and may be in parallel connection with a magnet ring (e.g., LF3 shown in FIG. 61) of the EMC circuit 61230.

In some embodiments, the safety capacitor may include an X-capacitor (e.g., C2 shown in FIG. 61). The X-capacitor C2 may be disposed between the zero line N1 and the fire line L1, and may be in parallel connection with the magnet ring LF3 of the EMC circuit 61230. In some embodiments, the X-capacitor C2 may be configured to avoid electromagnetic interference (EMI) generated by other components in the EMC circuit 61230, thus to improve a performance of anti-interference capability of the EMC circuit 61230.

In some embodiments, the isolated power supply component 61210 may be disposed between two ends of the X-capacitor C2, and the non-isolated power supply component 61220 may be disposed between the zero line N1 and the fire line L1.

In some embodiments, the at least a portion of the EMC circuit 61230 may include the varistor MOV1 and one or more fuses. As shown in FIG. 61, the one more fuses may include a fuse F1 and/or a fuse F2. In some embodiments, the one or more fuses may be configured to protect the whole circuit. For example, if the EMC circuit has a short circuit, an electric current in the EMC circuit may be great, and the one or more fuses may blow, thus the EMC circuit may be broken, thus avoiding an overload caused by the great electric current. The service life of the components of the EMC circuit may be improved, and the operation safety may also be improved.

It should be noted that the descriptions of the isolated power supply components 61210, the non-isolated power supply components 61220 and the EMC circuit 61230 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

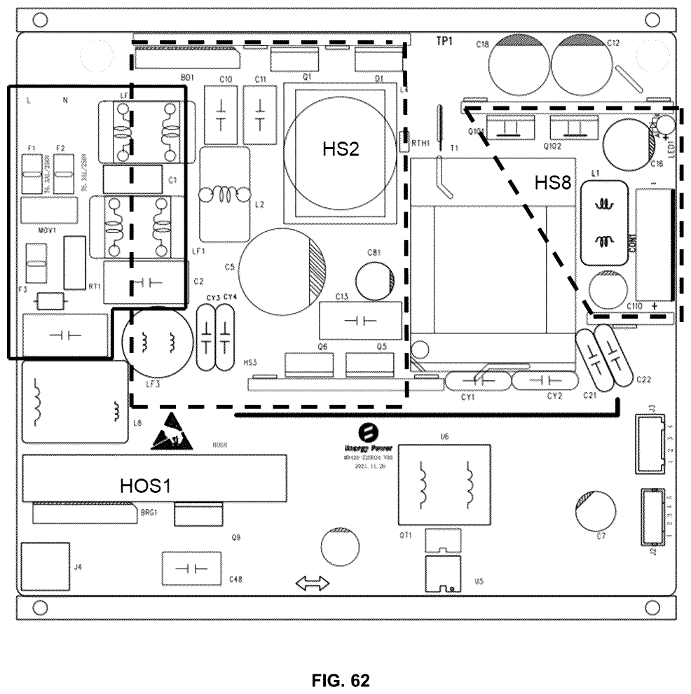

FIG. 62 illustrates an exemplary circuit of a power supply assembly according to some embodiments of the present disclosure.

As described above, an isolated power supply component and a non-isolated power supply component may share at least a portion of a heat dissipation mechanism, and the heat dissipation mechanism may be configured to dissipate heat generated in an EMC circuit, the isolated power supply component, and/or the non-isolated power supply component.

In some embodiments, the heat dissipation mechanism may include a first heat dissipation structure (e.g., HOS1 shown in FIG. 62), a second heat dissipation structure (e.g., HS2 (which is defined by dotted lines illustrated in FIG. 62)), a third heat dissipation structure (e.g., HS8 (which is defined by dotted lines illustrated in FIG. 62)). In some embodiment, the first heat dissipation structure HOS1 may be configured to dissipate heat of the EMC circuit (which is defined by solid lines illustrated in FIG. 62), the isolated power supply component, and the non-isolated power supply component. The second heat dissipation structure HS2 may be configured to dissipate heat of the EMC circuit and the isolated power supply component. The third heat dissipation structure may be configured to dissipate heat of the non-isolated power supply component. In some embodiments, the EMC circuit may be provided near a high voltage circuit. The high voltage circuit may include a circuit of the non-isolated power supply component since the input voltage may be supplied to the non-isolated power supply component directly.

In some embodiments, the first heat dissipation structure HOS1 may include an aluminum profile ultrasonic heat sink. In some embodiments, the second heat dissipation structure and/or the third heat dissipation structure may include a stamped aluminum heat sink.

It should be noted that the descriptions of the heat dissipation mechanism are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

The possible beneficial effects of the power supply assembly disclosed in the present disclosure may include, but be not limited to: (1) the power supply assembly may both include an isolated power supply component and an non-isolated power supply component, requirements of power supply of different components or devices of the ventilatory treatment apparatus may be satisfied, and the apparatus may be suitable for more application scenarios with different input voltages, thus the work efficiency of the apparatus may be improved; (2) the isolated power supply component and the non-isolated power supply component may be integrated in a single circuit board and share at least a portion of an EMC circuit and at least a portion of a heat dissipation mechanism, a space utilization of a circuit board of the power supply assembly may be improved, a size of the circuit board of the power supply assembly may be reduced, and thus the installation of the power supply assembly may be more convenient, and a production cost of the circuit board may be decreased; (3) the EMC circuit may be provided near a high voltage circuit (e.g., a circuit of the non-isolated power supply component) on the circuit board of the power supply assembly, thus a rule scope of a circuit of the non-isolated power supply component may be narrowed, and the space utilization may further be improved.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer-readable media having computer readable program code embodied thereon.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. An apparatus for ventilatory treatment, comprising:
at least one gas entrance configured to introduce a first gas into the apparatus and introduce a second gas into the apparatus;
a gas mixing assembly configured to mix the first gas and the second gas to generate a mixed gas;
a gas export configured to discharge a flow of the mixed gas; and
a noise reduction assembly configured to reduce noise of the apparatus.

2. The apparatus of claim 1, wherein the first gas includes a target gas, the second gas includes the target gas, and a second concentration ratio of the target gas in the second gas exceeds a first concentration ratio of the target gas in the first gas.

3. The apparatus of claim 2, wherein the target gas includes oxygen, the first gas includes air, and the second gas includes high-concentration oxygen with higher concentration of oxygen than air.

4. The apparatus of claim 1, further comprising a humidification assembly configured to humidify the mixed gas discharged from the gas mixing assembly.

5. The apparatus of claim 1, further comprising a gas pressurization unit configured to:
generate a pressurized first gas by pressurizing the first gas before the gas mixing assembly mixes the first gas and the second gas; or
generate a pressurized mixed gas by pressurizing the mixed gas after the gas mixing assembly mixes the first gas and the second gas.

6. The apparatus of claim 5, wherein
the gas pressurization unit includes a gas inlet port and a gas outlet port,
the gas inlet port of the gas pressurization unit is in fluid communication with the at least one gas entrance via a tube to receive the first gas from the at least one gas entrance,
the gas outlet port of the gas pressurization unit is in fluid communication with a first inlet port of the gas mixing assembly to introduce the pressurized first gas into the gas mixing assembly through the first inlet port of the gas mixing assembly.

7. The apparatus of claim 5, further comprising a gas importing mechanism configured to import the second gas, from at least one of a first gas source or a second gas source, via a gas outlet port of the gas importing mechanism and a second inlet port of the gas mixing assembly, into the gas mixing assembly.

8. The apparatus of claim 7, wherein the gas mixing assembly receives the pressurized first gas from a gas outlet port of the gas pressurization unit, and receives the second gas from the gas outlet port of the gas importing mechanism.

9. The apparatus of claim 7, wherein the gas mixing assembly receives the first gas from the at least one gas entrance, and receives the second gas from the gas outlet port of the gas importing mechanism, before the gas pressurization unit pressurizes the mixed gas.

10. The apparatus of claim 7, wherein the gas importing mechanism includes at least one of a first sub-mechanism or a second sub-mechanism, the first sub-mechanism being configured to import the second gas from the first gas source automatically, the second sub-mechanism being configured to import the second gas from the second gas source manually.

11. The apparatus of claim 10, wherein the first sub-mechanism includes:
a pressure relief valve configured to lower a pressure of the second gas introduced from the first gas source;
a proportional valve configured to control a flow of the second gas introduced from the first gas source.

12. The apparatus of claim 11, wherein the first sub-mechanism further includes:
a pressure sensor configured to detect a pressure of the second gas introduced from the first gas source before the second gas flows into the pressure relief valve;
a flow sensor configured to detect the flow of the second gas flowing into the gas mixing assembly.

13. The apparatus of claim 12, wherein the second sub-mechanism includes:
a one-way valve configured to control the second gas introduced from the second gas source to flow into the gas mixing assembly.

14. The apparatus of claim 13, wherein the second sub-mechanism share the flow sensor with the first sub-mechanism.

15. The apparatus of claim 13, wherein
the gas importing mechanism is further configured to switch between an automatic gas importing manner and a manual gas importing manner,
the automatic gas importing manner is configured to import the second gas from the first gas source and functioned by the first sub-mechanism,
the manual gas importing manner is configured to import the second gas from the second gas source and functioned by the second sub-mechanism.

16. The apparatus of claim 15, wherein
the switching between the automatic gas importing manner and the manual gas importing manner is performed by controlling an on/off state of the proportional valve,
when the proportional valve is on, the gas importing mechanism is switched to the automatic gas importing manner,
when the proportional valve is off, the gas importing mechanism is switched to the manual gas importing manner.

17. The apparatus of claim 7, wherein
the at least one gas entrance includes a first-second gas entrance and a second-second gas entrance,
the first-second gas entrance is configured to introduce the second gas from the first gas source into the apparatus,
the second-second gas entrance is configured to introduce the second gas from the second gas source into the apparatus.

18. The apparatus of claim 17, wherein the first-second gas entrance and the second-second gas entrance are disposed on or in fluid communication with the gas importing mechanism.

19. The apparatus of claim 1, further comprising a gas intake connector assembly configured to introduce the second gas from at least one gas source, via the at least one gas entrance, into the apparatus.

20. The apparatus of claim 19, wherein
the gas intake connector assembly is disposed at the at least one gas entrance,
the gas intake connector assembly is connected to the at least one gas source directly or via a tube.

21-113. (canceled)
